(19) United States
(12) Patent Application Publication
(10) Pub. No.: US 2024/0392117 A1
(43) Pub. Date: Nov. 28, 2024

(54) METHODS OF MAKING BARRIERS INCLUDING NANOPORES AND CROSSLINKED AMPHIPHILIC MOLECULES, AND BARRIERS FORMED USING SAME

(71) Applicant: Illumina, Inc., San Diego, CA (US)

(72) Inventors: Antonio Conde-Gonzalez, Cambridge (GB); Davide Garoldini, Cambridge (GB); Yuliia Vyborna, Sawston (GB); Charlotte Vacogne, Cambridge (GB); Istvan Kocsis, Cambridge (GB); Oliver Uttley, Haverhill (GB); Miguel Angel Aleman Garcia, Cambridge (GB); Alexandre Richez, Cambridge (GB)

(73) Assignee: Illumina, Inc., San Diego, CA (US)

(21) Appl. No.: 18/653,173

(22) Filed: May 2, 2024

Related U.S. Application Data
(60) Provisional application No. 63/504,401, filed on May 25, 2023.

(51) Int. Cl. C08L 23/06; C08L 23/26

(52) U.S. Cl. CPC C08L 23/06; C08L 23/26

(57) ABSTRACT

Methods of making barriers including nanopores and crosslinked amphiphilic molecules, and barriers made using the same, are provided herein. In some examples, a method of forming a barrier between first and second fluids includes forming at least one layer comprising a plurality of amphiphilic molecules, wherein the amphiphilic molecules comprise reactive moieties. The method may include using first crosslinking reactions of the reactive moieties to only partially crosslink amphiphilic molecules of the plurality to one another. The method may include, after using the first crosslinking reactions, inserting the nanopore into the at least one layer. The method may include, after inserting the nanopore, using second crosslinking reactions of the reactive moieties to further crosslink amphiphilic molecules of the plurality to one another.

### CROSS-REFERENCE TO RELATED APPLICATIONS

This application claims the benefit of U.S. Provisional Patent Application No. 63/504,401, filed on May 25, 2023 and entitled “Methods of Making Barriers Including Nanopores and Cross-Linked Amphiphilic Molecules, and Barriers Formed Using Same,” the entire contents of which are incorporated by reference herein.

### FIELD

This application relates to barriers that include amphiphilic molecules.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in an α-hemolysin nanopore. Or, for example, KF-DNA complexes have been distinguished on the basis of their properties when captured in an electric field atop an α-hemolysin nanopore. In still another example, polynucleotide sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a polynucleotide that is being synthesized. The charge blockade label is detected by the nanopore to determine the presence and identity of the incorporated nucleotide and thereby determine the sequence of a template polynucleotide. In still other examples, constructs include a transmembrane protein nanopore subunit and a nucleic acid handling enzyme.

However, such previously known devices, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved devices, systems, and methods for sequencing polynucleotides, which may include using membranes having nanopores disposed therein.

### SUMMARY

Methods of making barriers including nanopores and crosslinked amphiphilic molecules, and barriers formed using the same, are provided herein.

Some examples herein provide a method of forming a barrier between first and second fluids. The method may include forming at least one layer including a plurality of amphiphilic molecules, wherein the amphiphilic molecules include reactive moieties. The method may include using first crosslinking reactions of the reactive moieties to only partially crosslink amphiphilic molecules of the plurality to one another. The method may include, after using the first crosslinking reactions, inserting the nanopore into the at least one layer. The method may include, after inserting the nanopore, using second crosslinking reactions of the reactive moieties to further crosslink amphiphilic molecules of the plurality to one another.

In some examples, forming the at least one layer includes forming a first layer including a first plurality of the amphiphilic molecules, and forming a second layer including a second plurality of the amphiphilic molecules.

In some examples, the crosslinking reaction includes a polymerization reaction. In some examples, the reactive moieties are selected from the group consisting of an itaconic moiety, an N-carboxyanhydride moiety, a disulfyl pyridyl moiety, an N-hydroxy succinimide (NHS) ester, an acrylate moiety, a methacrylate moiety, an acrylamide moiety, a methacrylamide moiety, a styrenic moiety, a maleic moiety, a carboxylic acid moiety, a thiol moiety, an allyl moiety, a vinyl moiety, a propargyl moiety, and a maleimide moiety. In some examples, the polymerization reaction includes a ring-opening polymerization or a step-growth polymerization. In some examples, the method further includes initiating the polymerization reaction using an initiator. In some examples, the initiator includes a photoinitiator, a redox system, or photons. In some examples, the photoinitiator is selected from the group consisting of: 2,2-dimethoxy-2-phenylacetophenone, 2,2′-azobis(2-methylpropionamidine) dihydrochloride, 2-hydroxy-4′-(2-hydroxyethoxy)-2-methylpropiophenone, and lithium phenyl-2,4,6,-trimethylbenzoylphosphinate. In some examples, the redox system includes potassium persulfate or ammonium persulfate and N,N,N′,N′-tetramethylethylenediamine.

In some examples, the crosslinking reaction includes a coupling reaction. In some examples, the coupling reaction includes a thiol-ene click reaction, a thiol-yne click reaction, a strain-promoted alkyne-azide cycloaddition, an amide coupling, a thiol/aza-Michael reaction, a [2+2] cycloaddition, a thio-Michael click reaction, a condensation reaction, a [2+2] photocycloaddition, a protein-ligand interaction, host-guest chemistry, a disulfide formation, an imine formation, or an enamine formation. In some examples, the coupling reaction is initiated using an initiator. In some examples, the initiator includes a free-radical initiator, a redox system, a reducing agent, or photons. In some examples, the free-radical initiator includes 2-hydroxy-4′-(2-hydroxyethoxy)-2-methylpropiophenone, or 2,2′-azobis(2-methylpropionamidine) dihydrochloride. In some examples, the redox system includes potassium persulfate or ammonium persulfate and N,N,N′,N′-tetramethylethylenediamine. In some examples, the reducing agent includes tris(2-carboxyethyl) phosphine, dithiothreitol, sodium ascorbate, or a phosphine. In some examples, the reactive moieties include a propargyl moiety, an N-hydroxysuccinimide (NHS) ester, a disulfide pyridyl moiety, a lipoamido moiety, a propargyl moiety, an azide moiety, a DBCO moiety, a BCN moiety, an amine moiety, an activated carboxylic moiety, a dimethylmaleimide moiety, or a maleimide moiety.

In some examples, the reactive moieties are located at hydrophilic blocks of the amphiphilic molecules. In some examples, the reactive moieties are located at interfaces between hydrophilic blocks and hydrophobic blocks of the amphiphilic molecules. In some examples, the reactive moieties are located at hydrophilic blocks of the amphiphilic molecules.

In some examples, the amphiphilic molecules have an AB architecture. In some examples, the amphiphilic molecules have an ABA architecture. In some examples, the amphiphilic molecules have a BAB architecture.

In some examples, the amphiphilic molecules include poly(dimethyl siloxane) (PDMS). In some examples, the amphiphilic molecules include poly(isobutylene) (PIB). In some examples, the amphiphilic molecules include poly(ethylene oxide) (PEO).

In some examples, the at least one layer is formed using a hydrophobic liquid consisting essentially of hydrophobic, polymerizable monomers; and the hydrophobic liquid is disposed within the at least one layer. In some examples, the first cross-linking reactions at least partially crosslink the monomers with one another. In some examples, the first cross-linking reactions at least partially crosslink the monomers with amphiphilic molecules of the plurality. In some examples, the second cross-linking reactions at least partially crosslink the monomers with one another. In some examples, the second cross-linking reactions at least partially crosslink the monomers with amphiphilic molecules of the plurality. In some examples, at least some of the monomers include a single reactive moiety via which those monomers polymerize with other monomers or react with the reactive moieties of the amphiphilic molecules. In some examples, at least some of the monomers include two or more reactive moieties via which those monomers polymerize with other monomers or react with the reactive moieties of the amphiphilic molecules. In some examples, at least a portion of the monomers is intercalated between amphiphilic molecules of the at least one layer. In some examples, the at least one layer includes first and second layers, and at least a portion of the monomers is disposed between the first layer and the second layer. In some examples, the monomers include acrylate, and the polymer includes polyacrylate.

In some examples, the barrier is supported by a support having an aperture therethrough. In some examples, the polymer forms an overhanging annulus around the aperture. In some examples, the polymer substantially covers the aperture except where the nanopore is located. In some examples, the support includes reactive moieties, the method further including polymerizing the reactive moieties with the monomers.

In some examples, the nanopore includes a moiety that initiates the polymerization. In some examples, the nanopore includes a moiety that couples to a reactive moiety of an amphiphilic molecule. In some examples, the nanopore includes α-hemolysin. In some examples, the nanopore includes MspA.

Some examples herein provide a barrier between first and second fluids. The barrier may include at least one layer including a plurality of amphiphilic molecules and a polymer. At least some amphiphilic molecules of the plurality of amphiphilic molecules are crosslinked to one another, and at least some amphiphilic molecules of the plurality of amphiphilic molecules are crosslinked to the polymer.

In some examples, the at least one layer includes a first layer including a first plurality of amphiphilic molecules; and a second layer including a second plurality of amphiphilic molecules contacting the first plurality of amphiphilic molecules. In some examples, at least some amphiphilic molecules of the first layer are crosslinked to one another, and at least some amphiphilic molecules of the second layer are crosslinked to one another. In some examples, the amphiphilic molecules include at least one hydrophobic block coupled to at least one hydrophilic block at an interface. In some examples, at least some of the amphiphilic molecules are crosslinked to one another at the hydrophilic blocks. In some examples, at least some of the amphiphilic molecules are crosslinked to one another at the hydrophobic blocks. In some examples, at least some of the amphiphilic molecules are crosslinked to one another at the interface.

In some examples, the amphiphilic molecules include molecules of a diblock copolymer, molecules of the diblock copolymer including a hydrophobic block coupled to a hydrophilic block. In some examples, the amphiphilic molecules include molecules of a triblock copolymer. In some examples, each molecule of the triblock copolymer includes first and second hydrophobic blocks and a hydrophilic block coupled to and between the first and second hydrophobic blocks. In some examples, each molecule of the triblock copolymer includes first and second hydrophilic blocks and a hydrophobic block coupled to and between the first and second hydrophilic blocks.

In some examples, the amphiphilic molecules are crosslinked by a product of a polymerization reaction. In some examples, the product of the polymerization reaction includes a reacted itaconic moiety, a reacted N-carboxyanhydride moiety, a reacted disulfyl pyridyl moiety, a reacted N-hydroxy succinimide (NHS) ester, a reacted acrylate moiety, a reacted methacrylate moiety, a reacted acrylamide moiety, a reacted methacrylamide moiety, a reacted styrenic moiety, a reacted maleic moiety, a reacted carboxylic acid moiety, a reacted thiol moiety, a reacted allyl moiety, a reacted vinyl moiety, a reacted propargyl moiety, or a reacted maleimide moiety.

In some examples, the amphiphilic molecules are crosslinked by a product of a coupling reaction. In some examples, the coupling reaction includes a thiol-ene click reaction, a thiol-yne click reaction, a strain-promoted alkyne-azide cycloaddition, an amide coupling, a thiol/aza-Michael reaction, a [2+2] cycloaddition, a thio-Michael click reaction, a condensation reaction, a [2+2] photocycloaddition, a protein-ligand interaction, host-guest chemistry, a disulfide formation, an imine formation, or an enamine formation.

In some examples, the barrier further includes a nanopore within the barrier. In some examples, the nanopore includes a moiety that couples to a reactive moiety of an amphiphilic molecule. In some examples, the nanopore includes α-hemolysin or MspA.

In some examples, at least a portion of the polymer is intercalated between amphiphilic molecules of the at least one layer. In some examples, the at least one layer includes first and second layers, and at least a portion of the polymer is disposed between the first layer and the second layer.

In some examples, the polymer includes polyacrylate.

In some examples, the barrier is supported by a support having an aperture therethrough. In some examples, the polymer forms an overhanging annulus around the aperture. In some examples, the polymer substantially covers the aperture.

Some examples herein provide a barrier between first and second fluids. The barrier may include at least one layer including a plurality of amphiphilic molecules, wherein the amphiphilic molecules include reactive moieties to perform a crosslinking reaction with one another. In some examples, only a subset of the amphiphilic molecules are crosslinked with one another via a reaction product of the crosslinking reaction. A nanopore may be disposed within the barrier.

In some examples, the at least one layer includes a first layer including a first plurality of the amphiphilic molecules; and a second layer including a second plurality of the amphiphilic molecules contacting the first plurality of amphiphilic molecules.

In some examples, the reactive moieties are selected from the group consisting of an itaconic moiety, an N-carboxyanhydride moiety, a disulfyl pyridyl moiety, an N-hydroxy succinimide (NHS) ester, an acrylate moiety, a methacrylate moiety, an acrylamide moiety, a methacrylamide moiety, a styrenic moiety, a maleic moiety, a carboxylic acid moiety, a thiol moiety, an allyl moiety, a vinyl moiety, a propargyl moiety, and a maleimide moiety.

In some examples, the reactive moieties include a mixture of moieties that are reactive with one another via a thiol-ene click reaction, a thiol-yne click reaction, a strain-promoted alkyne-azide cycloaddition, an amide coupling, a thiol/aza-Michael reaction, a [2+2] cycloaddition, a thio-Michael click reaction, a condensation reaction, a [2+2] photocycloaddition, a protein-ligand interaction, host-guest chemistry, a disulfide formation, an imine formation, or an enamine formation.

In some examples, the amphiphilic molecules include at least one hydrophobic block coupled to at least one hydrophilic block at an interface. In some examples, the reactive moieties are located at the hydrophilic blocks of respective amphiphilic molecules. In some examples, the reactive moieties are located at the hydrophobic blocks of respective amphiphilic molecules. In some examples, the reactive moieties are located at the interfaces of respective amphiphilic molecules.

In some examples, the amphiphilic molecules include molecules of a diblock copolymer, molecules of the diblock copolymer including a hydrophobic block coupled to a hydrophilic block. In some examples, the amphiphilic molecules include molecules of a triblock copolymer. In some examples, each molecule of the triblock copolymer including first and second hydrophobic blocks and a hydrophilic block coupled to and between the first and second hydrophobic blocks. In some examples, each molecule of the triblock copolymer including first and second hydrophilic blocks and a hydrophobic block coupled to and between the first and second hydrophilic blocks.

In some examples, the barrier further includes a hydrophobic liquid consisting essentially of hydrophobic, polymerizable monomers, wherein the hydrophobic liquid is disposed within the at least one layer. In some examples, the monomers are partially crosslinked with one another to form the polymer. In some examples, the monomers are partially crosslinked with amphiphilic molecules of the plurality. In some examples, at least some of the monomers include a single reactive moiety via which those monomers can polymerize with other monomers or react with the reactive moieties of the amphiphilic molecules. In some examples, at least some of the monomers include two or more reactive moieties via which those monomers can polymerize with other monomers or react with the reactive moieties of the amphiphilic molecules. In some examples, at least a portion of the monomers is intercalated between amphiphilic molecules of the at least one layer. In some examples, the at least one layer includes first and second layers, and at least a portion of the monomers is disposed between the first layer and the second layer. In some examples, the monomers include acrylate. In some examples, the polymer includes polyacrylate.

In some examples, the barrier is supported by a support having an aperture therethrough. In some examples, the polymer forms an overhanging annulus around the aperture. In some examples, the polymer substantially covers the aperture except where the nanopore is located. In some examples, the support includes reactive moieties, the method further including polymerizing the reactive moieties with the monomers.

In some examples, the nanopore includes a moiety that initiates the polymerization. In some examples, the nanopore includes a moiety that couples to a reactive moiety of an amphiphilic molecule or to the polymer. In some examples, the nanopore includes α-hemolysin. In some examples, the nanopore includes MspA.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates a cross-sectional view of an example nanopore composition and device including a barrier.

FIGS. 2A-2B schematically illustrate plan and cross-sectional views of further details of the nanopore composition and device of FIG. 1.

FIGS. 3A-3F schematically illustrate example operations for forming a barrier including a nanopore and crosslinked amphiphilic molecules.

FIGS. 4A-4D schematically illustrate example operations for forming an alternative barrier including a nanopore and crosslinked amphiphilic molecules.

FIGS. 5A-5D schematically illustrate example operations for forming an alternative barrier including a nanopore and crosslinked amphiphilic molecules.

FIGS. 6A-6B schematically illustrate example operations for forming an alternative barrier including a nanopore and crosslinked amphiphilic molecules.

FIGS. 7A-7B schematically illustrate example operations for forming an alternative barrier including a nanopore and crosslinked amphiphilic molecules.

FIGS. 8A-8B schematically illustrate example operations for forming an alternative barrier including a nanopore and crosslinked amphiphilic molecules.

FIGS. 9A-9B schematically illustrate example operations for forming an alternative barrier including a nanopore and crosslinked amphiphilic molecules.

FIGS. 10A-10B schematically illustrate example operations for forming an alternative barrier including a nanopore and crosslinked amphiphilic molecules.

FIGS. 11A-11B schematically illustrate example operations for forming an alternative barrier including a nanopore and crosslinked amphiphilic molecules.

FIG. 11C schematically illustrates example diblock copolymer molecules that may be used in operations such as described with reference to FIG. 5A-5D, 6A-6B, or 11A-11B.

FIG. 12 schematically illustrates an example operation for forming an alternative barrier including a nanopore and crosslinked amphiphilic molecules.

FIGS. 13A-13B schematically illustrate example operations for forming an alternative barrier including a nanopore and crosslinked amphiphilic molecules.

FIG. 13C schematically illustrates example triblock copolymer molecules that may be used in operations such as described with reference to FIG. 4A-4D, 9A-9B, or 13A-13B.

FIG. 14 schematically illustrates an alternative manner in which the operations described with reference to FIG. 3C or 3F may be performed.

FIG. 15 schematically illustrates an alternative manner in which the operations described with reference to FIG. 3C or 3F may be performed.

FIG. 16 schematically illustrates an example manner in which a barrier may be covalently coupled to a nanopore during operations such as described with reference to FIGS. 3A-3F.

FIG. 17 schematically illustrates an example manner in which a barrier may be covalently coupled to a barrier support during operations such as described with reference to FIGS. 3A-3F.

FIGS. 18A-18C schematically illustrate further details of barriers using block copolymers which may be included in the nanopore composition and device of FIG. 1 and used in respective operations described with reference to FIGS. 3A-17.

FIG. 19 illustrates an example flow of operations in a method for forming a barrier including cross-linked amphiphilic molecules.

FIG. 20A schematically illustrates an example reaction to form barriers that are crosslinked as described with reference to FIG. 4A-4D, 5A-5D, 6A-6B, 8A-8B, or 9A-9B.

FIG. 20B schematically illustrates an example reaction to form barriers that are crosslinked as described with reference to FIG. 2A-2B, 3A-3F, 7A-7B, or 10A-10B.

FIG. 21A schematically illustrates an example reaction to form barriers that are crosslinked as described with reference to FIGS. 11A-11B.

FIG. 21B schematically illustrates an example reaction to form barriers that are crosslinked as described with reference to FIG. 4A-4D, 5A-5D, 6A-6B, 8A-8B, or 9A-9B.

FIG. 22A schematically illustrates an example reaction to form barriers that are crosslinked as described with reference to FIG. 4A-4D, 5A-5D, 6A-6B, 8A-8B, or 9A-9B.

FIG. 22B schematically illustrates an example reaction to form barriers that are crosslinked as described with reference to FIG. 4A-4D, 5A-5D, 6A-6B, 8A-8B, or 9A-9B.

FIG. 23A schematically illustrates an example reaction to form barriers that are crosslinked as described with reference to FIGS. 11A-11B.

FIG. 23B schematically illustrates an example reaction to form barriers that are crosslinked as described with reference to FIGS. 11A-11B.

FIGS. 24-27 depict example chemical reactions between different reactive moieties in different locations of the block copolymer.

FIG. 28 schematically illustrates a cross-sectional view of an example use of the composition and device of FIG. 1.

FIG. 29 schematically illustrates a cross-sectional view of another example use of the composition and device of FIG. 1.

FIG. 30 schematically illustrates a cross-sectional view of another example use of the composition and device of FIG. 1.

FIG. 31 schematically illustrates a cross-sectional view of another example use of the composition and device of FIG. 1.

FIG. 32 schematically illustrates a cross-sectional view of another example use of the composition and device of FIG. 1.

FIGS. 33A-33B are plots illustrating the measured stability of barriers formed in the manner described with reference to FIGS. 3A-3C.

FIG. 34 is a plot illustrating the normalized number of nanopores remaining in the barrier during operations described with reference to FIGS. 3A-3F.

FIG. 35 is a plot illustrating normalized number of nanopores remaining in a barrier formed in the manner described with reference to FIGS. 3A-3F under different applied voltages.

FIG. 36 is a plot illustrating normalized number of nanopores remaining in a barrier formed in the manner described with reference to FIGS. 3A-3F using different processing parameters, under different applied voltages.

FIG. 37 illustrates a stiffness profile obtained using atomic force microscopy (AFM) imaging of suspended barriers after different operations described with reference to FIGS. 3A-3F.

### DETAILED DESCRIPTION

Methods of forming barriers including nanopores and cross-linked amphiphilic molecules, and barriers formed using the same, are provided herein.

For example, nanopore sequencing may utilize a nanopore that is inserted into a barrier, such as a polymeric membrane, and that includes an aperture through which ions and/or other molecules may flow from one side of the barrier to the other. Circuitry may be used to detect a sequence of nucleotides. For example, during sequencing-by-synthesis (SBS), on a first side of the barrier, a polymerase adds the nucleotides to a growing polynucleotide in an order that is based on the sequence of a template polynucleotide to which the growing polynucleotide is hybridized. The sensitivity of the circuitry may be improved by using fluids with different compositions on respective sides of the barrier, for example to provide suitable ion transport for detection on one side of the barrier, while suitably promoting activity of the polymerase on the other side of the barrier. Accordingly, barrier stability is beneficial.

As provided herein, a barrier including a nanopore and amphiphilic molecules may be stabilized by partially cross-linking the amphiphilic molecules, then inserting the nanopore, then increasing cross-linking of the amphiphilic molecules. Illustratively, the amphiphilic molecules may be or include polymer chains that include functional groups at their respective hydrophilic (A) ends, at their respective hydrophobic (B) ends, or at the hydrophilic-hydrophobic (A-B) interface, or at combinations of such locations (e.g., at the hydrophilic ends and/or at the hydrophobic ends and/or at the hydrophilic-hydrophobic interface). The functional groups may be reacted in such a manner as to partially cross-link the amphiphilic molecules before nanopore insertion, and then further cross-link the amphiphilic molecules after nanopore insertion. As explained herein, such an order of operations reduces the likelihood of the barrier ejecting the nanopore, while enhancing barrier stability. Accordingly, the barrier may be expected to be sufficiently strong and stable for prolonged use under forces such as may be applied during use of a device including such a barrier, illustratively genomic sequencing. Additionally, as described in greater detail below, a wide variety of different cross-linking chemistries suitably may be used, such as polymerization reactions or covalent coupling reactions.

First, some terms used herein will be briefly explained. Then, some example methods for forming barriers including nanopores and cross-linked amphiphilic molecules, and barriers formed using such methods, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic (polymer) materials or components such as polyethylene, polystyrene, poly(vinyl chloride), polypropylene, nylons, polyesters, polycarbonates, poly(methyl methacrylate), SU-8 type material, polyetherimide, and KMPR® resists (which is a high-contrast, epoxy based photoresist which can be developed in an aqueous alkaline developer and is commercially available from Kayaku Advanced Materials, Westborough, MA). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

As used herein, the term “electrode” is intended to mean a solid structure that conducts electricity. Electrodes may include any suitable electrically conductive material, such as gold, palladium, silver, or platinum, or combinations thereof. In some examples, an electrode may be disposed on a substrate. In some examples, an electrode may define a substrate.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules such as amino acids or nucleotides. The nanopore can be disposed within a barrier (such as membrane), or can be provided through a substrate. In some embodiments, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. nanopores include biological nanopores, solid-state nanopores, or biological and solid-state hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an α-helix bundle nanopore and a β-barrel nanopore as well as all others well known in the art. Example polypeptide nanopores include aerolysin, α-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, CsgG, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105:20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Norcadia farcinica, and lysenin. For further details regarding lysenin, see PCT Publication No. WO 2013/153359, the entire contents of which are incorporated by reference herein.

A “polynucleotide nanopore” is intended to mean a nanopore that is made from one or more nucleic acid polymers. A polynucleotide nanopore can include, for example, a polynucleotide origami.

A “solid-state nanopore” is intended to mean a nanopore that is made from one or more materials that are not of biological origin. A solid-state nanopore can be made of inorganic or organic materials. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state barrier, e.g., a barrier including any such material(s).

A “biological and solid-state hybrid nanopore” is intended to mean a hybrid nanopore that is made from materials of both biological and non-biological origins. Materials of biological origin are defined above and include, for example, polypeptides and polynucleotides. A biological and solid-state hybrid nanopore includes, for example, a polypeptide-solid-state hybrid nanopore and a polynucleotide-solid-state nanopore.

As used herein, a “barrier” is intended to mean a structure that normally inhibits passage of molecules from one side of the barrier to the other side of the barrier. The molecules for which passage is inhibited can include, for example, ions and water-soluble molecules such as nucleotides or amino acids. However, if a nanopore is disposed within a barrier, then the aperture of the nanopore may permit passage of molecules from one side of the barrier to the other side of the barrier. As one specific example, if a nanopore is disposed within a barrier, the aperture of the nanopore may permit passage of molecules from one side of the barrier to the other side of the barrier. Barriers include membranes of biological origin, such as lipid bilayers, and non-biological barriers such as solid-state barriers or substrates.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, “synthetic” refers to a barrier material that is not of biological origin (e.g., polymeric materials, synthetic phospholipids, solid-state barriers, or combinations thereof).

As used herein, a “polymeric membrane,” “polymeric barrier,” “polymer barrier,” or a “polymer membrane” refers to a synthetic barrier that primarily is composed of a polymer that is not of biological origin. In some examples, a polymeric membrane consists essentially of a polymer that is not of biological origin. A block copolymer is an example of a polymer that is not of biological origin and that may be included in the present barriers. A hydrophobic polymer with ionic end groups is another example of a polymer that is not of biological origin and that may be included in the present barriers. Because the present barriers relate to polymers that are not of biological origin, the terms “polymeric membrane,” “polymer membrane,” “polymeric barrier,” “polymer barrier,” “membrane,” and “barrier” may be used interchangeably herein when referring to the present barriers, even though the terms “barrier” and “membrane” generally may encompass other types of materials as well.

As used herein, the term “block copolymer” is intended to refer to a polymer having at least a first portion or “block” that includes a first type of monomer, and at least a second portion or “block” that is coupled directly or indirectly to the first portion and includes a second, different type of monomer. The first portion may include a polymer of the first type of monomer, or the second portion may include a polymer of the second type of monomer, or the first portion may include a polymer of the first type of monomer and the second portion may include a polymer of the second type of monomer. In some embodiments, the first portion may include an end group with a hydrophilicity that is different than that of the first type of monomer, or the second portion may include an end group with a hydrophilicity that is different than that of the second type of monomer, or the first portion may include an end group with a hydrophilicity that is different than that of the first type of monomer and the second portion may include an end group with a hydrophilicity that is different than that of the second type of monomer. The end groups of any hydrophilic blocks may be located at an outer surface of a barrier formed using such hydrophilic blocks. Depending on the particular configuration, the end groups of any hydrophobic blocks may be located at an inner surface of the barrier or at an outer surface of a barrier formed using such hydrophobic blocks.

Block copolymers include, but are not limited to, diblock copolymers and triblock copolymers.

A “diblock copolymer” is intended to refer to a block copolymer that includes, or consists essentially of, first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block.

A “triblock copolymer” is intended to refer to a block copolymer that includes, or consists essentially of, first, second, and third blocks coupled directly or indirectly to one another. The first and third blocks may include, or may consist essentially of, the same type of monomer as one another, and the second block may include a different type of monomer. In some examples, the first block may be hydrophobic, the second block may be hydrophilic, and the third block may be hydrophobic and includes the same type of monomer as the first block, in which case the triblock copolymer may be referred to as a “BAB” copolymer where “A” refers to the hydrophilic block and “B” refers to the hydrophobic blocks. In other examples, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block.

The particular arrangement of molecules of polymer chains (e.g., block copolymers) within a polymeric barrier may depend, among other things, on the respective block lengths, the type(s) of monomers used in the different blocks, the relative hydrophilicities and hydrophobicities of the blocks, the composition of the fluid(s) within which the barrier is formed, and/or the density of the polymeric chains within the barrier. During formation of the barrier, these and other factors generate forces between molecules of the polymeric chains which laterally position and reorient the molecules in such a manner as to substantially minimize the free energy of the barrier. The barrier may be considered to be substantially “stable” once the polymeric chains have completed these rearrangements, even though the molecules may retain some fluidity of movement within the barrier.

An “A-B interface” of a block copolymer (such as a diblock or triblock copolymer) refers to the interface at which the hydrophilic block is coupled to the hydrophobic block.

As used herein, the term “hydrophobic” is intended to mean tending to exclude water molecules. Hydrophobicity is a relative concept relating to the polarity difference of molecules relative to their environment. Non-polar (hydrophobic) molecules in a polar environment will tend to associate with one another in such a manner as to reduce contact with polar (hydrophilic) molecules to a minimum to lower the free energy of the system as a whole.

As used herein, the term “hydrophilic” is intended to mean tending to bond to water molecules. Polar (hydrophilic) molecules in a polar environment will tend to associate with one another in such a manner as to reduce contact with non-polar (hydrophobic) molecules to a minimum to lower the free energy of the system as a whole.

As used herein, the term “amphiphilic” is intended to mean having both hydrophilic and hydrophobic properties. For example, a block copolymer that includes a hydrophobic block and a hydrophilic block may be considered to be “amphiphilic.” Illustratively, AB copolymers, ABA copolymers, and BAB copolymers all may be considered to be amphiphilic. Additionally, molecules including a hydrophobic polymer coupled to ionic end groups may be considered to be amphiphilic.

As used herein, a “solution” is intended to refer to a homogeneous mixture including two or more substances. In such a mixture, a solute is a substance which is uniformly dissolved in another substance referred to as a solvent. A solution may include a single solute, or may include a plurality of solutes. Additionally, or alternatively, a solution may include a single solvent, or may include a plurality of solvents. An “aqueous solution” refers to a solution in which the solvent is, or includes, water.

A first liquid that forms a homogeneous mixture with a second liquid is referred to herein as being “miscible” or “soluble” with the second liquid.

As used herein, the term “electroporation” means the application of a voltage across a barrier such that a nanopore is inserted into the barrier.

As used herein, terms such as “cross-linked” and “cross-linking” refer to the forming of a bond between molecules. The bond may include a covalent bond or a non-covalent bond, such as an ionic bond, a hydrogen bond, or π-π stacking. The molecules which are cross-linked may include polymers, proteins, or polymers and proteins.

As used herein, the term “initiator” is intended to mean an entity that can initiate a polymerization reaction. Nonlimiting examples of initiators include moieties, molecules, and/or photons that can initiate a polymerization reaction.

As used herein, terms such as “covalently coupled” or “covalently bonded” refer to the forming of a chemical bond that is characterized by the sharing of pairs of electrons between atoms. For example, a covalently coupled molecule refers to a molecule that forms a chemical bond, as opposed to a non-covalent bond such as electrostatic interaction.

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” or “C₁₋₄alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means fluorine, chlorine, bromine, or iodine, with fluorine and chlorine being examples.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” or “C₁₋₄alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

Groups that include an alkenyl group include optionally substituted alkenyl, cycloalkenyl, and heterocycloalkenyl groups.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” or “C₂₋₄alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

Groups that include an alkynyl group include optionally substituted alkynyl, cycloalkynyl, and heterocycloalkynyl groups.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some examples, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “heterocycle” refers to a cyclic compound which includes atoms of carbon along with another atom (heteroatom), for example nitrogen, oxygen or sulfur. Heterocycles may be aromatic (heteroaryl) or aliphatic. An aliphatic heterocycle may be completely saturated or may contain one or more or two or more double bonds, for example the heterocycle may be a heterocycloalkyl. The heterocycle may include a single heterocyclic ring or multiple heterocyclic rings that are fused.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some examples, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” or “cycloalkene” means a carbocyclyl ring or ring system having at least one double bond, wherein no ring in the ring system is aromatic. An example is cyclohexenyl or cyclohexene. Another example is norbornene or norbornenyl.

As used herein, “heterocycloalkenyl” or “heterocycloalkene” means a carbocyclyl ring or ring system with at least one heteroatom in ring backbone, having at least one double bond, wherein no ring in the ring system is aromatic. In some examples, heterocycloalkenyl or heterocycloalkene ring or ring system is 3-membered, 4-membered, 5-membered, 6-membered, 7-membered, 8-membered, 9-membered, or 10-membered.

As used herein, “cycloalkynyl” or “cycloalkyne” means a carbocyclyl ring or ring system having at least one triple bond, wherein no ring in the ring system is aromatic. An example is cyclooctyne. Another example is bicyclononyne. Another example is dibenzocyclooctyne (DBCO).

As used herein, “heterocycloalkynyl” or “heterocycloalkyne” means a carbocyclyl ring or ring system with at least one heteroatom in ring backbone, having at least one triple bond, wherein no ring in the ring system is aromatic. In some examples, heterocycloalkynyl or heterocycloalkyne ring or ring system is 3-membered, 4-membered, 5-membered, 6-membered, 7-membered, 8-membered, 9-membered, or 10-membered.

As used herein, “heterocycloalkyl” means a non-aromatic cyclic ring or ring system containing at least one heteroatom in the ring backbone. Heterocycloalkyls may be joined together in a fused, bridged or spiro-connected fashion. Heterocycloalkyls may have any degree of saturation provided that at least one heterocyclic ring in the ring system is not aromatic. The heterocycloalkyl group may have 3 to 20 ring members (i.e., the number of atoms making up the ring backbone, including carbon atoms and heteroatoms), although the present definition also covers the occurrence of the term “heterocycloalkyl” where no numerical range is designated. The heterocycloalkyl group may also be a medium size heterocycloalkyl having 3 to 10 ring members. The heterocycloalkyl group could also be a heterocycloalkyl having 3 to 6 ring members. The heterocycloalkyl group may be designated as “3-6 membered heterocycloalkyl” or similar designations. In some six membered monocyclic heterocycloalkyls, the heteroatom(s) are selected from one up to three of O, N or S, and in some five membered monocyclic heterocycloalkyls, the heteroatom(s) are selected from one or two heteroatoms selected from O, N, or S. Examples of heterocycloalkyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆) alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆) alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy (C₁-C₆) alkyl (i.e., ether), aryloxy, sulfhydryl (mercapto), halo(C₁-C₆) alkyl (e.g., —CF₃), halo(C₁-C₆)alkoxy (e.g., —OCF₃), C₁-C₆ alkylthio, arylthio, amino, amino(C₁-C₆) alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

Where the compounds disclosed herein have at least one stereocenter, they may exist as individual enantiomers or diastereomers, or as mixtures of such isomers, including racemates. Separation of the individual isomers or selective synthesis of the individual isomers is accomplished by application of various methods which are well known to practitioners in the art. Where compounds disclosed herein are understood to exist in tautomeric forms, all tautomeric forms are included in the scope of the structures depicted. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

As used herein, the term “adduct” is intended to mean the product of a chemical reaction between two or more molecules, where the product contains all of the atoms of the molecules that were reacted.

As used herein, the term “linker” is intended to mean a molecule or molecules via which one element is attached to another element. For example, a linker may attach a first reactive moiety to a second reactive moiety. Linkers may be covalent, or may be non-covalent. Nonlimiting examples of covalent linkers include alkyl chains, polyethers, amides, esters, aryl groups, polyaryls, and the like. Nonlimiting examples of noncovalent linkers include host-guest complexation, cyclodextrin/norbornene, adamantane inclusion complexation with β-CD, DNA hybridization interactions, streptavidin/biotin, and the like.

As used herein, the term “barrier support” is intended to refer to a structure that can suspend a barrier. A barrier support may define an aperture, such that a first portion of the barrier is suspended across the aperture, and a second portion of the barrier is disposed on, and supported by, the barrier. The barrier support may include any suitable arrangement of elements to define an aperture and suspend the barrier across the aperture. In some examples, a barrier support may include a substrate having an aperture defined therethrough, across which aperture the barrier may be suspended. Additionally, or alternatively, the barrier support may include one or more first features (such as one or more lips or ledges of a well within a substrate) that are raised relative to one or more second features (such as a bottom surface of the well), wherein a height difference between (a) the one or more first features and (b) the one or more second features defines an aperture across which a barrier may be suspended. The aperture may have any suitable shape, such as a circle, an oval, a polygon, or an irregular shape. The barrier support may include any suitable material or combination of materials. For example, the barrier support may be of biological origin, or may be solid state. Some examples, the barrier support may include, or may consist essentially of, an organic material, e.g., a curable resin such as SU-8 or KMPR®; polytetrafluoroethylene (PTFE), poly(methyl methacrylate) (PMMA), parylene, or the like. Additionally, or alternatively, various examples, the barrier support may include, or may consist essentially of, an inorganic material, e.g., silicon nitride, silicon oxide, or molybdenum disulfide.

As used herein, the term “annulus” is intended to refer to a liquid that is adhered to a barrier support, located within a barrier, and extends partially into an aperture defined by the barrier support. As such, it will be understood that the annulus may follow the shape of the aperture of the barrier, e.g., may have the shape of a circle, an oval, a polygon, or an irregular shape.

As used herein, the term “monomer” is intended to mean a molecule that is bonded to one or more other molecules to form a polymer, or that is not yet bonded to one or more other molecules to form a polymer and is capable of being bonded to one or more other molecules to form a polymer. In examples in which the monomer is not yet bonded to one or more other molecules, the monomer can react with at least one other such molecule responsive to an initiator. The product of the reaction may be referred to as a “polymer” because it includes at least two such reacted monomers. A polymer also may include the products of reaction between different monomers. For example, a polymer may include multiple ones of a first type of molecule and may include one or more of a second type of molecule.

### Methods of Making Barriers Including Nanopores and Cross-Linked Amphiphilic Molecules, and Barriers Formed Using the Same

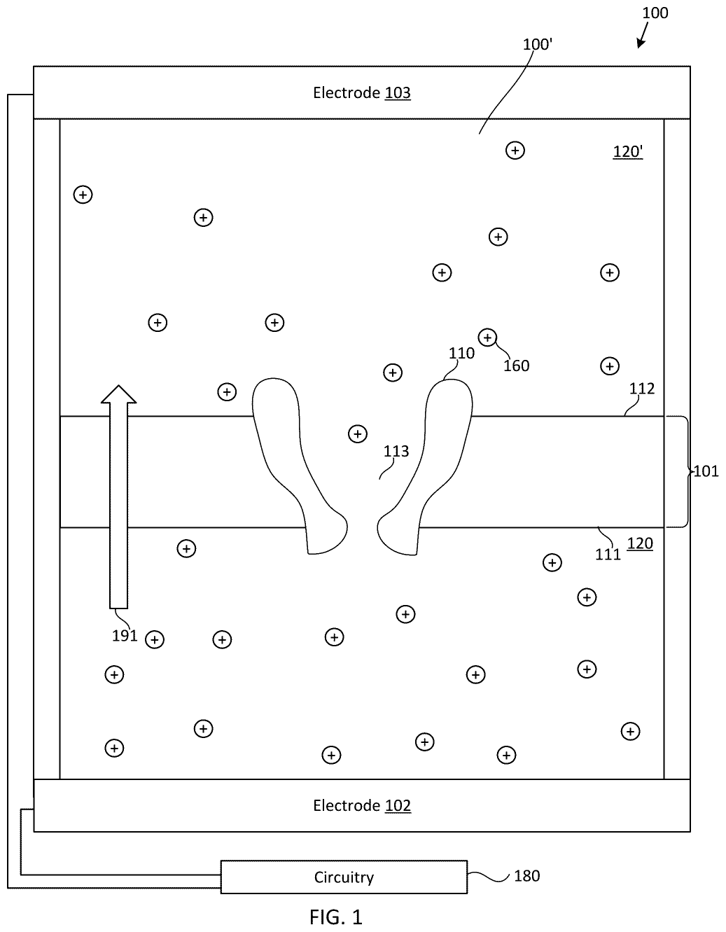

Methods of making barriers including nanopores and cross-linked amphiphilic molecules, and barriers formed using the same, now will be described with reference to FIGS. 1-27.

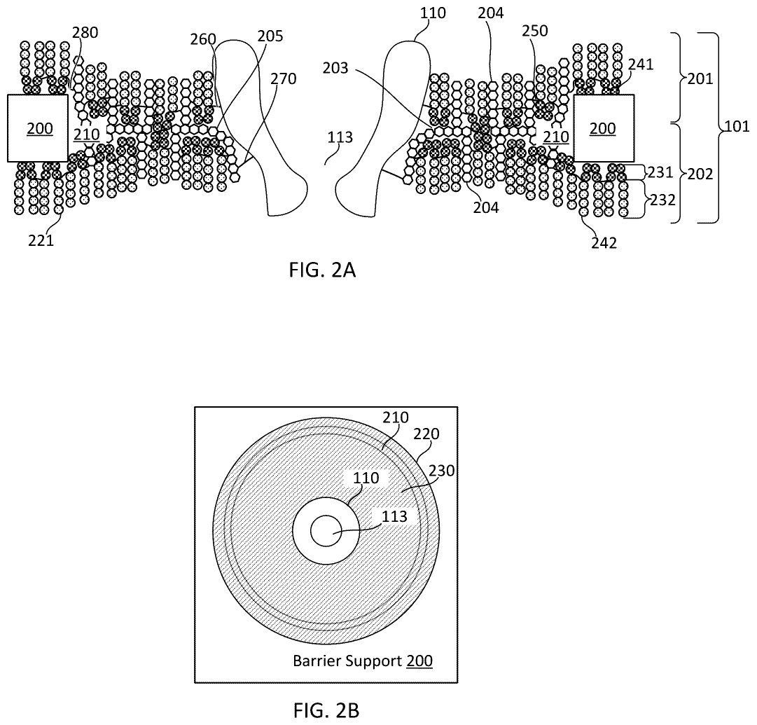

FIG. 1 schematically illustrates a cross-sectional view of an example nanopore composition and device 100 including a polymeric barrier. Device 100 includes fluidic well 100′ including barrier 101, such as a polymeric barrier, having first (trans) side 111 and second (cis) side 112, first fluid 120 within fluidic well 100′ and in contact with first side 111 of the barrier, and second fluid 120′ within the fluidic well and in contact with the second side 112 of the barrier. Barrier 101 may have any suitable structure that normally inhibits passage of molecules from one side of the barrier to the other side of the barrier, e.g., that normally inhibits contact between fluid 120 and fluid 120′. Illustratively, barrier 101 may include a polymeric barrier, which may include a diblock or triblock copolymer and may have a structure such as described in greater detail below with reference to FIGS. 2A-2B, 3A-3F, 4A-4D, 5A-5D, 6A-6B, 7A-7B, 8A-8B, 9A-9B, 10A-10B, 11A-11C, 12, 13A-13C, 14-17, 18A-18C, 20A-20B, 21A-21B, 22A-22B, 23A-23B, and 24-27.

First fluid 120 may have a first composition including a first concentration of a salt 160, which salt may be represented for simplicity as positive ions although it will be appreciated that counterions also may be present. Second fluid 120′ may have a second composition including a second concentration of the salt 160 that may be the same as, or different, than the first concentration. Any suitable salt or salts 160 may be used in first and second fluids 120, 120′, e.g., ranging from common salts to ionic crystals, metal complexes, ionic liquids, or even water-soluble organic ions. For example, the salt may include any suitable combination of cations (such as, but not limited to, H, Li, Na, K, NH₄, Ag, Ca, Ba, and/or Mg) with any suitable combination of anions (such as, but not limited to, OH, Cl, Br, I, NO₃, ClO₄, F, SO₄, and/or CO₃²⁻ . . . ). In one nonlimiting example, the salt includes potassium chloride (KCl). It will also be appreciated that the first and second fluids may, in some embodiments, include any suitable combination of other solutes. Illustratively, first and second fluids 120, 120′ may include an aqueous buffer (such as N-(2-hydroxyethyl) piperazine-N′-2-ethanesulfonic acid (HEPES), commercially available from Fisher BioReagents).

Still referring to FIG. 1 device 100 further may include nanopore 110 disposed within barrier 101 and providing aperture 113 fluidically coupling first side 111 to second side 112. As such, aperture 113 of nanopore 110 may provide a pathway for fluid 120 and/or fluid 120′ (e.g., salt 160) to flow through barrier 101. Nanopore 110 may include a solid-state nanopore, a biological nanopore (e.g., MspA such as illustrated in FIG. 1), or a biological and solid-state hybrid nanopore. Nonlimiting examples and properties of barriers and nanopores are described elsewhere herein, as well as in U.S. Pat. No. 9,708,655, the entire contents of which are incorporated by reference herein. In a manner such as illustrated in FIG. 1, device 100 may, in some embodiments, include first electrode 102 in contact with first fluid 120, second electrode 103 in contact with second fluid 120′, and circuitry 180 in operable communication with the first and second electrodes and configured to detect changes in an electrical characteristic of the aperture. Such changes may, for example, be responsive to any suitable stimulus. Indeed, it will be appreciated that the present methods, compositions, and devices may be used in any suitable application or context, including any suitable method or device for sequencing, e.g., polynucleotide sequencing.

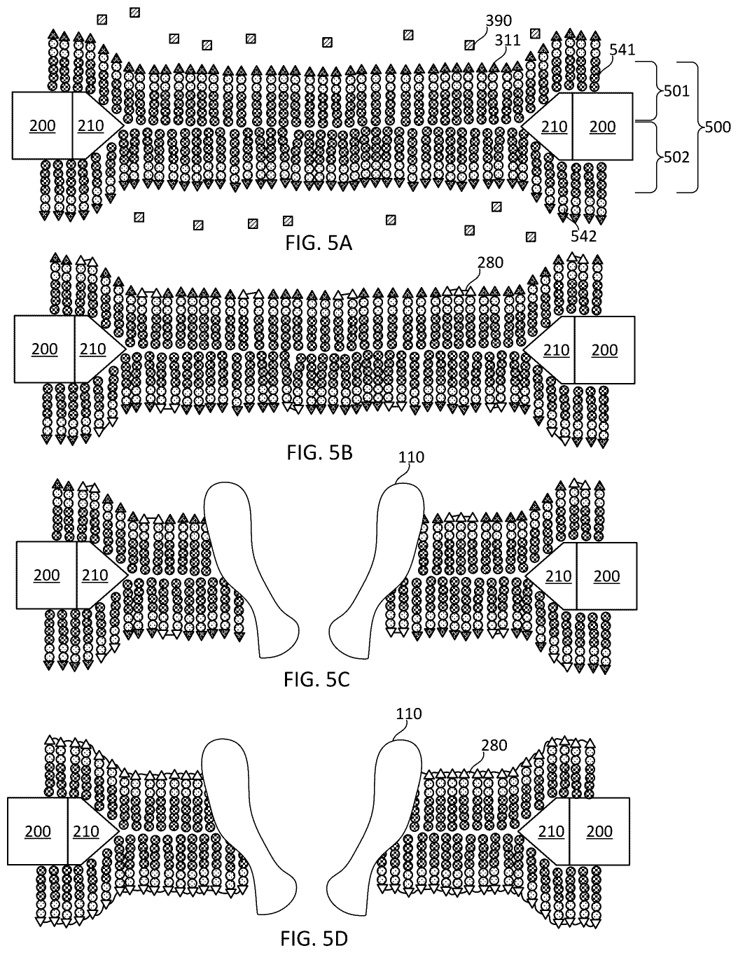

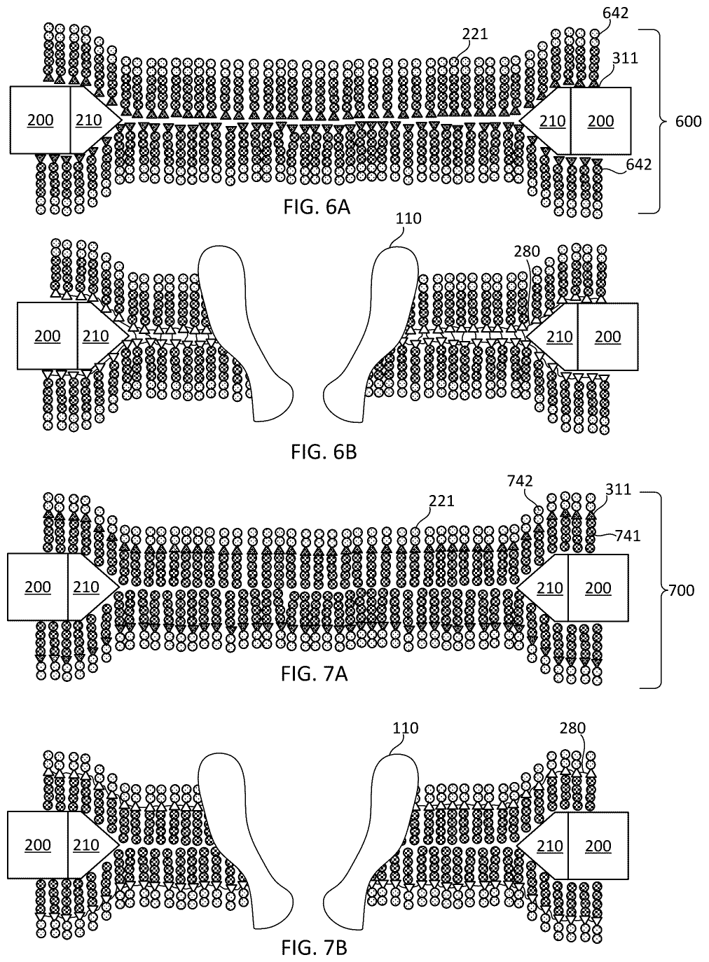

In some examples, polymeric barrier 101 between first and second fluids 120, 120′ includes a block copolymer. For example, FIGS. 2A-2B schematically illustrate plan and cross-sectional views of further details of the nanopore composition and device of FIG. 1. As illustrated in FIG. 2A, barrier 101 may include first layer 201 including a first plurality of amphiphilic molecules 221 and second layer 202 including a second plurality of the amphiphilic molecules 221 contacting the first plurality of amphiphilic molecules. In the nonlimiting example illustrated in FIG. 2A, the copolymer is a triblock copolymer (ABA), such that each molecule 221 includes a hydrophobic “B” block 231 (within which circles 241 with darker fill represent hydrophobic monomers) coupled to first and second hydrophilic “A” blocks 232 (within which circles 242 with lighter fill represent hydrophilic monomers) coupled directly or indirectly thereto. In other examples such as will be described with reference to FIGS. 5A-5B, 8A-8B, and 13, the copolymer instead may include an ABA triblock copolymer. In still other examples such as will be described with reference to FIGS. 6A-6B, 9A-9B, 11A-11B, and 14A-14B, the copolymer instead may include a BAB triblock copolymer.

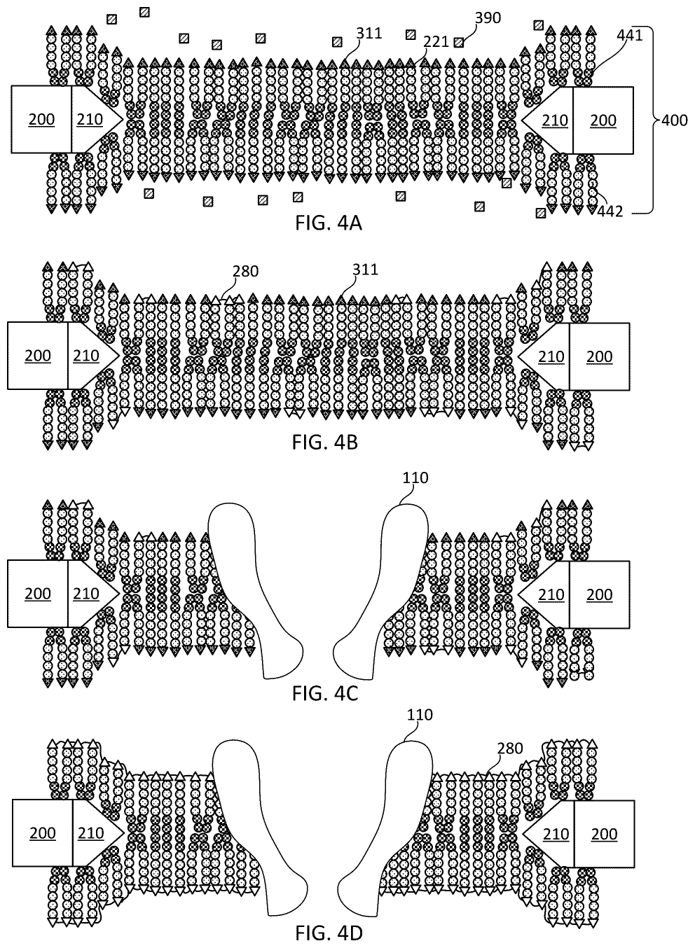

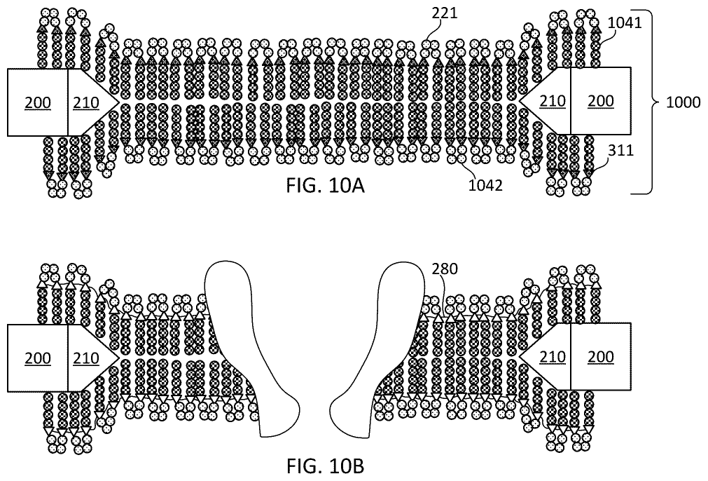

At least some amphiphilic molecules 221 of first layer 201 are crosslinked to one another, and at least some amphiphilic molecules 221 of second layer 202 are crosslinked to one another. In the example illustrated in FIG. 2A, the amphiphilic molecules 221 are cross-linked to one another by bonds 280 which are formed at an interface between the A and B blocks. As such, bonds 280 may strengthen and stabilize the barrier, resulting in improved performance and durability. Alternative configurations of barriers that may be formed that include bonds 280 at an interface between the A and B blocks are described with reference to FIGS. 7A-7B and 10A-10B. Such cross-linking bonds 280 may be formed in any other suitable location(s) within the barrier. For example, in a manner such as described with reference to FIGS. 6A-6B and 9A-9B, hydrophobic blocks 231 may be cross-linked by bonds which are formed at the hydrophobic blocks. Or, for example, in a manner such as described with reference to FIGS. 4A-4D, 5A-5D, and 8A-8B, the hydrophilic blocks 232 may be cross-linked by bonds which are formed at the hydrophilic blocks.

In the example illustrated in FIGS. 2A-2B, barrier 101 may be suspended using a barrier support 200 defining aperture 230. For example, barrier support 200 may include a substrate having an aperture 230 defined therethrough, e.g., a substantially circular aperture, or an aperture having another shape. Additionally, or alternatively, the barrier support may include one or more features of a well in which the nanopore device is formed, such as a lip or ledge on either side of the well. Nonlimiting examples of materials which may be included in a barrier support are provided further above. An annulus 210 including hydrophobic (non-polar) solvent, and which also may include polymer chains and/or other compound(s), may adhere to barrier support 200 and may support a portion of barrier 101, e.g., may be located within barrier 101 (here, between layer 201 and layer 202). Additionally, annulus 210 may taper inwards in a manner such as illustrated in FIG. 2A. An outer portion of the molecules 221 of barrier 101 may be disposed on support 200 (e.g., the portion extending between aperture 230 and barrier periphery 220), while an inner portion of the molecules may form a freestanding portion of barrier 101 (e.g., the portion within aperture 210, a part of which is supported by annulus 210). Note that although the overall assembly of molecules which are cross-linked to one another may itself be considered to form a larger molecule (e.g., a molecule which partially or substantially spans the aperture 230 of the barrier support 220 in FIGS. 2A-2B), the components of such larger molecule also may be referred to herein as molecules so as to facilitate discussion of such components.

In some embodiments, barrier 101 may further include polymer 203 which is in addition to, and has a different composition than, amphiphilic molecules 221. For example, as illustrated in FIG. 2A, at least a portion 204 of polymer 203 may be intercalated between amphiphilic molecules 221 of the at least one layer of barrier 101. Additionally, or alternatively, in examples such as illustrated in FIG. 2A in which the barrier 101 includes first layer 201 and second layer 202, at least portion 205 of polymer 203 is disposed between the first layer 201 and the second layer 202. While at least some of amphiphilic molecules 221 may be crosslinked to one another in a manner via bonds 280 such as described above and elsewhere herein, in some embodiments at least some of amphiphilic molecules 221 also, or alternatively, may be crosslinked to polymer 203. For example, amphiphilic molecules 221 may, in some embodiments, be coupled to polymer 203 via bonds 250. In examples such as illustrated in FIGS. 2A-2B, polymer 203 may form overhanging annulus 210 around aperture 200. In some examples, polymer 203 may substantially cover the aperture, e.g., other than the location at which nanopore 110 is located. Nonlimiting examples of operations and materials for forming polymer 203 are provided elsewhere herein.

Additionally, or alternatively, nanopore 110 may in some embodiments be coupled to amphiphilic molecules 221 via bonds 260. Additionally, or alternatively, nanopore 110 may in some embodiments be coupled to polymer 230 via bonds 270. Nonlimiting examples of operations for forming bonds 260 and/or bonds 270 are provided elsewhere herein.

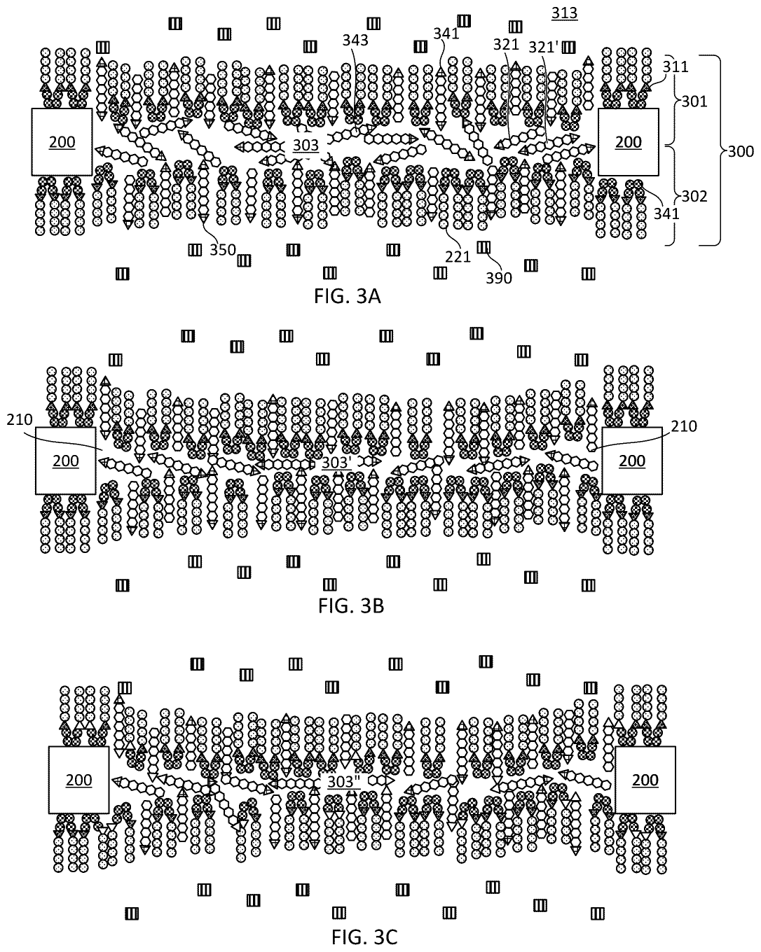

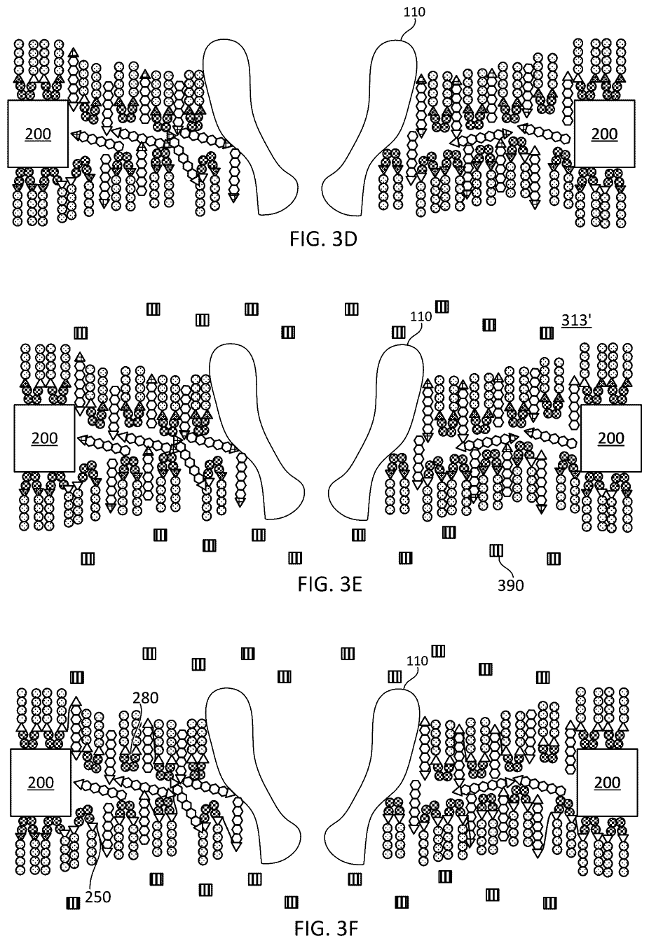

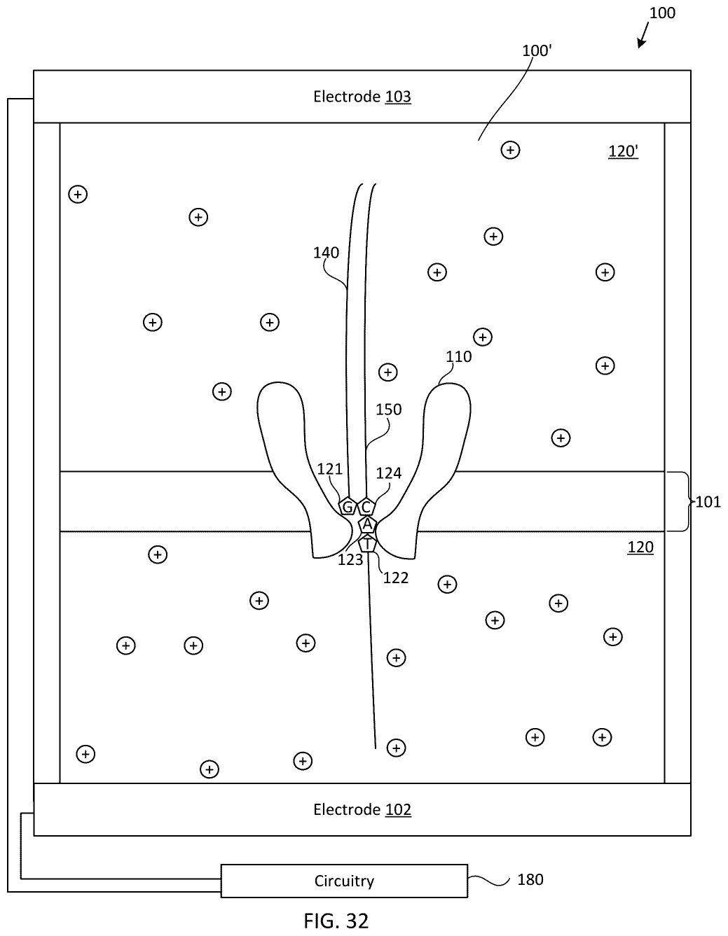

Barrier 101 may be stabilized, and nanopore 110 may be inserted into the freestanding portion of barrier 101, e.g., using operations such as now will be described with reference to FIGS. 3A-3F, 4A-4D, 5A-5D, 6A-6B, 7A-7B, 8A-8B, 9A-9B, 10A-10B, 11A-11C, 12, 13A-13C, 14-17, 18A-18C, 20A-20B, 21A-21B, 22A-22B, 23A-23B, and 24-27. Although FIGS. 2A-2B illustrate nanopore 110 within barrier 101, it should be understood that the nanopore may be omitted, and that barrier 101 may be used for any suitable purpose. More generally, it should be appreciated that while the barriers described herein are particularly suitable for use with nanopores (e.g., for nanopore sequencing such as described with reference to FIGS. 32-36), the present barriers need not necessarily have nanopores inserted therein.

FIGS. 3A-3F schematically illustrate example operations for forming a barrier including a nanopore and cross-linked amphiphilic molecules. As illustrated in FIG. 3A, barrier 300 may be configured, in some regards, similarly as barrier 101 described with reference to FIGS. 2A-2B, e.g., may include at least one layer including amphiphilic molecules. In the nonlimiting example illustrated in FIG. 3A, the barrier may include layer 301 including a first plurality of amphiphilic molecules 221 and layer 302 including a second plurality of amphiphilic molecules 221. However, the amphiphilic molecules in barrier 300 have not yet been crosslinked. Instead, the amphiphilic molecules of layer 301 (and in some embodiments also of layer 302, if present) may include reactive moieties 311. Reactive moieties 311 may be reacted with one another in such a manner as to partially cross-link the amphiphilic molecules 221 with one another before inserting a nanopore into barrier 300, and then to further cross-link the amphiphilic molecules 221 with one another after inserting the nanopore into barrier 300.

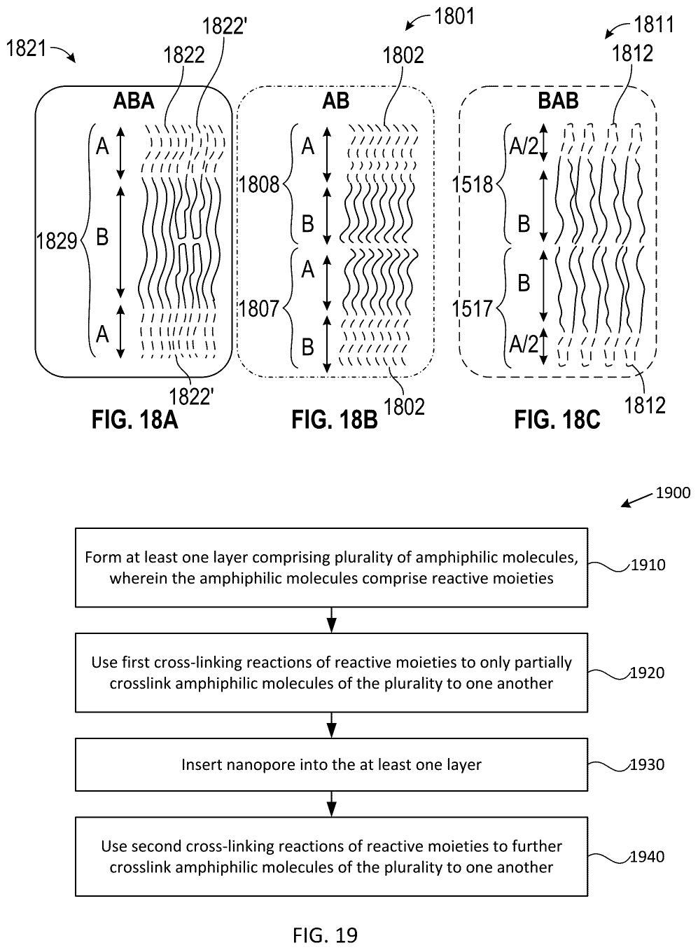

In examples such as illustrated in FIG. 3A, the amphiphilic molecules 221 include molecules of a triblock ABA copolymer which are oriented such that the hydrophobic “B” sections of the ABA diblock copolymer are oriented towards each other and disposed within the barrier, while the hydrophilic “A” sections form the outer surfaces of the barrier. Each individual ABA molecule may be in one of two arrangements. For example, in a manner such as described with reference to FIG. 18A and as shown in FIG. 4A, certain of the ABA molecules may extend through the layer in a linear fashion, with an “A” section on each side of the barrier and the “B” section in the middle of the barrier. Or, for example, in a manner such as illustrated in FIGS. 3A, 4A, and 18A, certain of the ABA molecules may extend to the middle of the barrier and then fold back on themselves, so that both “A” sections are on the same side of the barrier and the “B” section is in the middle of the barrier. Accordingly, in some examples, barrier 300 may be considered to be partially a single layer and partially a bilayer. In other examples (not specifically illustrated) in which barrier 300 substantially includes molecules 221 which extend through the barrier in linear fashion, barrier 300 may substantially be a monolayer. In still other examples (such as illustrated in FIG. 3A) in which barrier 300 substantially includes molecules 221 which extend to approximately the middle of the barrier and then fold back on themselves, barrier 300 may substantially be a bilayer. In the non-limiting example illustrated in FIG. 3A, reactive moieties 311 are located an interface between hydrophilic “A” sections 332 and hydrophobic “B” sections 331.

Barrier 300 may be supported by barrier support 200 having aperture 230 such as described with reference to FIGS. 2A-2B. Suitable methods of forming suspended barriers are known in the art, such as “painting”, e.g., brush painting (manual), mechanical painting (e.g., using stirring bar), and bubble painting (e.g., using flow through the device). In such methods, a thin layer of hydrophobic liquid between aqueous solutions may be used to cause preferential orientation of the amphiphilic molecules' hydrophobic moieties to the interior of the barrier, and preferential orientation of the amphiphilic molecules' hydrophilic moieties to the outside of the barrier so as to form a barrier which is suspended across aperture 230 using barrier support 200.

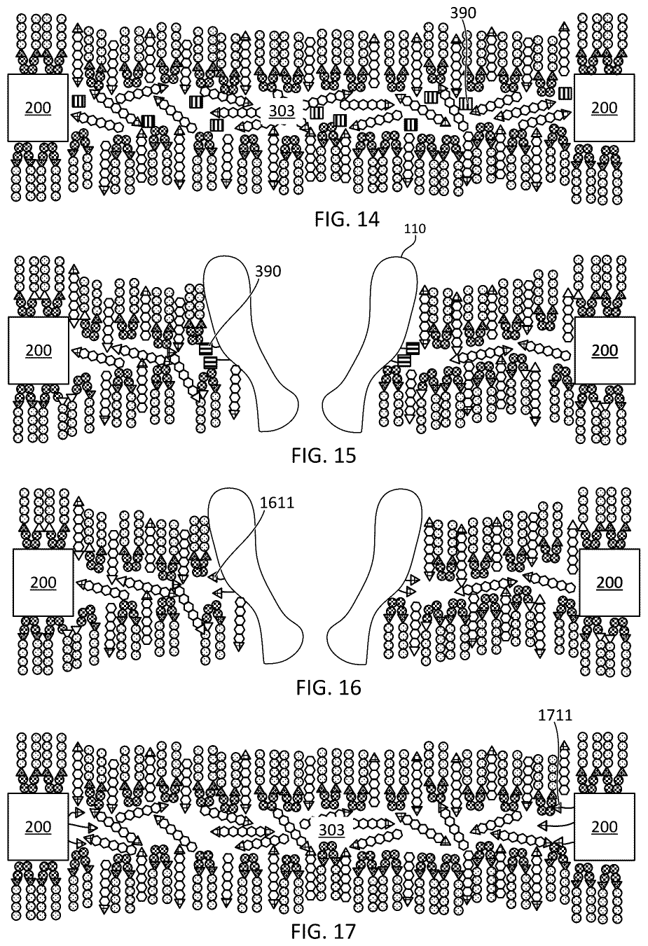

Barrier 300 may be formed using aqueous liquid 313 (e.g., an aqueous buffer solution) which is substantially on the outside of the barrier, and a hydrophobic liquid 303 which becomes disposed within the barrier. Hydrophobic liquid 303 may include an organic solvent such as such as an alkane, e.g., octane, decane, dodecane, or hexadecane. Alternatively, in some examples provided herein, hydrophobic liquid 303 may include hydrophobic, polymerizable monomers. In specific examples such as illustrated in FIG. 3A, hydrophobic liquid 303 may consist essentially of hydrophobic, polymerizable monomers 321, 321′, e.g., may include less than about 10 wt %, less than about 5 wt %, less than about 2 wt %, or less than about 1 wt % of compound(s) other than for the polymerizable monomers, such as an initiator that initiates polymerization of the monomers in a manner such as described with reference to FIG. 14. That is, monomers 321, 321′ are liquid at the conditions under which barrier 300 is formed, and sufficiently dissolve the amphiphilic molecules' hydrophobic moieties to facilitate formation of barrier 300. As such, no other solvent besides the monomers need be included in liquid 303 and the monomers themselves may be used to cause preferential orientation of the amphiphilic molecules' hydrophobic moieties to the interior of the barrier, and preferential orientation of the amphiphilic molecules' hydrophilic moieties to the outside of the barrier so as to form bilayer barrier 300 such as illustrated in FIG. 3A. In some embodiments, hydrophobic liquid 303 also may include one or more additives in an amount of less than about 10 wt % that may help adjust the properties of the polymer being generated, such as a plasticizer and/or a relatively small amount of a thinner solvent e.g., less than about 10 wt %, less than about 5 wt %, less than about 2 wt %, or less than about 1 wt % of a hydrophobic solvent.

In some embodiments, during assembly of barrier 300, monomers 321, 321′ may become intercalated between the amphiphilic molecules. In the nonlimiting example illustrated in FIG. 3A, a first portion 341 of the monomers 321, 321′ become intercalated between amphiphilic molecules 221 of the first layer 301 and a second portion 342 of the monomers 321, 321′ become intercalated between amphiphilic molecules 221 of the second layer 302. Illustratively, hydrophobic attraction of hydrophobic monomers 321, 321′ to the hydrophobic moieties of amphiphilic molecules 221 may cause the monomers to insert between such hydrophobic moieties. A third portion 343 of the monomers 321, 321′ may be located approximately between layers 301 and 302.

In nonlimiting examples including monomers 321, 321′, such monomers may be capable of reacting with one another so as to form a polymer, e.g., polymer 203 described with reference to FIGS. 2A-2B. Additionally, or alternatively, monomers 321, 321′ may be capable of reacting with moieties 311 of amphiphilic molecules 221 so as to couple polymer 203 to the amphiphilic molecules 221 via bonds 250 in a manner such as described with reference to FIGS. 2A-2B. In some examples, at least some of the monomers within liquid 303 may include a single reactive moiety 350, such monomers being referred to as 321 for case of distinction from monomers that include two reactive moieties. Additionally, or alternatively, at least some of the monomers within liquid 303 may include two or more reactive moieties 350, such monomers being referred to as 321′ for ease of distinction from monomers that include a single reactive moiety. Moieties 350 of monomers 321, 321′ may be of the same type as moieties 311 of amphiphilic molecules 221; as such, moieties 350 may react with moieties 311 to form bonds 250 and/or with other moieties 350 to form polymer 203. Alternatively, moieties 350 of monomers 321, 321′ may be of a different type than moieties 311 of amphiphilic molecules 221. In some such examples, moieties 350 may react with moieties 311 to form bonds 250 and/or with other moieties 350 to form polymer 203. In other such examples, moieties 350 may not react with moieties 311, and may react with other moieties 350 to form polymer 203. Nonlimiting examples of moieties 311 and 350 are provided elsewhere herein.

Note that for any monomers 321, 321′ that intercalate between amphiphilic molecules 221, in some examples the reactive group 350 may oriented toward the outer surface of barrier 300, or may be oriented toward the inside of barrier 300. In the nonlimiting example illustrated in FIG. 3A, liquid 303 includes a mixture of monomers 321 and 321′. The relative proportion of monomers 321 to 321′ within liquid 303 may be selected so as to provide an extent of polymerization within the barrier that provides a suitable level of stability. Illustratively, the relative weight percent of monomers 321 relative to monomers 321′ may be in the range of about 1 wt. % to about 50 wt. %, e.g., about 10 wt. % to about 50 wt. %, e.g., about 20 wt. % to about 50 wt. %.

In some examples, as illustrated in FIG. 3A, fluid 313 may include at least one initiator, e.g., initiator 390. Initiator 390 may be selected so as to be chemically reactive with reactive moieties 311, e.g., so as to form products in which amphiphilic molecules 221 are cross-linked to one another, such as via polymerization. Additionally, or alternatively, initiator 390 may be selected so as to be chemically reactive with reactive moieties 350, e.g., so as to form products in which monomers 321, 321′ are cross-linked to one another, such as via polymerization. Additionally, or alternatively, initiator 390 may be selected so as to be chemically reactive with reactive moieties 350 and with moieties 311, e.g., so as to form products in which monomers 321 and/or 321′ are cross-linked to amphiphilic molecules 221, such as via polymerization. Additionally, or alternatively, fluid 313 may include a first initiator that is selected so as to be chemically reactive with reactive moieties 311, e.g., so as to form products in which amphiphilic molecules 221 are cross-linked to one another, and a second initiator that is different than the first initiator and is selected so as to be chemically reactive with reactive moieties 350, e.g., so as to form products in which monomers 321, 321′ are cross-linked to one another. In other examples, the initiator may be omitted and reactive moieties 311 and/or 350 may react directly with one another without use of an initiator.

In a manner such as illustrated in FIG. 3A, barrier 300 initially may be swelled by hydrophobic liquid 303, such that monomers 321, 321′ (if used) may move fluidically within barrier 300. Before polymerizing monomers 321, 321′, barrier 300 may be thinned in a manner such as illustrated in FIG. 3B, resulting in layer 303′ having a reduced amount of the hydrophobic liquid within the barrier. For example, the viscosity of the hydrophobic liquid may be selected such that hydrostatic pressure on the barrier overcomes the resistance in the hydrophobic liquid. As a result of such thinning, annulus 210 including the hydrophobic liquid, denoted 303′ in FIG. 3B, may be generated which is adjacent to support 200 and includes a greater thickness of hydrophobic liquid 303′ than does the middle of the membrane.

Cross-linking reactions of reactive moieties within barrier 300 then may be used to only partially crosslink amphiphilic molecules 221 to one another. For example, FIG. 3C illustrates the products of polymerization reactions between amphiphilic molecules 221, in which bonds 280 are formed between only some of reactive moieties 311 (the fill of the reacted moieties is changed from crosshatched to white to indicate that such moieties have reacted and are no longer available for reaction, with the bonds which are formed being represented by moieties touching one another). In some embodiments, in the example illustrated in FIG. 3C, monomers 321, 321′ may be only partially polymerized to form polymer 303″ disposed within and between the first and second layers. For example, reactive groups 350 of monomers 321 may react with the reactive groups of other monomers 321 or with reactive groups of monomers 321′ in such a manner as to form covalent bonds, as is intended to be indicated in FIG. 3C by the change in fill of the triangles representing reactive groups 350 and by the moieties touching one another. As an additional option, or as an alternative option, monomers 321, 321′ may be only partially reacted with amphiphilic molecules 221. For example, reactive groups 350 of monomers 321 and/or of monomers 321′ may react with reactive moieties 311 of amphiphilic molecules 221 in such a manner as to form covalent bonds, as is intended to be indicated in FIG. 3C by the change in fill of the triangles representing reactive groups 350 and by the moieties touching one another.

In some examples, following partial cross-linking of amphiphilic molecules 221, nanopore 110 may be inserted into the barrier in a manner such as illustrated in FIG. 3D. Nonlimiting examples of techniques for inserting nanopore 110 into the barrier, whether before or after crosslinking, include electroporation, pipette pump cycle, and detergent assisted nanopore insertion. Tools for forming barriers using synthetic polymers and inserting nanopores in the barriers are commercially available, such as the Orbit 16 TC platform available from Nanion Technologies Inc. (California, USA).

After the nanopore is inserted, cross-linking reactions of reactive moieties within barrier 300 then may be used to further crosslink amphiphilic molecules 221 to one another. For example, as shown in FIG. 3E, the partially polymerized barrier with nanopore 110 inserted therein may be contacted with fluid 313′ including at least one initiator, e.g., initiator 390 which may be of the same type as described with reference to FIGS. 3A-3C. In a manner such as described with reference to FIGS. 3A-3C, the initiator(s) within fluid 313′ may be selected so as to be chemically reactive with reactive moieties 311 and/or with reactive moieties 350, e.g., so as to form products in which amphiphilic molecules 321 are cross-linked to one another, and/or in which monomers 321, 321′ are cross-linked to one another, and/or in which monomers 321 and/or 321′ are cross-linked to amphiphilic molecules 221, such as via polymerization.

FIG. 3F illustrates the products of the additional polymerization reactions between amphiphilic molecules 221, in which additional bonds 280 are formed between reactive moieties 311 (the fill of the reacted moieties is changed from crosshatched to white to indicate that such moieties have reacted and are no longer available for reaction, with the bonds which are formed being represented by moieties touching one another or by lines drawn between the moieties). In some embodiments, in the example illustrated in FIG. 3F, additional monomers 321, 321′ may be polymerized to form polymer 303″ disposed within and between the first and second layers. For example, reactive groups 350 of monomers 321 may react with the reactive groups of other monomers 321 or with reactive groups of monomers 321′ in such a manner as to form covalent bonds, as is intended to be indicated in FIG. 3F by the change in fill of the triangles representing reactive groups 350 and by the moieties touching one another. As an additional option, or as an alternative option, monomers 321, 321′ may be reacted with amphiphilic molecules 221 to form additional bonds 250. For example, reactive groups 350 of monomers 321 and/or of monomers 321′ may react with reactive moieties 311 of amphiphilic molecules 221 in such a manner as to form covalent bonds 250, as is intended to be indicated in FIG. 3C by the change in fill of the triangles representing reactive groups 350 and by the moieties touching one another or by lines drawn between the moieties.

Although FIG. 3F may suggest that reactive moieties 311 may be cross-linked to one or two other moieties 311 or 350 via respective bonds 280 or 250, and that reactive moieties 350 may be cross-linked to one or two other moieties 311 or 350 via respective bonds, it will be appreciated that each reactive moiety 311, 350 may form bonds with any suitable number of other reactive moieties, e.g., one, two, three, or more than three other reactive moieties. The relative proportion of such products may be controlled, e.g., through the type(s) of reactive moieties used, the respective number(s) of reactive moieties used, the type(s) of initiator used, reaction time, and the reaction conditions, so as to control the amount of cross-linking provided using reaction between the reactive moieties 311 of molecules 221 and/or between the reactive moieties 350 of monomers 321, 321′ (if used). Cross-linking also may be controlled through coupling strategies. For example, thiol-ene or thiol-yne reactions may be used that are based on generating radicals and can be controlled with type and concentration of initiator. Cross-linking triggered by a reducing agent alternatively may be used and concentration and type of reducing agent can be used to control the reaction. Alternatively, an initiator free strategy may be used which uses UV light to trigger cross-linking, and the reaction can be controlled by UV dose (irradiance, wavelength and time); in such examples, the barrier may be enclosed within a structure which is at least partially transparent to the UV light, and the light transmitted through the structure to cause cross-linking of the barrier therein. Other strategies may use two amphiphilic polymers with different reactive moieties, in which the ratio between the amphiphilic polymers may be selected to achieve substantially full cross-linking. Depending on the strategy, this substantially full cross-linking can be achieved with an example ratio of 1:1 or 2:1. If lower degree of cross-linking is desired, ratios can be tuned to achieve partial cross-linking. Additionally, in some examples, the amount of cross-linking may be controlled by mixing amphiphilic molecules 221 in suitable proportion with other amphiphilic molecules that do not include reactive moieties 311, or that include different reactive moieties, and/or that have a different architecture (e.g., AB can be mixed with ABA and/or BAB; ABA can be mixed with AB and/or BAB; and/or BAB can be mixed with AB and/or ABA).

Although FIGS. 3A-3F illustrate operations for cross-linking the hydrophilic blocks of a triblock ABA copolymer having reactive moieties at the A-B interface, it will be appreciated that such operations similarly may be used to cross-link other portions of a triblock copolymer or to cross-link other types of amphiphilic molecules, such as other types of polymers. For example, FIGS. 4A-4D schematically illustrate example operations for forming an alternative barrier including a nanopore and crosslinked amphiphilic molecules. FIG. 4A illustrates suspended barrier 400 including molecules 221 of an ABA triblock copolymer including hydrophobic “B” sections 441 coupled to and between hydrophilic “A” sections 442 in a manner similar to that described with reference to FIGS. 3A-3F. Barrier 400 may be suspended using barrier support 200 and, in some embodiments, annulus 210 in a manner similar to that described with reference to FIGS. 2A-2B and 3A-3F. In a manner such as illustrated in FIG. 4A and as described with reference to FIG. 18A, each individual ABA molecule 221 may be in one of two arrangements, e.g., may extend through the layer in a linear fashion or may extend to the middle of the barrier and then fold back on themselves. In the nonlimiting example illustrated in FIG. 4A, reactive moieties 311 may be coupled to hydrophilic sections 442, e.g., to the terminal hydrophilic monomer of such section. Initiator 390 may be used to partially cross-link reactive moieties 311 with one another in a manner similar to that described with reference to FIGS. 3B-3C so as to form bonds 280 illustrated in FIG. 4B. In a manner such as described with reference to FIG. 3D, and as illustrated in FIG. 4C, nanopore 110 may be inserted into the barrier after the partial cross-linking. In a manner such as described with reference to FIG. 3F, after inserting the nanopore, cross-linking of reactive moieties 311 may be used to further crosslink amphiphilic molecules 221 to one another, e.g., forming additional bonds 280 illustrated in FIG. 4D. Although not specifically illustrated in FIGS. 4A-4D, it should be understood that the barrier also in some embodiments may include monomers 321, 321′ which may be reacted in a manner such as described with reference to FIGS. 3A-3F to form polymer 203 and/or to form bonds 250 with molecules 221.

Other types of block copolymers suitably may be used. For example, FIGS. 5A-5D schematically illustrate example operations for forming an alternative barrier including a nanopore and crosslinked amphiphilic molecules. FIG. 5A illustrates suspended barrier 500 including molecules 221 of an AB diblock copolymer which are oriented such that the hydrophobic “B” sections of the AB diblock copolymer are oriented towards each other and disposed within the barrier, while the hydrophilic “A” sections form the outer surfaces of the barrier. In the non-limiting example illustrated in FIG. 5A, hydrophilic “A” sections 542 may include reactive moieties 311, e.g., coupled to the terminal hydrophilic monomer. Initiator 390 may be used to partially cross-link reactive moieties 311 with one another in a manner similar to that described with reference to FIGS. 3B-3C so as to form bonds 280 illustrated in FIG. 5B. In a manner such as described with reference to FIG. 3D, and as illustrated in FIG. 5C, nanopore 110 may be inserted into the barrier after the partial cross-linking. In a manner such as described with reference to FIG. 3F, after inserting the nanopore, cross-linking of reactive moieties 311 may be used to further crosslink amphiphilic molecules 221 to one another, e.g., forming additional bonds 280 illustrated in FIG. 5D. Although not specifically illustrated in FIGS. 5A-5D, it should be understood that the barrier in some embodiments also may include monomers 321, 321′ which may be reacted in a manner such as described with reference to FIGS. 3A-3F to form polymer 203 and/or to form bonds 250 with molecules 221.

In other examples, the reactive moiety may be located at the end of the hydrophobic B block. Illustratively, FIGS. 6A-6B schematically illustrate example operations for forming another alternative barrier including a nanopore and crosslinked amphiphilic molecules. Suspended barrier 600 illustrated in FIG. 6A includes AB diblock copolymer molecules 221 in which reactive moiety 311 is located at hydrophobic block 641, e.g., is coupled to the terminal monomer of the hydrophobic block. Barrier 600 may be suspended using barrier support 200 and, in some embodiments, annulus 210 in a manner similar to that described with reference to FIGS. 2A-2B. In a manner such as described with reference to FIGS. 3B-3F, cross-linking reactions of reactive moieties 311 may be used so as to only partially cross-link amphiphilic molecules 221 to one another via bonds 280, then nanopore 110 may be inserted into the barrier, and then cross-linking of reactive moieties 311 may be used to further crosslink amphiphilic molecules 221 to one another, e.g., forming additional bonds 280 illustrated in FIG. 6B. Although not specifically illustrated in FIGS. 6A-6B, it should be understood that the barrier in some embodiments also may include monomers 321, 321′ which may be reacted in a manner such as described with reference to FIGS. 3A-3F to form polymer 203 and/or to form bonds 250 with molecules 221.

As noted elsewhere herein, it will be appreciated that reactive moieties 311 may be provided at any suitable locations within the barrier and reacted so as to cross-link the amphiphilic molecules at such locations. For example, the reactive moiety may be located at an A-B interface. Illustratively, FIGS. 7A-7B schematically illustrate example operations for forming another alternative barrier including a nanopore and crosslinked amphiphilic molecules. Suspended barrier 700 illustrated in FIG. 7A includes AB diblock copolymer molecules 221 in which reactive moiety 311 is located at the A-B interface between hydrophilic block 742 and hydrophobic block 741. Barrier 700 may be suspended using barrier support 200 and, in some embodiments, annulus 210 in a manner similar to that described with reference to FIGS. 2A-2B. In a manner such as described with reference to FIGS. 3B-3F, cross-linking reactions of reactive moieties 311 may be used so as to only partially cross-link amphiphilic molecules 221 to one another via bonds 280, then nanopore 110 may be inserted into the barrier, and then cross-linking of reactive moieties 311 may be used to further crosslink amphiphilic molecules 221 to one another, e.g., forming additional bonds 280 illustrated in FIG. 7B. Although not specifically illustrated in FIGS. 7A-7B, it should be understood that the barrier in some embodiments also may include monomers 321, 321′ which may be reacted in a manner such as described with reference to FIGS. 3A-3F to form polymer 203 and/or to form bonds 250 with molecules 221.

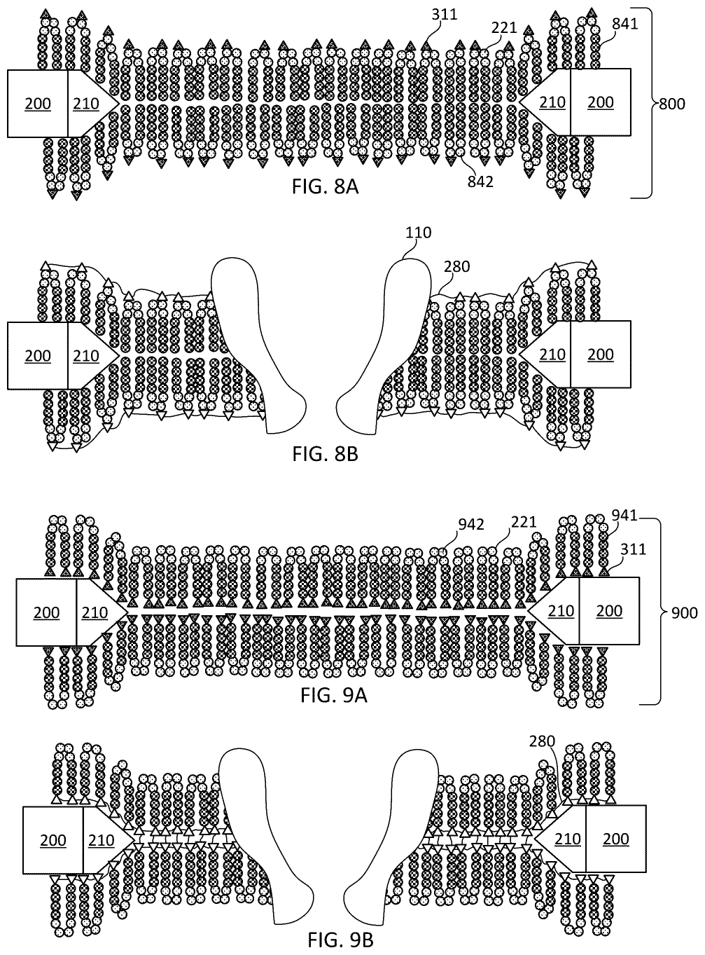

FIGS. 8A-8B schematically illustrate example operations for forming another alternative barrier including a nanopore and crosslinked amphiphilic molecules. FIG. 8A illustrates suspended barrier 800 including molecules 221 of a BAB triblock copolymer including hydrophilic “A” sections 842 coupled to and between hydrophobic “B” sections 841. Barrier 800 may be suspended using barrier support 200 and, in some embodiments, annulus 210 in a manner similar to that described with reference to FIGS. 2A-2B. In this example, barrier 800 may have a bilayer architecture with the “B” sections 641 oriented towards each other. The hydrophobic ends of the BAB molecules generally may located approximately in the middle of barrier 800, the molecules then extend towards either outer surface of the barriers, and then fold back on themselves. As such, both “B” sections are located in the middle of the barrier and the “A” section is on one side or the other of the barrier. Reactive moieties 311 may be coupled to hydrophilic sections 842, e.g., to one or more hydrophilic monomers of such section. In a manner such as described with reference to FIGS. 3B-3F, cross-linking reactions of reactive moieties 311 may be used so as to only partially cross-link amphiphilic molecules 221 to one another via bonds 280, then nanopore 110 may be inserted into the barrier, and then cross-linking of reactive moieties 311 may be used to further crosslink amphiphilic molecules 221 to one another, e.g., forming additional bonds 280 illustrated in FIG. 8B. Although not specifically illustrated in FIGS. 8A-8B, it should be understood that the barrier in some embodiments also may include monomers 321, 321′ which may be reacted in a manner such as described with reference to FIGS. 3A-3F to form polymer 203 and/or to form bonds 250 with molecules 221.

FIGS. 9A-9B schematically illustrate example operations for forming another alternative barrier including a nanopore and crosslinked amphiphilic molecules. Suspended barrier 900 illustrated in FIG. 9A includes BAB triblock copolymer molecules 221 in which reactive moiety 311 is located at hydrophobic block 941, e.g., is coupled to the terminal monomer of the hydrophobic block. In a manner such as described with reference to FIGS. 3B-3F, cross-linking reactions of reactive moieties 311 may be used so as to only partially cross-link amphiphilic molecules 221 to one another via bonds 280, then nanopore 110 may be inserted into the barrier, and then cross-linking of reactive moieties 311 may be used to further crosslink amphiphilic molecules 221 to one another, e.g., forming additional bonds 280 illustrated in FIG. 9B. Although not specifically illustrated in FIGS. 9A-9B, it should be understood that the barrier in some embodiments also may include monomers 321, 321′ which may be reacted in a manner such as described with reference to FIGS. 3A-3F to form polymer 203 and/or to form bonds 250 with molecules 221.

FIGS. 10A-10B schematically illustrate example operations for forming another alternative barrier including a nanopore and crosslinked amphiphilic molecules. Suspended barrier 1000 illustrated in FIG. 10A includes BAB triblock copolymer molecules 221 in which reactive moiety 311 is located at the A-B interface between hydrophilic block 1042 and hydrophobic block 1041. Barrier 1000 may be suspended using barrier support 200 and, in some embodiments, annulus 210 in a manner similar to that described with reference to FIGS. 2A-2B. In a manner such as described with reference to FIGS. 3B-3F, cross-linking reactions of reactive moieties 311 may be used so as to only partially cross-link amphiphilic molecules 221 to one another via bonds 280, then nanopore 110 may be inserted into the barrier, and then cross-linking of reactive moieties 311 may be used to further crosslink amphiphilic molecules 221 to one another, e.g., forming additional bonds 280 illustrated in FIG. 10B. Although not specifically illustrated in FIGS. 10A-10B, it should be understood that the barrier in some embodiments also may include monomers 321, 321′ which may be reacted in a manner such as described with reference to FIGS. 3A-3F to form polymer 203 and/or to form bonds 250 with molecules 221.

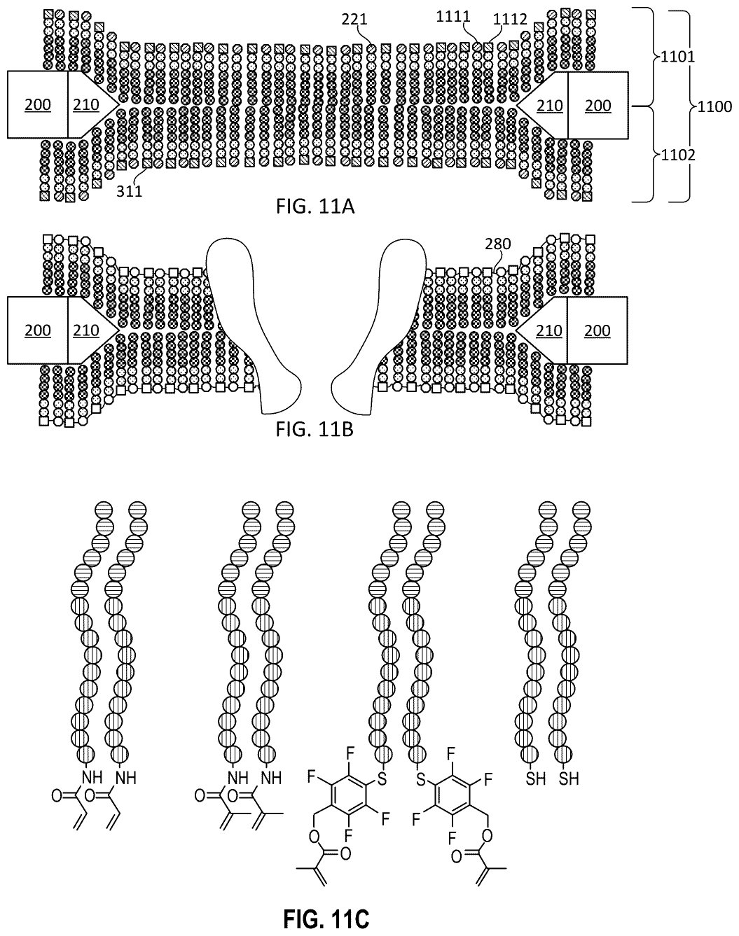

Note that depending on the particular arrangement and proximity of reactive moieties 311 to one another, in various examples provided herein bonds 280 may be located within a particular plane or planes within the barrier. For example, where bonds 280 cross-link the hydrophilic portions of amphiphilic molecules, e.g., such as described with reference to FIGS. 4A-4D, 5A-5D, and 8A-8B, and as will be described further below with reference to FIGS. 11A-11C, one set of the bonds 280 substantially may be located in a first plane providing a first outer surface of the barrier, and another set of the bonds 280 substantially may be located in a second plane providing a second outer surface of the barrier. For example, when the barrier is partially or substantially a bilayer, the bonds 280 of one of the barrier layers substantially may be located in a first plane providing a first outer surface of the barrier, the bonds 280 of the other one of the barrier layers substantially may be located in a second plane providing a second outer surface of the barrier; alternatively, when the barrier is substantially a monolayer, one set of the bonds 280 of that barrier substantially may be located in a first plane providing a first outer surface of the barrier, and another set of the bonds 280 substantially may be located in a second plane providing a second outer surface of the barrier.

Or, for example, where bonds 280 cross-link the hydrophilic-hydrophobic interfaces of amphiphilic molecules, e.g., such as described with reference to FIGS. 2A-2B, 3A-3F, 7A-7B, and 10A-10B, and as will be described further below with reference to FIG. 12, one set of the bonds 280 substantially may be located in a first plane within the barrier, and another set of the bonds 280 substantially may be located in a second plane within barrier. For example, when the barrier is partially or substantially a bilayer, the bonds 280 of one of the barrier layers substantially may be located in a first plane within a first layer of the barrier, the bonds 280 of the other one of the barrier layers substantially may be located in a second plane within a second layer within the barrier; alternatively, when the barrier is substantially a monolayer, one set of the bonds 280 of that barrier substantially may be located in a first plane within the monolayer, and another set of the bonds 280 substantially may be located in a second plane within the monolayer.

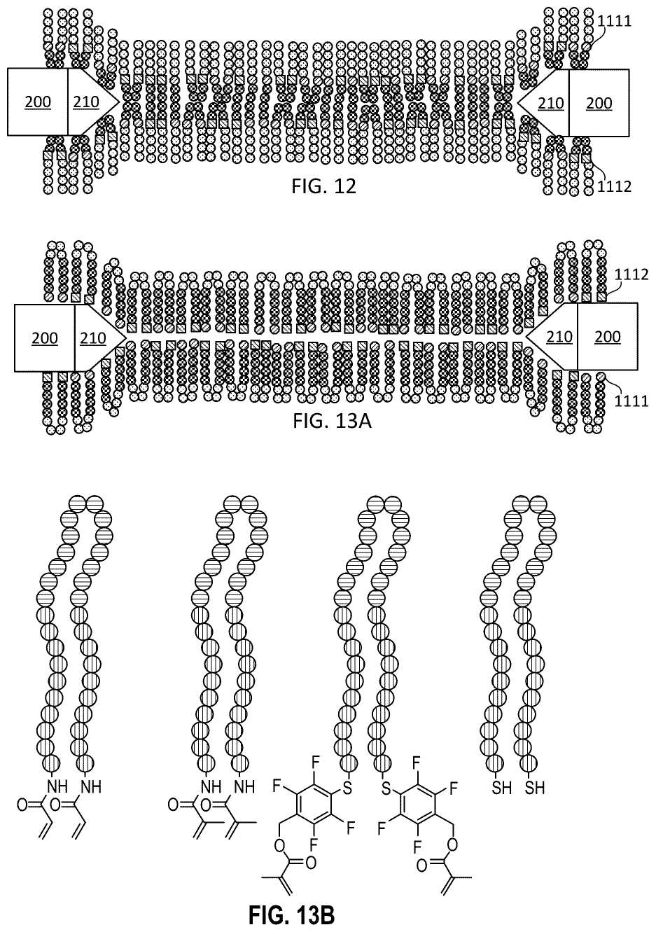

Or, for example, where bonds 280 cross-link hydrophobic portions of amphiphilic molecules, e.g., such as described with reference to FIGS. 6A-6B, 9A-9B, and as will be described further below with reference to FIGS. 13A-13B, the bonds 280 of each of the barrier layers may be located in one or more planes between the two layers. Illustratively, in a manner such as shown in FIGS. 6B and 9B, bonds 280 may be formed between reactive moieties 311 within the plane of the respective layer and/or may be formed between reactive moieties 311 in different planes than one another.

A variety of reactive moieties 311 may be used in polymerization and cross-linking reactions such as described with reference to FIGS. 3A-10B. For example, reactive moieties 311 may be selected from the group consisting of an itaconic moiety, an N-carboxyanhydride moiety, a disulfyl pyridyl moiety, an acrylate moiety, a methacrylate moiety, an acrylamide moiety, a methacrylamide moiety, a styrenic moiety, a maleic moiety, a carboxylic acid moiety, a thiol moiety, an allyl moiety, a vinyl moiety, a propargyl moiety, and a maleimide moiety. In some examples, the polymerization reaction includes a ring-opening polymerization or a step-growth polymerization.

For AB and BAB architectures, there are ways of having a reactive moiety at the end of the B block and those could be crosslinked/polymerized (so the cross-linkages may extend laterally within the barrier). Examples of polymerizable moieties include but are not limited to acrylates or acrylamide derivatives; examples of crosslinkable moieties include but are not limited to thiols and alkenes/alkynes (to generate sulfides), thiols and maleimides (to generate thiosuccinimides), azides and alkynes/BCN/DBCO, thiols and thiols (to generate disulfides), dimethylmaleimide moieties, and the like.

For ABA architecture, there is not per se a ‘free end’ to the B block, however, some B blocks may be flanked with a central reactive moiety. Illustratively, such B blocks can be synthesized as follows: a homo-difunctional initiator containing a third central reactive moiety (such as those described above); the latter may not take part in the polymerization reaction (this can be done either through ensuring orthogonality or by being protected). Such polymerization may generate a telechelic B block that may be terminated in a fashion as to generate reactive ends that can react with the A blocks to generate ABA architecture, while preserving the aforementioned central reactive moiety for later use in the barrier for crosslinking/polymerization purposes. Alternative ways of generating such B blocks include, but not limited to: using heterodifunctional initiators (one functionality intended for the A block coupling, the other one is the initiating moiety) to polymerize B blocks, where the termination step uses a homo-difunctional initiator containing a third central reactive moiety (such as those described above) and 2 reactive moieties that can react with 2 growing B blocks.

For AB, BAB and ABA architectures, reactive moieties may be provided at the AB interface and those could be crosslinked/polymerized (so the cross-linkages may extend laterally within the barrier). Examples of polymerizable moieties include but are not limited itaconic of maleic acid derivatives; examples of crosslinkable moieties include but are not limited to thiols and alkenes/alkynes (to generate sulfides), thiols and thiols (to generate disulfides), dimethylmalcimide moieties, and the like.

For AB and ABA architectures, a reactive moiety may be provided at the end of the A block and those may be crosslinked/polymerized (so the cross-linkages may extend through the outer part of the barrier laterally). Examples of polymerizable moieties include but are not limited to acrylates or acrylamide derivatives; examples of crosslinkable moieties include but are not limited to thiols and alkenes/alkynes (to generate sulfides), thiols and thiols (to generate disulfides), azides and alkynes/BCN/DBCO, dimethylmaleimide moieties, etc.

For BAB architecture, there is not per se a ‘free end’ to the A block, however, A blocks may be flanked with a central reactive moiety. Illustratively, such A blocks may be synthesized as follows: a homo-difunctional initiator including a third central reactive moiety (such as those described above); the latter may not take part in the polymerization reaction (this can be done cither through ensuring orthogonality or by being protected). Such polymerization would generate a telechelic A block that may be terminated in a fashion as to generate reactive ends that can react with the B blocks to generate BAB architecture, while preserving the aforementioned central reactive moiety for later use in the barrier for crosslinking/polymerization purposes. There are alternative ways of generating such A blocks, including but not limited to: using heterodifunctional initiators (one functionality intended for the B block coupling, the other one is the initiating moiety) to polymerize A blocks, where the termination step uses a homo-difunctional initiator containing a third central reactive moiety (such as those described above) and 2 reactive moieties that can react with 2 growing A blocks.

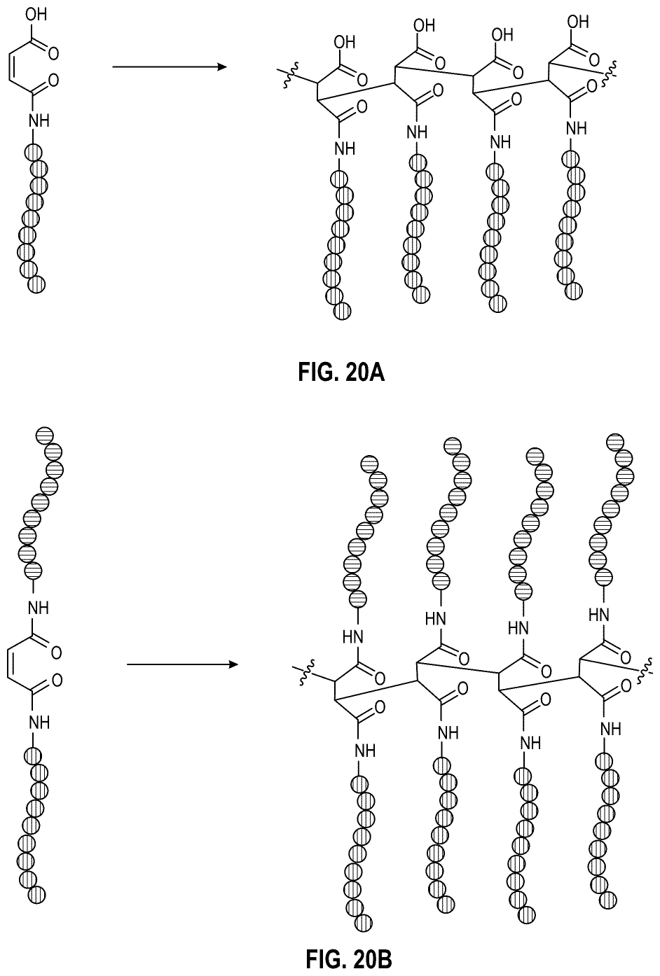

FIG. 20A schematically illustrates an example reaction to form barriers that are crosslinked as described with reference to FIG. 4A-4D, 5A-5D, 6A-6B, 8A-8B, or 9A-9B. As shown in FIG. 20A, the reaction product of the polymerization of the maleic moieties of an example block copolymer includes poly(maleic acid derivative) formed in a plane at the ends of the A blocks of the block copolymer—where in this specific example, the A blocks are formed by a single maleic moiety, but in other implementations, the A block maybe be formed by a hydrophilic polymer with a terminal maleic moiety. Note that although the maleic moieties on the other side of the barrier are not shown, in some examples a similar reaction product may be formed in a plane on the other side of the barrier, e.g., in examples in which both sides of the barrier are contacted with the initiator. In other examples, the reaction product substantially may be formed only at the ends of one set of the A blocks, e.g., in examples in which only one side of the barrier is contacted with the initiator.

FIG. 20B schematically illustrates an example reaction to form barriers that are crosslinked as described with reference to FIG. 2A-2B, 3A-3F, 7A-7B, or 10A-10B. As shown in FIG. 20B, the reaction product of the polymerization of the maleic moieties of an example block copolymer includes poly(maleic acid derivative) formed in a plane at the A-B interface of the block copolymer. Note that although the maleic moieties on the other side of the barrier are not shown, in some examples a similar reaction product may be formed in a plane on the other side of the barrier.

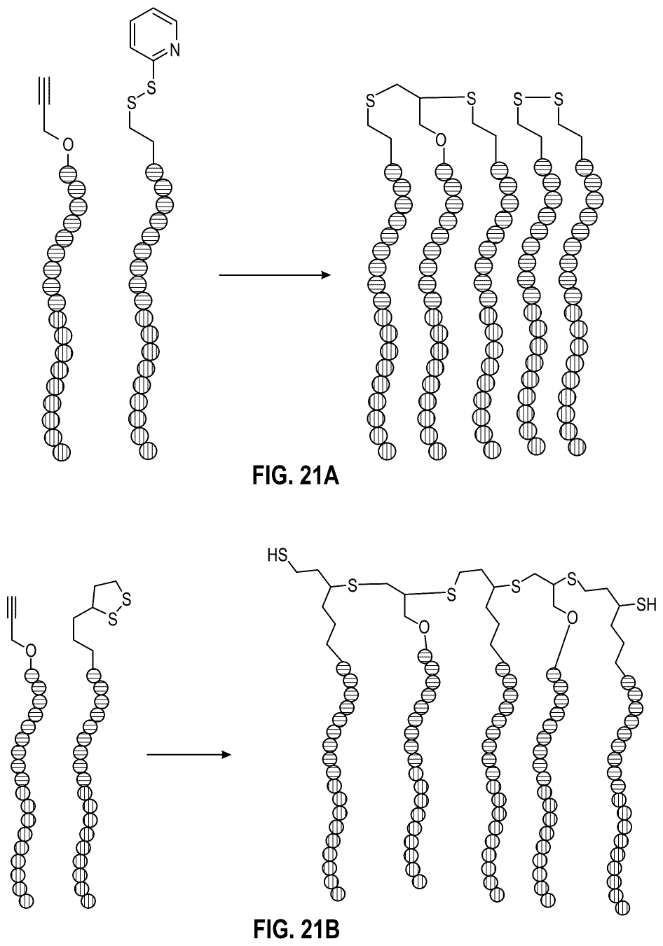

FIG. 21A schematically illustrates an example reaction to form barriers that are crosslinked as described with reference to FIGS. 11A-11B. As shown in FIG. 21A, the reaction product of thiol-yne click coupling between the propargyl moieties of a first block copolymer and the disulfide pyridyl moieties of a second block copolymer includes sulfide bonds formed in a plane at the ends of the A blocks of the block copolymer. Note that the other end of the ABA copolymer is not shown, in some examples similar reaction products may be formed in a plane at the ends of the other A blocks, e.g., in examples in which both sides of the barrier are contacted with the initiator. In other examples, the reaction products substantially may be formed only at the ends of the A blocks on the first side of the barrier, e.g., in examples in which only that side of the barrier is contacted with the initiator.

FIG. 21B schematically illustrates an example reaction to form barriers that are crosslinked as described with reference to FIG. 4A-4D, 5A-5D, 6A-6B, 8A-8B, or 9A-9B. As shown in FIG. 21B, the reaction products of ring-opening and di-thiol formation coupling between the propargyl moieties of a first block copolymer and the lipoamido moieties of a second block copolymer includes sulfide bonds formed in a plane at the ends of the A blocks of the block copolymer. Note that the other end of the ABA copolymer is not shown, in some examples similar reaction products may be formed in a plane at the ends of the other A blocks, e.g., in examples in which both sides of the barrier are contacted with the initiator. In other examples, the reaction products substantially may be formed only at the ends of the A blocks on the first side of the barrier, e.g., in examples in which only that side of the barrier is contacted with the initiator.

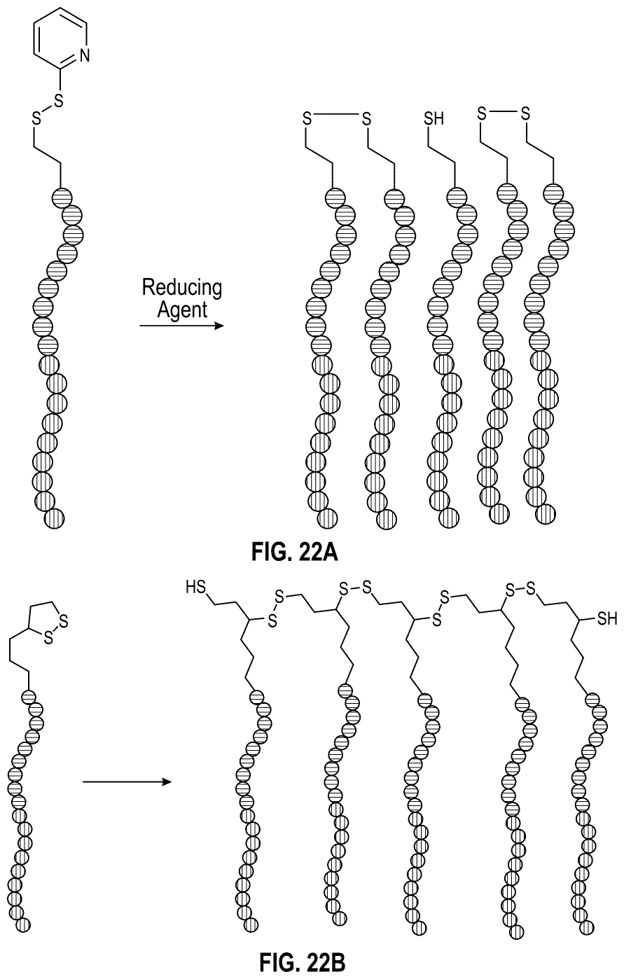

FIG. 22A schematically illustrates an example reaction to form barriers that are crosslinked as described with reference to FIG. 4A-4D, 5A-5D, 6A-6B, 8A-8B, or 9A-9B. As shown in FIG. 22A, the reaction products of the coupling of thiol moieties of a first block copolymer includes a mixture of thiol groups and disulfide bridges formed in a plane at the ends of the A blocks of the block copolymer. Note that although the thiols and disulfide bridges on the other side of the barrier are not shown, in some examples a similar reaction product may be formed in a plane on the other side of the barrier, e.g., in examples in which both sides of the barrier are contacted with a reducing agent. In other examples, the reaction product substantially may be formed only at the ends of one set of the A blocks, e.g., in examples in which only one side of the barrier is contacted with the reducing agent. The reactions in some embodiments may be reversible. For example, a reducing agent may be used to cleave disulfide bridges to obtain free thiols and reverse the cross-linking. Such reversibility may be useful, for example, in applications in which the barrier are shipped cross-linked for stability and then the cross-linking is reversed so the barrier are more fluid during use, e.g., sequencing.

FIG. 22B schematically illustrates an example reaction to form barriers that are crosslinked as described with reference to FIG. 4A-4D, 5A-5D, 6A-6B, 8A-8B, or 9A-9B. As shown in FIG. 22B, the reaction products of the coupling of thiol moieties of a block copolymer includes a mixture of different disulfide bridges formed in a plane at the ends of the A blocks of the block copolymer. Note that although disulfide bridges on the other side of the barrier are not shown, in some examples a similar reaction product may be formed in a plane on the other side of the barrier, e.g., in examples in which both sides of the barrier are contacted with a reducing agent. In other examples, the reaction product substantially may be formed only at the ends of one set of the A blocks, e.g., in examples in which only one side of the barrier is contacted with the reducing agent.

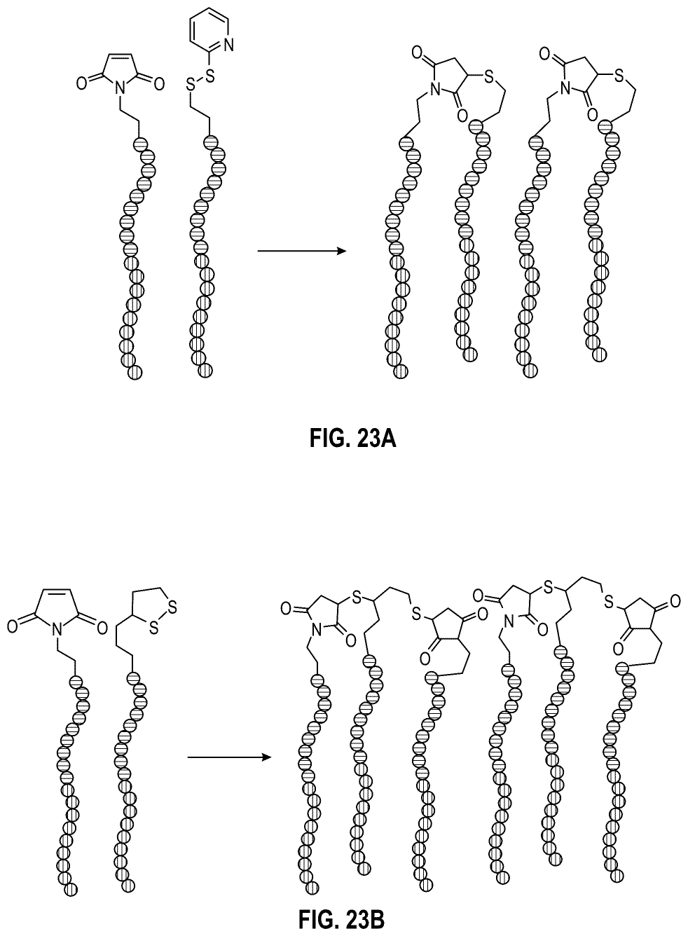

FIG. 23A schematically illustrates an example reaction to form barriers that are crosslinked as described with reference to FIGS. 11A-11B. As shown in FIG. 23A, the reaction products of coupling between the free thiol moieties of a first block copolymer and the maleimide moieties of a second block copolymer includes thiosuccinimide formed in a plane at the ends of the A blocks of the block copolymer. Note that the other end of the ABA copolymer (as one option) is not shown, in some examples similar reaction products may be formed in a plane at the ends of the other A blocks, e.g., in examples in which both sides of the barrier are contacted with a reducing agent. In other examples, the reaction products substantially may be formed only at the ends of the A blocks on the first side of the barrier, e.g., in examples in which only that side of the barrier is contacted with the reducing agent.

FIG. 23B schematically illustrates an example reaction to form barriers that are crosslinked as described with reference to FIGS. 11A-11B. As shown in FIG. 23B, the reaction products of coupling between the free thiol moieties of a first block copolymer and the maleimide moieties of a second block copolymer includes thiosuccinimide formed in a plane at the ends of the A blocks of the block copolymer. Note that the other end of the ABA copolymer (as one option) is not shown, in some examples similar reaction products may be formed in a plane at the ends of the other A blocks, e.g., in examples in which both sides of the barrier are contacted with a reducing agent. In other examples, the reaction products substantially may be formed only at the ends of the A blocks on the first side of the barrier, e.g., in examples in which only that side of the barrier is contacted with the reducing agent.

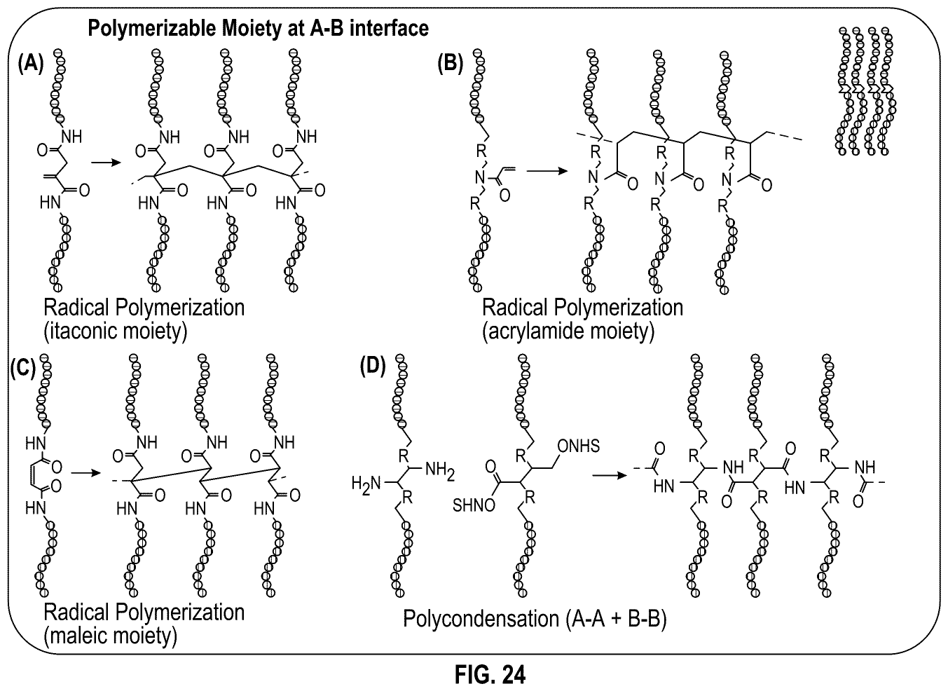

FIGS. 24-27 depict example chemical reactions between different reactive moieties in different locations of the block copolymer, such as described above. For simplicity, only AB block copolymers are depicted, but—where relevant—such reactions also apply for ABA or BAB block copolymers.

More specifically, FIG. 24 illustrates examples in which the A block and B block of a block copolymer are coupled together in a manner that produces/generates/leaves reactive moiet(ies) at the A-B interface, and the moiet(ies) then are reacted to cross-link block copolymer molecules to one another in a manner such as described with reference to FIG. 2A-2B, 3A-3F, 7A-7B, or 10A-10B. In example (A) shown in FIG. 24, the A and B blocks of a block copolymer molecule are coupled together using an itaconic moiety, and the itaconic moieties are polymerized using a radical polymerization process to strengthen at least one layer of the barrier, e.g., in a manner such as described with reference to FIG. 2A-2B, 3A-3F, 7A-7B, or 10A-10B; in some embodiments, a similar process may be used to strengthen a second layer of the barrier, if present. In example (B) shown in FIG. 24, the A and B blocks of a block copolymer molecule are coupled together using an acrylamide moiety, and the acrylamide moieties are polymerized using a radical polymerization process to strengthen at least one layer of the barrier, e.g., in a manner such as described with reference to FIG. 2A-2B, 3A-3F, 7A-7B, or 10A-10B; in some embodiments, a similar process may be used to strengthen a second layer of the barrier, if present. In example (C) shown in FIG. 24, the A and B blocks of a block copolymer molecule are coupled together using a maleic moiety, and the maleic moieties are polymerized using a radical polymerization process to strengthen at least one layer of the barrier, e.g., in a manner such as described with reference to FIG. 2A-2B, 3A-3F, 7A-7B, or 10A-10B; in some embodiments, a similar process may be used to strengthen a second layer of the barrier, if present.

In example (D) shown in FIG. 24, the A and B blocks of a first block copolymer molecule are coupled together using a first moiety, which in the illustrated example includes one or more primary amines (—NH₂), here first and second primary amines; and the A and B blocks of a second block copolymer molecule are coupled together using a second moiety, which in the illustrated example includes one or more NHS esters (—ONHS), here first and second NHS esters. The first moieties (e.g., amine moieties) are reacted with the second moieties (e.g., NHS esters) using a polycondensation process to strengthen at least one layer of the barrier, e.g., using first and second reactive moieties in a manner such as described with reference to FIG. 11A-11B, 12, or 13A-13B, to obtain a structure similar to that described with reference to FIG. 2A-2B, 3A-3F, 7A-7B, or 10A-10B; in some embodiments, a similar process may be used to strengthen a second layer of the barrier, if present. In examples in which each molecule includes two or more amines or two or more NHS esters, two or more of such molecules may be cross-linked with one another. For example, when each molecule includes three or more amines or three or more NHS esters, three or more of such molecules may be cross-linked with one another. The R groups illustrated in examples (A) and (D) of FIG. 24 may include any suitable moiety, such as aliphatic or aromatic or other non-reactive spacer.

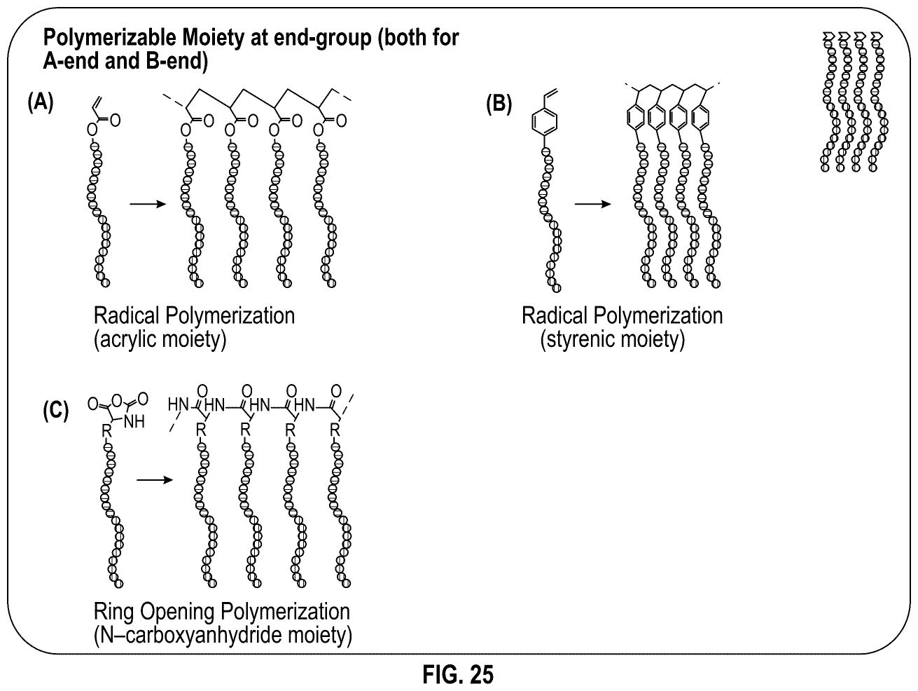

FIG. 25 illustrates examples in which a polymerizable moiety is at an end-group of an A block or at an end-group of a B block, and the moiety then is polymerized to cross-link the molecules of the block copolymer to one another in a manner such as described with reference to FIG. 4A-4D, 5A-5D, 6A-6B, 8A-8B, or 9A-9B. In example (A) shown in FIG. 25, an acrylic moiety is located at the end of an A block or at the end of a B block, and the acrylic moieties are polymerized using a radical polymerization process to strengthen at least one layer of the barrier, e.g., in a manner such as described with reference to FIG. 4A-4D, 5A-5D, 6A-6B, 8A-8B, or 9A-9B; in some embodiments, a similar process may be used to strengthen a second layer of the barrier, if present. In example (B) shown in FIG. 25, a styrenic moiety is located at the end of an A block or at the end of a B block, and the styrenic moieties are polymerized using a radical polymerization process to strengthen at least one layer of the barrier, e.g., in a manner such as described with reference to FIG. 4A-4D, 5A-5D, 6A-6B, 8A-8B, or 9A-9B; in some embodiments, a similar process may be used to strengthen a second layer of the barrier, if present. In example (C) shown in FIG. 25, an N-carboxyanhydride moiety is located at the end of an A block or at the end of a B block, and the N-carboxyanhydride moieties are polymerized using a ring-opening polymerization process to strengthen at least one layer of the barrier, e.g., in a manner such as described with reference to FIG. 4A-4D, 5A-5D, 6A-6B, 8A-8B, or 9A-9B; in some embodiments, a similar process may be used to strengthen a second layer of the barrier, if present. The R groups illustrated in example (C) of FIG. 25 may include any suitable moiety, such as aliphatic or aromatic or other non-reactive spacer.

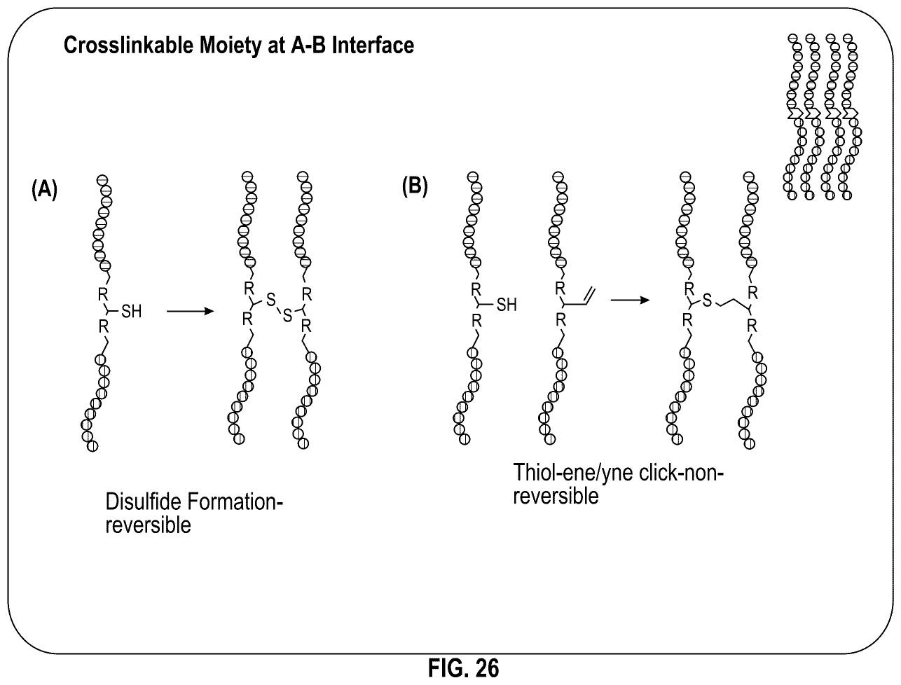

FIG. 26 illustrates additional examples in which the A block and B block of a block copolymer are coupled together using reactive moiet(ies) at the A-B interface, and the moiet(ies) then are reacted to cross-link the molecules of the block copolymer to one another in a manner such as described with reference to FIG. 2A-2B, 3A-3F, 7A-7B, or 10A-10B or 12. In example (A) shown in FIG. 26, the A and B blocks of a block copolymer molecule are coupled together using a moiety including a thiol group (—SH), and the moieties are coupled together using a disulfide formation process to strengthen at least one layer of the barrier, e.g., in a manner such as described with reference to FIG. 2A-2B, 3A-3F, 7A-7B, or 10A-10B; in some embodiments, a similar process may be used to strengthen a second layer of the barrier, if present. In example (A) of FIG. 26, the cross-linking in some embodiments is reversible.

In example (B) shown in FIG. 26, the A and B blocks of a first block copolymer molecule are coupled together using a first moiety, which in the illustrated example includes one or more thiol groups (—SH); and the A and B blocks of a second block copolymer molecule are coupled together using a second moiety, which in the illustrated example includes one or more alkynes or alkenes. The first moieties (e.g., thiol moieties) are reacted with the second moieties (e.g., alkynes or alkenes) for example, using a thiol-ene/yne click chemistry process (which is not reversible), to strengthen at least one layer of the barrier, e.g., using first and second reactive moieties in a manner such as described with reference to FIG. 11A-11C, 12, or 13A-13B, to obtain a structure similar to that described with reference to FIG. 2A-2B, 3A-3F, 7A-7B, 10A-10B, or 12; in some embodiments, a similar process may be used to strengthen a second layer of the barrier, if present. In examples in which each molecule includes two or more thiols or two or more alkynes or alkenes, two or more of such molecules may be cross-linked with one another. For example, when each molecule includes three or more thiols or three or more alkenes, three or more of such molecules may be cross-linked with one another. The R groups illustrated in examples (A) and (B) of FIG. 26 may include any suitable moiety, such as aliphatic or aromatic or other non-reactive spacer.

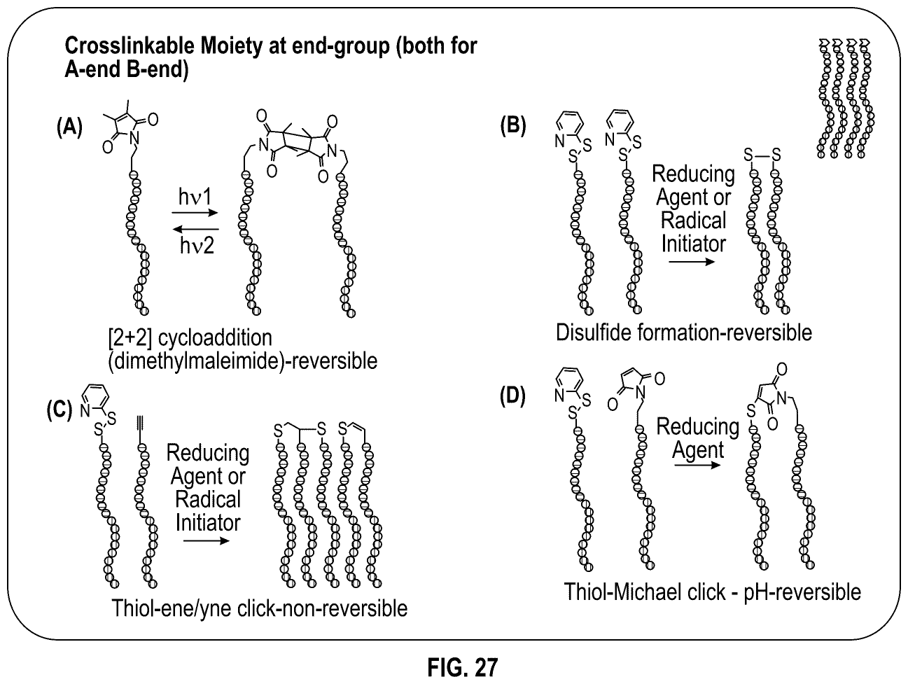

FIG. 27 illustrates additional examples in which reactive moiet(ies) are at an end-group of an A block or at an end-group of a B block, and the moiet(ies) then are reacted to cross-link the molecules of the block copolymer to one another in a manner such as described with reference to FIG. 4A-4D, 5A-5D, 6A-6B, 8A-8B, 9A-9B, 11A-11C, or 13A-13B. In example (A) shown in FIG. 27, dimethylmaleimide is located at the end of an A block or at the end of a B block, and the dimethylmaleimide moieties are reacted in a [2+2] cycloaddition process to strengthen at least one layer of the barrier, e.g., in a manner such as described with reference to FIG. 4A-4D, 5A-5D, 6A-6B, 8A-8B, or 9A-9B; in some embodiments, a similar process may be used to strengthen a second layer of the barrier, if present. In example (A) of FIG. 27, the cross-linking in some embodiments is reversible. In example (B) shown in FIG. 27, a disulfide pyridyl moiety is located at the end of an A block or at the end of a B block, and the disulfide pyridyl moieties are polymerized using a disulfide formation process (which may use a reducing agent or radical initiator) to strengthen at least one layer of the barrier, e.g., in a manner such as described with reference to FIG. 4A-4D, 5A-5D, 6A-6B, 8A-8B, or 9A-9B; in some embodiments, a similar process may be used to strengthen a second layer of the barrier, if present. In example (B) of FIG. 27, the cross-linking in some embodiments is reversible.

In example (C) shown in FIG. 27, a first block copolymer molecule includes a first moiety (e.g., disulfide pyridyl in the illustrated example) and a second block copolymer molecule includes a second moiety (e.g., alkene or alkyne in the illustrated example). The first moiety (e.g., disulfide pyridyl) is reacted with one or more of the second moieties (e.g., alkyne(s) or alkene(s)), for example using a thiol-ene/yne click chemistry process (which is not reversible), to strengthen at least one layer of the barrier, e.g., using first and second reactive moieties in a manner such as described with reference to FIG. 11A-11C, 12, or 13A-13B, to obtain a structure similar to that described with reference to FIG. 4A-4D, 5A-5D, 6A-6B, 8A-8B, 9A-9B, 11A-11C, or 13A-13B; in some embodiments, a similar process may be used to strengthen a second layer of the barrier, if present. In the illustrated example, an alkyne may react with up to two thiols. The first reaction between the yne and thiol moieties consumes the triple bond and generates a double bond, which in turn can react with another thiol.

In example (D) shown in FIG. 27, a first block copolymer molecule includes a first moiety (e.g., disulfide pyridyl in the illustrated example) and a second block copolymer molecule includes a second moiety (e.g., maleimide in the illustrated example). The first moiety (e.g., disulfide pyridyl) is reacted with the second moiety (e.g., maleimide), for example using a thiol-Michael click chemistry process (which is pH reversible), to strengthen at least one layer of the barrier, e.g., using first and second reactive moieties in a manner such as described with reference to FIG. 11A-11C, 12, or 13A-13B, to obtain a structure similar to that described with reference to FIG. 4A-4D, 5A-5D, 6A-6B, 8A-8B, 9A-9B, 11A-11C, or 13A-13B; in some embodiments, a similar process may be used to strengthen a second layer of the barrier, if present.

Monomers 321, 321′ described with reference to FIGS. 3A-3F, and elsewhere herein, may have any suitable structure and may include any suitable reactive moiety that may be used to polymerize the monomers. For example, monomers 321, 321′ may have the structure:

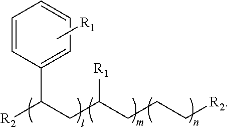

In some examples, monomers may contain a combination of 0, 1 or more units of groups l, m or n. In some examples, R₁ is selected from the group consisting of:

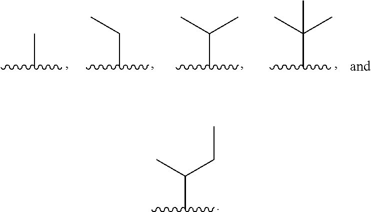

In some examples, the reactive moieties R₂ of the monomers 321, 321′ may be selected from the group consisting of:

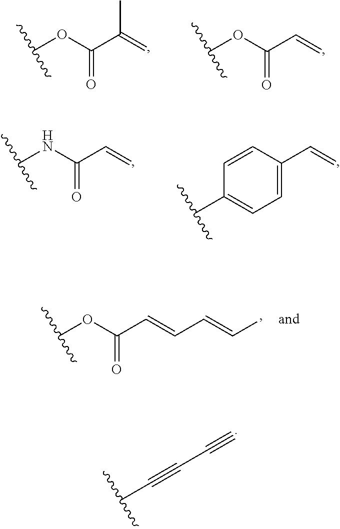

In one specific, nonlimiting example, the reactive moiety may include methyl methacrylate, and the monomer 321 may have the structure:

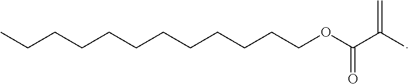

### Lauryl Methacrylate.

Additionally, or alternatively, in one specific, nonlimiting example, the reactive moiety may include methyl methacrylate, and the monomer 321′ may have the following structure:

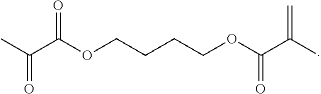

### 1,4-Butanediol dimethacrylate

In a manner such as noted with reference to FIGS. 3A-3F, the polymerization reaction(s) between moieties 311 and one another, between moieties 350 and one another, and/or between moieties 311 and moieties 350 in some embodiments may be initiated using an initiator 390. Nonlimiting examples of suitable initiators include a photoinitiator, a redox system, or photons (such as ultraviolet (UV) light). Illustratively, the photoinitiator is UV activated and is selected from the group consisting of: 2,2-dimethoxy-2-phenylacetophenone, 2,2′-azobis(2-methylpropionamidine) dihydrochloride, 2-hydroxy-4′-(2-hydroxyethoxy)-2-methylpropiophenone, and lithium phenyl-2,4,6,-trimethylbenzoylphosphinate, structures for which are shown below:
2,2-dimethoxy-2-phenylacetophenone (DMAP):

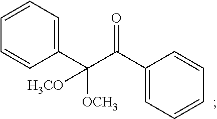 [SMILES: COC(OC)(C(=O)c1ccccc1)c1ccccc1]

2,2′-azobis(2-methylpropionamidine) dihydrochloride (V50):

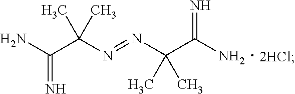

2-hydroxy-4′-(2-hydroxyethoxy)-2-methylpropiophenone (Irgacure 2959):

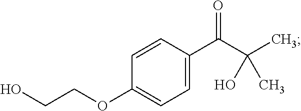 [SMILES: CC(C)(O)C(=O)c1ccc(OCCO)cc1]

and
lithium phenyl-2,4,6,-trimethylbenzoylphosphinate:

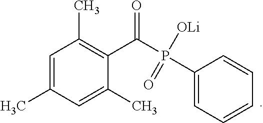

In examples described herein which UV light is used, the barrier may be located within a structure which is at least partially transparent to the UV light, so as to facilitate cross-linking and/or reversing cross-linking. For example, the barrier may be located within a flowcell the lid of which may be at least partially transparent to the UV light used for cross-linking and/or reversing cross-linking, such that a sufficient amount of the UV light reaches the barrier to sufficiently conduct the reaction.

In some examples, the redox system includes potassium persulfate and N,N,N′,N′-tetramethylethylenediamine, the structures of which are shown below:

potassium persulfate (KPS):

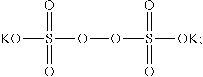

and
N,N,N′,N′-tetramethylethylenediamine (TEMED):

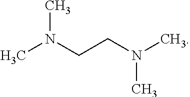

Ammonium persulfate and TEMED alternatively may be used as the redox system.

As noted elsewhere herein, if monomers 321, 321′ are included, their reaction may be initiated using an initiator 390 which is the same as, or different than, reaction of moiety 311 of amphiphilic molecules 221. The initiator(s) may be provided in any suitable location to initiate polymerization of the monomers and/or of any reactive groups of the amphiphilic molecules. FIG. 14 schematically illustrates an alternative manner in which the operations described with reference to FIG. 3C or 3F may be performed. In the nonlimiting example illustrated in FIG. 14, hydrophobic initiator 390 is provided within hydrophobic liquid 303, e.g., during initial formation of barrier 300 such as described with reference to FIG. 3A. The hydrophobic initiator 390 may be provided in any suitable weight percent (wt %) of liquid 303 to cause monomers 321, 321′ to polymerize and/or to cause moieties 311 to react. An example hydrophobic radical initiator that suitably may be used to polymerize acrylates to form polyacrylate is 2,2-dimethoxy-2-phenylacetophenone:

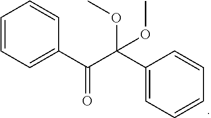 [SMILES: COC(OC)(C(=O)c1ccccc1)c1ccccc1]

Another example hydrophobic radical initiator that suitably may be used to polymerize acrylates to form polyacrylate is benzoyl peroxide:

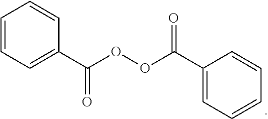 [SMILES: O=C(OOC(=O)c1ccccc1)c1ccccc1]

Alternatively, the initiator may be hydrophilic and used a manner such as described with reference to FIGS. 3A-3F. More specifically, in the nonlimiting example illustrated in FIGS. 3A-3C and 3E-3F, hydrophilic initiator 390 is provided within aqueous liquid(s) 313 respectively in contact with the first and second sides of the barrier, e.g., during or following thinning of the barrier such as described with reference to FIG. 3B. The hydrophilic initiator may be provided in any suitable weight percent (wt %) of the aqueous liquid(s) to cause monomers 321, 321′ to polymerize and/or to cause moieties 311 to react. Example hydrophilic initiators that suitably may be used to polymerize acrylates to form polyacrylate are selected from the group consisting of:

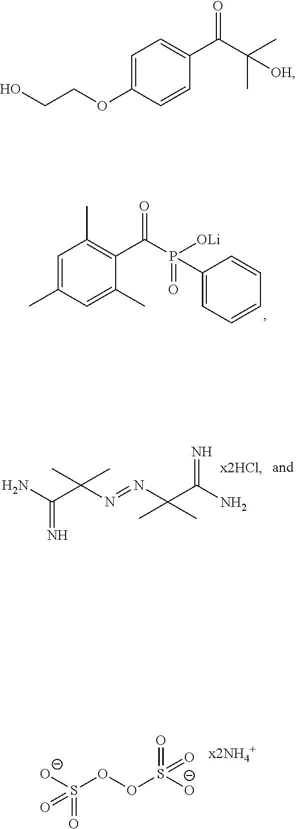

Irgacure 2959 (2-Hydroxy-4′-(2-hydroxyethoxy)-2-methylpropiophenone), LAP (Lithium phenyl-2,4,6-trimethylbenzoylphosphinate), V-50 (2,2′-Azobis(2-methylpropionamidine) dihydrochloride, and APS (ammonium persulfate).

FIG. 15 schematically illustrates an alternative manner in which the operations described with reference to FIG. 3C or 3F may be performed. In the nonlimiting example illustrated in FIG. 15, nanopore 110 may be coupled to initiator(s) 390. For example, the initiator(s) may be coupled directly to suitable amino acid moieties of the nanopore 110 or indirectly via linkers (not specifically labeled). The nanopore thus modified may be inserted into the barrier at any suitable time, e.g., during operations such as described with reference to FIG. 3D. The initiator(s) 390 may be provided in any suitable number and distribution to cause monomers 321, 321′ to polymerize and/or to cause moieties 311 to react. Example initiators are described elsewhere herein that suitably may be linked to a nanopore and used to initiate suitable reactions.

It will be appreciated that initiators 390 such as described herein, e.g., with reference to FIGS. 3A-3F, 14, and 15 in some embodiments may be used in conjunction with light, such as UV light of a suitable wavelength to initiate polymerization of monomers 321, 321′ and/or reaction of moieties 311. Additionally, or alternatively, initiators 390 in some embodiments may be activated using temperature and/or basic conditions.

FIG. 16 schematically illustrates an example manner in which a barrier may be covalently coupled to a nanopore during operations such as described with reference to FIGS. 3A-3F. In the nonlimiting example illustrated in FIG. 16, nanopore 110 includes reactive moieties 1611 that can react with moieties 350 of monomers 321, 321′ and/or that can react with moieties 311 of the amphiphilic molecules 221. For example, reactive moieties 1611 may be of the same type as reactive moieties 350 and/or may be of the same type as moieties 311. Accordingly, when monomers 321, 321′ and/or amphiphilic molecules 221 react, reactive moieties 350 and/or moieties 311 may become coupled to reactive moieties 1611 in such a way as to covalently attach polymer 203 and/or amphiphilic molecules 221 to support 200. Reactive moieties 1611 may be suitably coupled to nanopore 110, e.g., via linkers which are attached to residues of the nanopore.

FIG. 17 schematically illustrates an example manner in which a barrier may be covalently coupled to a barrier support during operations such as described with reference to FIGS. 3A-3F. In the nonlimiting example illustrated in FIG. 17, support 200 includes reactive moieties 1711 that can react with moieties 350 of monomers 321, 321′ and/or that can react with moieties 311 of the amphiphilic molecules 221. For example, reactive moieties 1711 may be of the same type as reactive moieties 350 and/or may be of the same type as moieties 311. Accordingly, when monomers 321, 321′ and/or amphiphilic molecules 221 react, reactive moieties 350 and/or moieties 311 may become coupled to reactive moieties 1711 in such a way as to covalently attach polymer 203 and/or amphiphilic molecules 221 to support 200. Reactive moieties 1711 may be suitably coupled to support 200, e.g., via linkers, such as silane groups which support 200 is chemically modified so as to include.

While FIGS. 3A-10B may illustrate cross-linking of amphiphilic molecules using polymerization, it will be appreciated that other types of crosslinking reactions, such as coupling reactions, suitably may be used to crosslink the amphiphilic molecules. For example, FIGS. 11A-11B schematically illustrate example operations for forming another alternative barrier including a nanopore and crosslinked amphiphilic molecules. FIG. 11A illustrates suspended barrier 1100. As illustrated in FIG. 11A, barrier 1100 may be configured, in some regards, similarly as barrier 101 described with reference to FIGS. 1 and 2A-2B, e.g., may include layer 1101 including a first plurality of amphiphilic molecules 221 and layer 1102 including a second plurality of amphiphilic molecules 221. Some of the amphiphilic molecules 221 may include reactive moieties 1111 while other of the amphiphilic molecules 221 may include reactive moieties 1112 which are different than reactive moieties 1111. In examples such as illustrated in FIG. 11A, the amphiphilic molecules 1121, 1122 include molecules of an AB diblock copolymer, of which the hydrophilic “A” sections 1132 of molecules 121 may include either reactive moiety 1111 or reactive moiety 1112, e.g., coupled to the terminal hydrophilic monomer. In other examples, just one type of reactive moiety is used. In a manner similar to that described with reference to FIGS. 3B-3F, cross-linking reactions of reactive moieties 1111 and 1112 may be used so as to only partially cross-link amphiphilic molecules 221 to one another via bonds 280, then nanopore 110 may be inserted into the barrier, and then cross-linking of reactive moieties 1111 and 1112 may be used to further crosslink amphiphilic molecules 221 to one another, e.g., forming additional bonds 280 illustrated in FIG. 11B. Although not specifically illustrated in FIGS. 11A-11B, it should be understood that the barrier in some embodiments also may include monomers 321, 321′ which may be reacted in a manner such as described with reference to FIGS. 3A-3F to form polymer 203 and/or to form bonds 250 with molecules 221.

FIG. 11B illustrates the products of polymerization reactions between the amphiphilic molecules, in which bonds 1180 are formed between reactive moieties 1111 and 1112 (the fill of which is changed from crosshatched to white to indicate that such moieties have reacted and are no longer available for reaction). Although FIG. 11B may suggest that each reactive moiety 1111 is cross-linked to two of moieties 1112 via respective bonds 1180 and that each reactive moiety 1112 is cross-linked to two of moieties 1111 via respective bonds 1180, it will be appreciated that each reactive moiety may form bonds with any suitable number of other such reactive moieties, e.g., one, two, three, or more than three other such reactive moieties, and that bonds 1180 can be of different types than one another, e.g., may include different moieties than one another. The relative proportion of such products may be controlled in a manner such as described elsewhere herein, e.g., through the type of reactive moieties used, the type of initiator used, and the reaction conditions, so as to control the amount of cross-linking provided using reactions between the reactive moieties 1111 and 1112. Additionally, in some examples, the amount of cross-linking may be controlled by mixing the amphiphilic molecules respectively including reactive moieties 1111, 1112 in suitable proportion with other amphiphilic molecules that do not include reactive moieties 1111 and 1112, or that include different reactive moieties, and/or that have a different architecture (e.g., AB can be mixed with ABA and/or BAB; ABA can be mixed with AB and/or BAB; and/or BAB can be mixed with AB and/or ABA). For example, the ratio between the different types of amphiphilic molecules may be selected so as to determine the extent of cross-linking. Illustratively, in the case where two different amphiphilic molecules are used, the ratio may be selected so as to provide substantially full cross-linking between the molecules whereas a lower ratio may leave some molecules unreacted and thus only partially cross-linked.

In a similar manner as described with reference to FIGS. 3A-3F, 4A-4D, 5A-5D, 6A-6B, 7A-7B, 8A-8B, 9A-9B, and 10A-10B, the type(s) of amphiphilic molecules used, and the locations of the reactive moieties 1111, 1112 within such molecules, suitably may be varied. For example, moieties 1111 and 1112 instead may be located at the A-B interface of the molecules of FIG. 11A-11C, or instead may be located at the B block of the molecules of FIGS. 11A-11C. Or, for example, moieties 1111 and 1112 instead may be provided within ABA triblock copolymers, e.g., at the A block, at the A-B interface, or at the B block. Or, for example, moieties 1111 and 1112 instead may be provided within BAB triblock copolymers, e.g., at the A block, at the A-B interface, or at the B block. FIG. 12 schematically illustrates an example operation for forming another alternative barrier including a nanopore and crosslinked amphiphilic molecules, in which moieties 1111 and 1112 are provided at the A-B interface of ABA triblock copolymers. FIG. 13A illustrates an example operation for forming another alternative barrier including crosslinked amphiphilic molecules, in which moieties 1111 and 1112 are provided at the B block of BAB triblock copolymers. The barriers are illustrated in FIGS. 12 and 13A prior to crosslinking, and suitably may be crosslinked in a manner to form bonds 280 such as provided herein, e.g., with reference to FIGS. 11A-11C.

A variety of reaction schemes may be used in coupling reactions such as described with reference to FIGS. 11A-13B. For example, the coupling reaction may include a thiol-ene click reaction, a thiol-yne click reaction, a strain-promoted alkyne-azide cycloaddition (e.g., an azide with DBCO or BCN), an amide coupling (primary amide with N-hydroxysuccinimide (NHS) or pentafluorophenyl (PFP)-activated esters), a thiol/aza-Michael reaction (thiol/primary amine with maleimide, maleic, fumaric, acrylic, or acrylamide), a [2+2] photocycloaddition (e.g., dimethylmaleimide, enones, or coumarin), a protein-ligand interaction (e.g., biotin-avidin or biotin-streptavidin), condensation (e.g., amine with an NHS ester), or host-guest chemistry (e.g., cyclodextrin-adamantane). Such reactions may be irreversible. Alternatively, reversible reactions may be used such as a disulfide formation, an imine formation, [2+2] cycloaddition, thiol-Michael click reaction, or an enamine formation (e.g., aldehyde/ketone). Nonlimiting examples of reactive moieties 1111, 1112 may include a disulfide pyridyl moiety, a lipoamido moiety, a propargyl moiety, an azide moiety, a DBCO moiety, a BCN moiety, an amine moiety, an activated carboxylic moiety, a dimethylmaleimide moiety, a propargyl moiety, an NHS ester, or a maleimide moiety. In some embodiments, the coupling reaction may be initiated using an initiator, such as a free-radical initiator, a redox system, a reducing agent, or photons. Nonlimiting examples of free-radical initiators include 2-hydroxy-4′-(2-hydroxyethoxy)-2-methylpropiophenone and 2,2′-azobis(2-methylpropionamidine) dihydrochloride, structures of which are provided above. A nonlimiting example of a redox system is potassium persulfate or ammonium persulfate and N,N,N′,N′-tetramethylethylenediamine, structures of which are provided above. Nonlimiting examples of reducing agents include tris(2-carboxyethyl) phosphine, dithiothreitol, sodium ascorbate, and a phosphine.

FIG. 11C schematically illustrates example diblock copolymer molecules that may be used in operations such as described with reference to FIGS. 11A-11B; and FIG. 13B schematically illustrates example triblock copolymer molecules that may be used in operations such as described with reference to FIG. 13A. From left to right in FIGS. 11C and 13B, the groups include acrylamide (used for polymerization reaction), methacrylamide (used for polymerization reaction), penta-fluoro benzyl methacrylate (used for polymerization reaction), and thiol (used for coupling reaction). Other nonlimiting examples are described with reference to FIGS. 24-27. Still other nonlimiting examples are described elsewhere herein.

FIGS. 18A-18C schematically illustrate further details of barriers using block copolymers which may be included in the nanopore composition and device of FIG. 1 and used in respective operations described with reference to FIGS. 3A-17. It will be appreciated that such barriers suitably may be adapted for use in any other composition or device, and are not limited to use with nanopores. The amphiphilic molecules of the barriers described with reference to FIGS. 18A-18C may include reactive moieties 311, 1111, or 1112 such as described elsewhere herein.

Referring now to FIG. 18B, barrier 1801 uses a diblock “AB” copolymer. Barrier 1801 includes first layer 1807 which may contact fluid 120 and second layer 1808 which may contact fluid 120′ in a manner similar to that described with reference to FIG. 1. First layer 1807 includes a first plurality of molecules 1802 of a diblock AB copolymer, and second layer 1808 includes a second plurality of the molecules 1802 of the diblock AB copolymer. As illustrated in FIG. 18B, each molecule 1802 of the diblock copolymer includes a hydrophobic block, denoted “B” and being approximately of length “B,” coupled to a hydrophilic block, denoted “A” and being approximately of length “A”. The hydrophilic A blocks of the first plurality of molecules 1802 (the molecules forming layer 1807) form a first outer surface of the barrier 1801, e.g., contact fluid 120. The hydrophilic A blocks of the second plurality of molecules 1802 (the molecules forming layer 1808) form a second outer surface of the barrier 1802, e.g., contact fluid 120′. The respective ends of the hydrophobic B blocks of the first and second pluralities of molecules contact one another within the barrier 1801 in a manner such as illustrated in FIG. 18B. As illustrated, substantially all of the molecules 1802 within layer 1807 may extend substantially linearly and in the same orientation as one another, and similarly substantially all of the molecules 1802 within layer 1808 may extend substantially linearly and in the same orientation as one another (which is opposite that of the orientation the molecules within layer 1807). Accordingly, first and second layers 1807, 1808 each may have a thickness of approximately A+B, and barrier 1801 may have a thickness of approximately 2A+2B. In some examples, length A is about 2 repeating units (RU) to about 100 RU, or about 1 repeating unit (RU) to about 50 RU, e.g., about 5 RU to about 40 RU, or about 10 RU to about 30 RU, or about 10 RU to about 20 RU, or about 20 RU to about 40 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU. In some embodiments, barrier 1801 described with reference to FIG. 18B may be suspended across an aperture in a manner such as described with reference to FIGS. 2A-2B.

Referring now to FIG. 18C, barrier 1811 uses a triblock “BAB” copolymer. Barrier 1811 includes first layer 1817 which may contact fluid 120 and second layer 1818 which may contact fluid 120′ in a manner similar to that described with reference to FIG. 1. First layer 1817 includes a first plurality of molecules 1812 of a triblock copolymer, and second layer 1818 includes a second plurality of the molecules 1812 of the triblock copolymer. As illustrated in FIG. 18C, each molecule 1812 of the triblock copolymer includes first and second hydrophobic blocks, each denoted “B” and being approximately of length “B,” and a hydrophilic block disposed between the first and second hydrophobic blocks, denoted “A” and being approximately of length “A”. The hydrophilic A blocks of the first plurality of molecules 1812 (the molecules forming layer 1817) form a first outer surface of the barrier 1811, e.g., contact fluid 120. The hydrophilic A blocks of the second plurality of molecules 1812 (the molecules forming layer 1818) form a second outer surface of the barrier 1811, e.g., contact fluid 120′. The respective ends of the hydrophobic B blocks of the first and second pluralities of molecules contact one another within the barrier 1811 in a manner such as illustrated in FIG. 18C. As illustrated, substantially all of the molecules 1812 within layer 1817 may extend in the same orientation as one another, and may be folded at the A block so that the A block can contact the fluid while the B blocks are interior to the barrier 1811. Similarly, substantially all of the molecules 1812 within layer 1818 may extend in the same orientation as one another (which is opposite that of the orientation the molecules within layer 1817), and may be folded at their A blocks so that the A blocks contact the fluid while the B blocks are interior to the barrier 1811. Accordingly, first and second layers 1817, 1818 each may have a thickness of approximately A/2+B, and barrier 1811 may have a thickness of approximately A+2B. In some examples, length A is about 2 RU to about 100 RU, e.g., about 10 RU to about 80 RU, or about 20 RU to about 50 RU, or about 50 RU to about 80 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU. In some embodiments, barrier 1811 described with reference to FIG. 18B may be suspended across an aperture in a manner such as described with reference to FIGS. 2A-2B.

Referring now to FIG. 18A, barrier 1821 uses a triblock “ABA” copolymer. Barrier 1821 includes layer 1829 which may contact both fluids 120 and 120′. Layer 1829 includes a plurality of molecules 1822 of a triblock ABA copolymer. As illustrated in FIG. 18A, each molecule 1822 of the triblock copolymer includes first and second hydrophilic blocks, each denoted “A” and being approximately of length “A,” and a hydrophobic block disposed between the first and second hydrophilic blocks, denoted “B” and being approximately of length “B”. The hydrophilic A blocks at first ends of molecules 1822 (the molecules forming layer 1829) form a first outer surface of the barrier 1821, e.g., contact fluid 120. The hydrophilic A blocks at second ends of molecules 1822 form a second outer surface of the barrier 1821, e.g., contact fluid 120′. The hydrophobic B blocks of the molecules 1822 are within the barrier 1811 in a manner such as illustrated in FIG. 18C. As illustrated, the majority of molecules 1822 within layer 1829 may extend substantially linearly and in the same orientation as one another. In some embodiments, as illustrated in FIG. 18A, some of the molecules 1822′ may be folded at their B blocks, such that both of the hydrophilic A blocks of such molecules may contact the same fluid as one another. Accordingly, the example shown in FIG. 18A may be considered to be partially a single layer, and partially a bilayer. In other examples (not specifically illustrated), layer 1829 may be entirely a single-layer or may be entirely a bilayer, e.g., as also described elsewhere herein. Regardless of whether the barrier includes molecules 1822 which extend substantially linearly and/or molecules 1822′ which are folded, as illustrated in FIG. 18A, layer 1829 may have a thickness of approximately 2A+B. In some examples, length A is about 1 RU to about 100 RU, e.g., about 2 RU to about 100 RU, or about 10 RU to about 80 RU, or about 20 RU to about 50 RU, or about 50 RU to about 80 RU. Additionally, or alternatively, in some examples, length B is about 2 RU to about 100 RU, or about 5 RU to about 100 RU, e.g., about 10 RU to about 80 RU, or about 20 RU to about 50 RU, or about 50 RU to about 80 RU. It will be appreciated that any end groups that are coupled to the hydrophilic or hydrophobic blocks contribute to the overall thickness of the barrier. In some embodiments, barrier 1821 described with reference to FIG. 18A may be suspended across an aperture in a manner such as described with reference to FIGS. 2A-2B.

It will be appreciated that the layers of the various barriers provided herein may be configured so as to have any suitable dimensions. Illustratively, to form barriers of similar dimension as one another:

A-B-A triblock copolymer (FIG. 18A) may have 2 hydrophilic blocks, each of length A (each A block is of M_{w}=x) and 1 hydrophobic block of length B (M_{w}=y); when self-assembled, those A-B-A triblock copolymers would form barriers with a top hydrophilic layer of length A, a core hydrophobic layer of length B, and a bottom hydrophilic layer of length A.

A-B diblock copolymer (FIG. 18B) may have 1 hydrophilic block of length A (M_{w}=X), and 1 hydrophobic block of length B (M_{w}=y/2); when self-assembled, those A-B diblock copolymers would form barriers with a top hydrophilic layer of length A, a core hydrophobic layer of length 2B, and a bottom hydrophilic layer of length A.

B-A-B triblock copolymer (FIG. 18C) may have 1 hydrophilic block of length A (M_{w}=X), and 2 hydrophobic blocks, each of length of B (each B block is of M_{w}=y/2); when self-assembled, those B-A-B triblock copolymers would form barriers with a top hydrophilic layer of length A/2, a core hydrophobic layer of length 2B, and a bottom hydrophilic layer of length A/2. Additionally, or alternatively, the polymer packing into the layer(s) of the barrier may affect the hydrophilic ratio for each of the barriers, where hydrophilic ratio may be defined as the ratio between molecular mass of the hydrophilic block and the total molecular weight (MW or M_{w}) of the block copolymer (BCP) (hydrophilic ratio=M_{w} hydrophilic block/M_{w} BCP). For example:

A-B-A triblock copolymer (FIG. 18A), hydrophilic ratio=2x/(2x+y);
A-B diblock copolymer (FIG. 18B), hydrophilic ratio=x/(x+y/2); and
B-A-B triblock copolymer (FIG. 18C), hydrophilic ratio=x/(x+y).

The present diblock and triblock copolymers may include any suitable combination of hydrophobic and hydrophilic blocks. In some examples, the hydrophilic A block may include a polymer selected from the group consisting of: N-vinyl pyrrolidone, polyacrylamide, zwitterionic polymer, hydrophilic polypeptide, nitrogen containing units, and poly(ethylene oxide) (PEO). Illustratively, the polyacrylamide may be selected from the group consisting of: poly(N-isopropyl acrylamide) (PNIPAM), and charged polyacrylamide, and phosphoric acid functionalized polyacrylamide. Nonlimiting examples of zwitterionic monomers that may be polymerized to form zwitterionic polymers include:

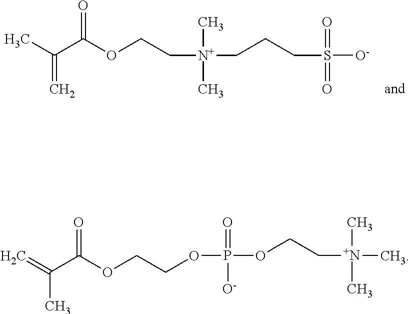 [SMILES: C=C(C)C(=O)OCC[N+](C)(C)CCCS(=O)(=O)[O-] ; C=C(C)C(=O)OCCOP(=O)([O-])OCC[N+](C)(C)C]

Nonlimiting examples of hydrophilic polypeptides include:

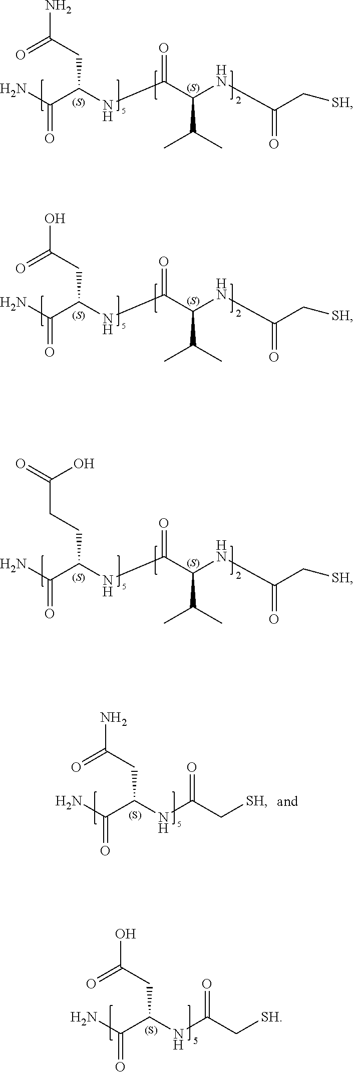

A nonlimiting example of a charged polyacrylamide is

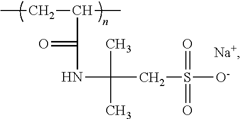

where n is between about 1 and about 100.
Nonlimiting examples of nitrogen containing units include:

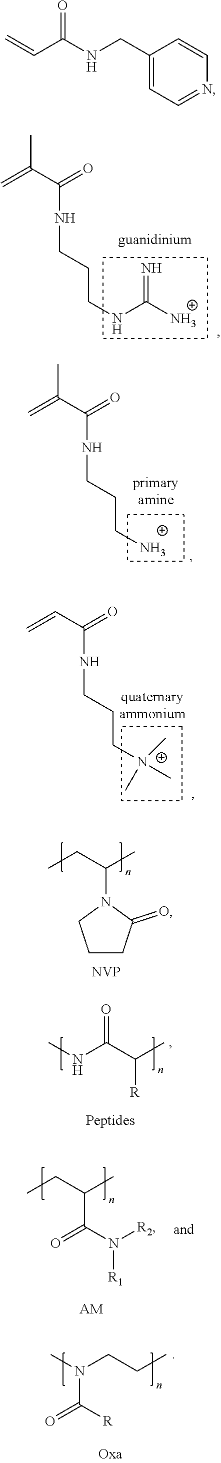

In some examples, the hydrophobic B block may include a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). Nonlimiting examples of hydrogenated polydienes include saturated polybutadiene (PBu), saturated polyisoprene (PI), saturated poly(myrcene),

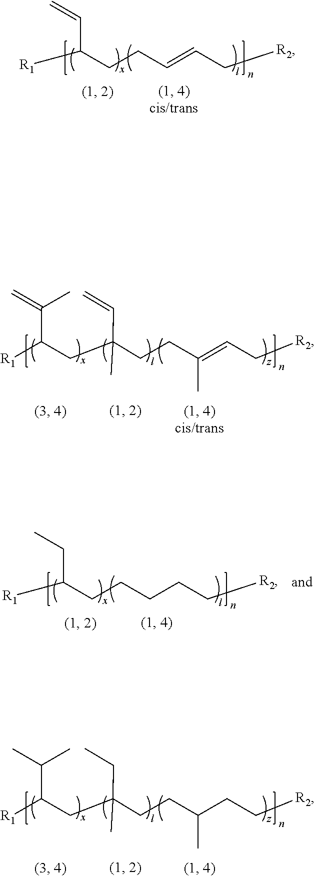

where n is between about 2 and about 100, x is between about 2 and about 100, y is between about 2 and about 100, z is between about 2 and about 100, R₁ is a functional group selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, an orthogonal functionality, and a hydrogen, and R₂ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. In other nonlimiting examples of hydrogenated polydienes, R₁ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. A nonlimiting example of fluorinated polyethylene is

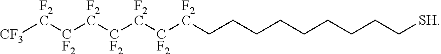 [SMILES: FC(F)(F)C(F)(F)C(F)(F)C(F)(F)C(F)(F)C(F)(F)C(F)(F)C(F)(F)CCCCCCCCCS]

Nonlimiting examples of hydrophobic polypeptides include (0<x<1):

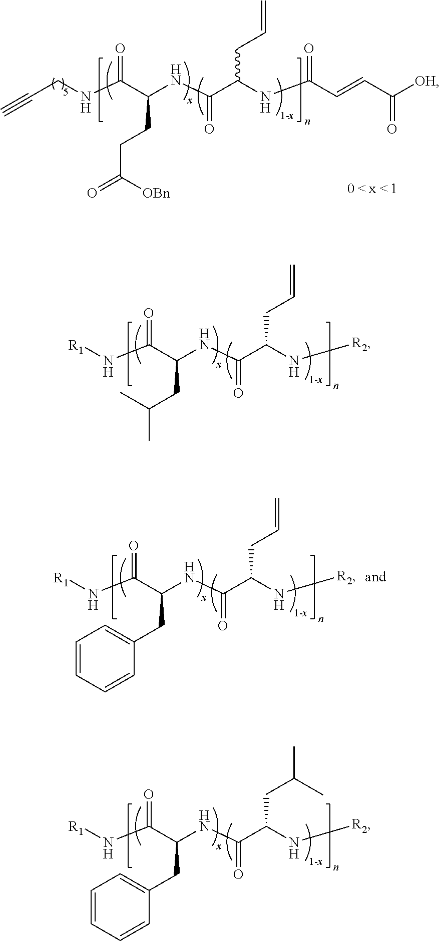

where n is between about 2 and about 100.

In one nonlimiting example, an AB diblock copolymer includes PDMS-b-PEO, where “-b-” denotes that the polymer is a block copolymer. In another nonlimiting example, an AB diblock copolymer includes PBd-b-PEO. In another nonlimiting example, an AB diblock copolymer includes PIB-b-PEO. In another nonlimiting example, a BAB triblock copolymer includes PDMS-b-PEO-b-PDMS. In another nonlimiting example, a BAB triblock copolymer includes PBd-b-PEO-b-PBd. In another nonlimiting example, a BAB triblock copolymer includes PIB-b-PEO-b-PIB. In another nonlimiting example, an ABA triblock copolymer includes PEO-b-PBd-b-PEO. In another nonlimiting example, and ABA triblock copolymer includes PEO-b-PDMS-b-PEO. In another nonlimiting example, an ABA triblock copolymer includes PEO-b-PIB-b-PEO. It will be appreciated that any suitable hydrophilic block(s) may be used with any suitable hydrophobic block(s). Additionally, in examples including two hydrophilic blocks, those blocks may be but need not necessarily include the same polymers as one another. Similarly, in examples including two hydrophobic blocks, those blocks may be but need not necessarily include the same polymers as one another.

The respective molecular weights, glass transition temperatures, and chemical structures of the hydrophobic and hydrophilic blocks suitably may be selected so as to provide the barrier with appropriate stability for use and ability to insert a nanopore. For example, the respective molecular weights of the hydrophobic and hydrophilic blocks may affect how thick each of the blocks (and thus layers of the barrier) are, and may influence stability as well as capacity to insert the nanopore, e.g., through electroporation, pipette pump cycle, or detergent assisted nanopore insertion. Additionally, or alternatively, the ratio of molecular weights of the hydrophilic and hydrophobic blocks may affect self-assembly of those blocks into the layers of the barrier. Additionally, or alternatively, the respective glass transition temperatures (T_{g}) of the hydrophobic and hydrophilic blocks may affect the lateral fluidity of the layers of the barrier; as such, in some examples it may be useful for the hydrophobic and/or hydrophilic blocks to have a T_{g} of less than the operating temperature of the device, e.g., less than room temperature, and in some examples less than about 0° C. Additionally, or alternatively, chemical structures of the hydrophobic and hydrophilic blocks may affect the way the chains get packed into the layers, and stability of those layers.

For nanopore sequencing applications, barrier fluidity can be considered beneficial. Without wishing to be bound by any theory, the fluidity of a block copolymer barrier is believed to be largely imparted by the physical property of the hydrophobic “B” blocks. More specifically, B blocks including “low T_{g}” hydrophobic polymers (e.g., having a T_{g} below around 0° C.) may be used to generate barriers that are more fluid than those with B blocks including “high T_{g}” polymers (e.g., having a T_{g} above room temperature). For example, in certain examples, a hydrophobic B block of the copolymer has a T_{g} of less than about 20° C., less than about 0° C., or less than about −20° C.

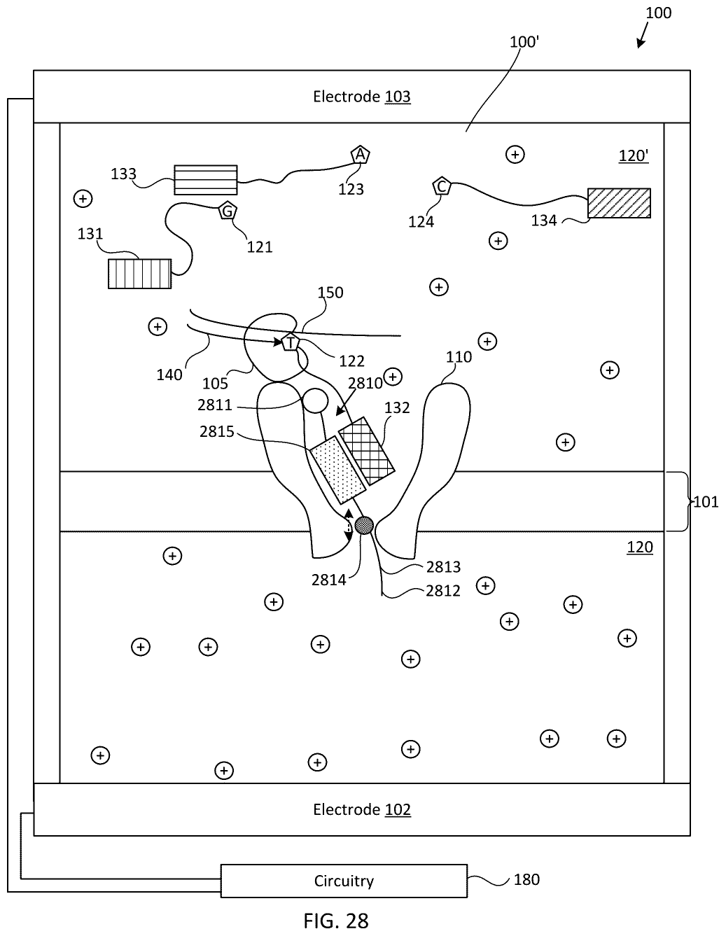

Hydrophobic B blocks with a low T_{g} may be used to help maintain barrier flexibility under conditions suitable for performing nanopore sequencing, e.g., in a manner such as described with reference to FIGS. 28-32. In some examples, hydrophobic B blocks with a sufficiently low T_{g} for use in nanopore sequencing may include, or may consist essentially of, PIB, which may be expected to have a T_{g} in the range of about −75° C. to about −25° C. In other examples, hydrophobic B blocks with a sufficiently low T_{g} for use in nanopore sequencing may include, or may consist essentially of, PDMS, which may be expected to have a T_{g} in the range of about −135° C. (or lower) to about −115° C. In still other examples, hydrophobic B blocks with a sufficiently low T_{g} for use in nanopore sequencing may include, or may consist essentially of, PBd. Different forms of PBd may be used as B blocks in the present barriers. For example, the cis-1,4 form of PBd may be expected to have a T_{g} in the range of about −105° C. to about −85° C. Or, for example, the cis-1,2 form of PBd may be expected to have a T_{g} in the range of about −25° C. to about 0° C. Or, for example, the trans-1,4 form of PBd may be expected to have a T_{g} in the range of about −95° C. to about −5° C. In yet other examples, hydrophobic B blocks with a sufficiently low T_{g} for use in nanopore sequencing may include, or may consist essentially of, polymyrcene (PMyr), which may be expected to have a T_{g} in the range of about −75° C. to about-45° C. In yet other examples, hydrophobic B blocks with a sufficiently low T_{g} for use in nanopore sequencing may include, or may consist essentially of, polyisoprene (PIP). Different forms of PIP may be used as B blocks in the present barriers. For example, the cis-1,4 form of PIP may be expected to have a T_{g} in the range of about −85° C. to about −55° C. Or, for example, the trans-1,4 form of PIP may be expected to have a T_{g} in the range of about −75° C. to about −45° C.

Hydrophobic B blocks with a fully saturated carbon backbone, such as PIB, also may be expected to increase chemical stability of the block copolymer barrier. Additionally, or alternatively, branched structures within the hydrophobic B block, such as with PIB, may be expected to induce chain entanglement, which may be expected to enhance the stability of the block copolymer barrier. This may allow for a smaller hydrophobic block to be used, ameliorating the penalty of hydrophobic mismatch towards an inserted nanopore. Additionally, or alternatively, hydrophobic B blocks with relatively low polarity may be expected to be better electrical insulators, thus improving electrical performance of a device for nanopore sequencing (e.g., such as described with reference to FIGS. 28-32).

In some examples of the AB copolymer shown below including PBd as the B block and PEO as the A block, R is reactive group 311, 1111, or 1112; m=about 2 to about 100; and n=about 2 to about 100.

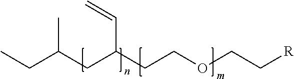

In some nonlimiting examples, R is reactive group 311, 1111, or 1112; n=about 8 to about 50; and m=about 1 to about 20. In some nonlimiting examples, R is reactive group 311, 1111, or 1112; n=about 10 to about 15; and m=about 5 to about 15.

In some examples of the ABA copolymer shown below including one or more PIB blocks as the B block and PEO as the A block, at least one of R₁ and R₂ may be reactive group 311, 1111, or 1112, and the other of R₁ and R₂ may be reactive group 311, 1111, or 1112, or may be a group which is not reactive to the chemistry which is used to react 311, 1111, or 1112; V is an optional group that corresponds to a bis-functional initiator from which the isobutylene may be propagated and can be tert-butylbenzene, a phenyl connected to the hydrophobic blocks via the para, meta, or ortho positions, naphthalene, another aromatic group, an alkane chain with between about 2 and about 20 carbons, or another aliphatic group; m=about 2 to about 100; and n=about 2 to about 100. V may in some embodiments be flanked by functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen. When V is absent, only one PIB block is present and n=about 2 to about 100. L₁ and L₂ are independently linkers, which in some examples may be nonreactive, e.g., may include at least one moiety selected from the group consisting of an amide, a thioether (sulfide), a succinic group, a maleic group, a methylene, an ether, and a product of a “click” reaction. In other examples, L₁ and/or L₂ may be reactive, and may correspond to reactive moieties 311, 1111, or 1112 and may be cross-linked in a manner similar to that described with reference to FIGS. 3A-3F. In such examples, R₁ and/or R₂ need not necessarily be reactive.

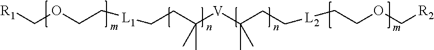

In some nonlimiting examples of the above structure, n=about 2 to about 50, and m=about 1 to about 50, V=tert-butylbenzene, and L₁=L₂=ethyl sulfide. In other nonlimiting examples, n=about 5 to about 20, m=about 2 to about 15, V=tert-butylbenzene, and L₁=L₂=ethyl sulfide. In other nonlimiting examples, n=about 13 to about 19, m=about 2 to about 5, V=tert-butylbenzene, and L₁=L₂=ethyl sulfide. In other nonlimiting examples, n=about 7 to about 13, m=about 7 to about 13, V=tert-butylbenzene, and L₁=L₂=ethyl sulfide. In particular, in one nonlimiting example, n=16, m=3, V=tert-butylbenzene, and L₁=L₂=ethyl sulfide. In another nonlimiting example, n=10, m=10, V=tert-butylbenzene, and L₁=L₂=ethyl sulfide. In another nonlimiting example, n=16, m=8, V=tert-butylbenzene, and L₁=L₂=ethyl sulfide.

In some examples, multifunctional precursors may be sourced and used as precursors to the synthesis of bifunctional initiators to which V corresponds in the example further above. For example, the multifunctional precursor may be 5-tert-butylisophthalic acid (TBIPA) which can be synthesized into 1-(tert-butyl)-3,5-bis(2-methoxypropan-2-yl)benzene (TBDMPB) using reactions known in the art. In another example, TBIPA may be synthesized into 1-tert-butyl-3,5-bis(2-chloropropan-2-yl)benzene using reactions known in the art. The use of such bifunctional initiators allows cationic polymerization on both sides of the initiator, generating bifunctional PIBs, such as allyl-PIB-allyl, which can then be coupled to hydrophilic A blocks to generate ABA block copolymers including PIB as the B block. Here, although the bifunctional initiator may be located between first and second PIB polymers, it should be understood that the first and second PIB polymers and the bifunctional initiator (V) together may be considered to form a B block, e.g., of an ABA triblock copolymer.

In another nonlimiting example, an ABA triblock copolymer includes

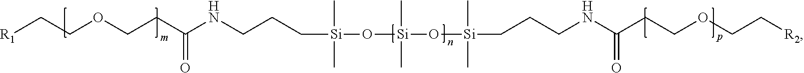

where m=about 2 to about 100, n=about 2 to about 100, p=about 2 to about 100, at least one of R₁ and R₂ may be reactive group 311, 1111, or 1112 and the other of R₁ and R₂ may be reactive group 311, 1111, or 1112 or may be a group which is not reactive to the chemistry which is used to react 311, 1111, or 1112. In some nonlimiting examples, m=about 2 to about 30, n=about 25 to about 45, and p=about 2 to about 30. In some nonlimiting examples, m=about 2 to about 15, n=about 30 to about 40, and p=about 2 to about 15. In some nonlimiting examples, m=about 7 to about 11, n=about 35 to about 40, and p=about 7 to about 11. In some nonlimiting examples, m=about 2 to about 5, n=about 30 to about 37, and p=about 2 to about 5. In particular, in one nonlimiting example, m=3, n=34, and p=3. In another nonlimiting example, m=9, n=37, and p=9.

In some examples of the AB copolymer shown below including a PIB block as the B block and PEO as the A block, R is reactive group 311, 1111, or 1112; m=about 2 to about 100; n=about 2 to about 100; and L is a linker. In some examples, L is non-reactive, e.g., is selected from the group consisting of an amide, a thioether (sulfide), a succinic group, a maleic group, a methylene, an ether, or a product of a click reaction. In other examples, L may be reactive, and may correspond to reactive moieties 311, 1111, or 1112 and may be cross-linked in a manner similar to that described with reference to FIGS. 3A-3F. In such examples, R need not necessarily be reactive.

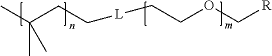

In one nonlimiting example, n=13, m=8, and L is ethyl sulfide. In another nonlimiting example, n=13, m=3, and L is ethyl sulfide. In another nonlimiting example, n=30, m=8, and L is ethyl sulfide. In another nonlimiting example, n=30, m=3, and L is ethyl sulfide.

Accordingly, it will be appreciated that a wide variety of amphiphilic molecules and a wide variety of reactive moieties may be used to generate barriers that are stabilized using covalent bonds to molecules, e.g., for use in a nanopore device such as described with reference to FIG. 1. FIG. 19 illustrates an example flow of operations in a method 1900 for forming a barrier. Method 1900 may include forming at least one layer including a plurality of amphiphilic molecules, wherein the amphiphilic molecules include reactive moieties (operation 9610). For example, operation 1910 may include forming first and second layers respectively including first and second pluralities of the amphiphilic molecules. In other examples, operation 1910 may include forming a single layer, or a layer which is partially a single layer and partially a bilayer. That is, barrier 101 may include molecules of block copolymers (e.g., AB, ABA, or BAB), which have any suitable arrangement within the barrier, such as described elsewhere herein. The hydrophilic “A” blocks, the hydrophobic “B” blocks, or the A-B interfaces of the amphiphilic molecules (e.g., block copolymers) may be coupled to reactive moieties (e.g., 311, 1111, or 1112) in a manner such as described with reference to FIGS. 3A-13B.

Method 1900 illustrated in FIG. 19 also may include using first crosslinking reactions of the reactive moieties to only crosslink amphiphilic molecules of the plurality to one another (operation 1920). In examples including first and second layers, the crosslinking reactions may be used to partially couple amphiphilic molecules of the first layer to one another and/or to amphiphilic molecules of the second layer, and/or may be used to partially crosslink amphiphilic molecules of the second layer to one another and/or to amphiphilic molecules of the first layer. In some examples of operation 1920, reactive moieties 311 may be used to only partially polymerize the amphiphilic molecules prior to nanopore insertion in a manner such as described with reference to FIGS. 3A-10B. In other examples of operation 1620, reactive moieties 1111 and 1112 may be used to partially couple the amphiphilic molecules to one another prior to nanopore insertion in a manner such as described with reference to FIGS. 11A-13B. In some embodiments, the at least one layer formed in operation 1910 further may include monomers (e.g., monomers 321 and/or 321′) which are partially polymerized in operation 1920, or in a separate operation.

Method 1900 illustrated in FIG. 19 also may include, after the first crosslinking reactions, inserting a nanopore into the at least one layer (operation 1930). Example operations for inserting a nanopore into a barrier are provided elsewhere herein. In some embodiments, the nanopore may include one or more moieties 1611 such as described with reference to FIG. 16, and/or may include one or more initiators 1511 such as described with reference to FIG. 15. Method 1900 illustrated in FIG. 19 further may include, after inserting the nanopore, using second cross-linking reactions of the reactive moieties to further crosslink amphiphilic molecules of the plurality to one another (operation 1940). In examples including first and second layers, the second crosslinking reactions may be used to couple additional amphiphilic molecules of the first layer to one another and/or to amphiphilic molecules of the second layer, and/or may be used to additionally crosslink amphiphilic molecules of the second layer to one another and/or to amphiphilic molecules of the first layer. In some embodiments, the at least one layer formed in operation 1910 further may include monomers (e.g., monomers 321 and/or 321′) which are additionally polymerized in operation 1940, or in a separate operation.

It will further be appreciated that the present barriers may be used in any suitable device or application. For example, FIG. 28 schematically illustrates a cross-sectional view of an example use of the composition and device of FIG. 1. Device 100 illustrated in FIG. 28 may be configured to include fluidic well 100′, barrier 101 which may have a configuration such as described elsewhere herein, first and second fluids 120, 120′, and nanopore 110 in a manner such as described with reference to FIG. 1. In the nonlimiting example illustrated in FIG. 28, second fluid 120′ in some embodiments may include a plurality of each of nucleotides 121, 122, 123, 124, e.g., G, T, A, and C, respectively. Each of the nucleotides 121, 122, 123, 124 in second fluid 120′ in some embodiments may be coupled to a respective label 131, 132, 133, 134 coupled to the nucleotide via an elongated body (elongated body not specifically labeled). In some embodiments, device 100 further may include polymerase 105. As illustrated in FIG. 28, polymerase 105 may be within the second composition of second fluid 120′. Alternatively, polymerase 105 may be coupled to nanopore 110 or to barrier 101, e.g., via a suitable elongated body (not specifically illustrated). Device 100 in some embodiments further may include first and second polynucleotides 140, 150 in a manner such as illustrated in FIG. 28. Polymerase 105 may be for sequentially adding nucleotides of the plurality to the first polynucleotide 140 using a sequence of the second polynucleotide 150. For example, at the particular time illustrated in FIG. 28, polymerase 105 incorporates nucleotide 122 (T) into first polynucleotide 140, which is hybridized to second polynucleotide 150 to form a duplex. At other times (not specifically illustrated), polymerase 105 sequentially may incorporate other of nucleotides 121, 122, 123, 124 into first polynucleotide 140 using the sequence of second polynucleotide 150.

Circuitry 180 illustrated in FIG. 28 may be configured to detect changes in an electrical characteristic of the aperture responsive to the polymerase sequentially adding nucleotides of the plurality to the first polynucleotide 140 using a sequence of the second polynucleotide 150. In the nonlimiting example illustrated in FIG. 28, nanopore 110 may be coupled to permanent tether 2810 which may include head region 2811, tail region 2812, elongated body 2813, reporter region 2814 (e.g., an abasic nucleotide), and moiety 2815. Head region 2811 of tether 2810 is coupled to nanopore 110 via any suitable chemical bond, protein-protein interaction, or any other suitable attachment that is normally irreversible. Head region 2811 can be attached to any suitable portion of nanopore 110 that places reporter region 2814 within aperture 2813 and places moiety 2815 sufficiently close to polymerase 105 so as to interact with respective labels 131, 132, 133, 134 of nucleotides 121, 122, 123, 124 that are acted upon by polymerase 105. Moiety 2815 respectively may interact with labels 131, 132, 133, 134 in such a manner as to move reporter region 2814 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to nanopores to sequence polynucleotides, see U.S. Pat. No. 9,708,655, the entire contents of which are incorporated by reference herein.

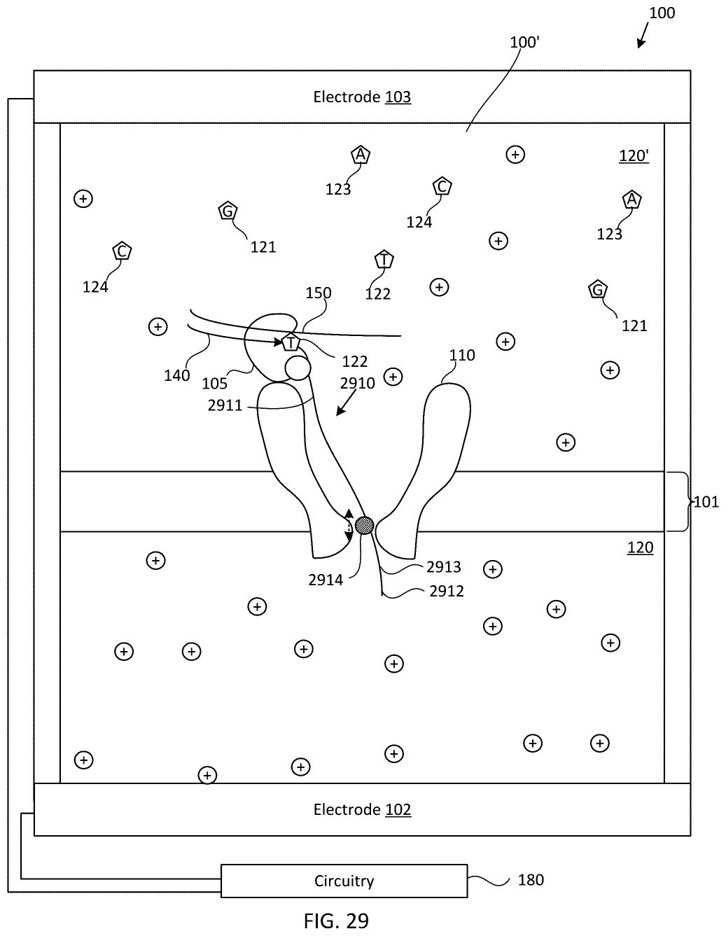

FIG. 29 schematically illustrates a cross-sectional view of another example use of the composition and device of FIG. 1. As illustrated in FIG. 29, device 100 may include fluidic well 100′, barrier 101 which may have a configuration such as described elsewhere herein, first and second fluids 120, 120′, nanopore 110, and first and second polynucleotides 140, 150, all of which may be configured similarly as described with reference to FIG. 28. In the nonlimiting example illustrated in FIG. 29, nucleotides 121, 122, 123, 124 need not necessarily be coupled to respective labels. Polymerase 105 may be coupled to nanopore 110 and may be coupled to permanent tether 2910 which may include head region 2911, tail region 2912, elongated body 2913, and reporter region 2914 (e.g., an abasic nucleotide). Head region 2911 of tether 2910 is coupled to polymerase 105 via any suitable chemical bond, protein-protein interaction, or any other suitable attachment that is normally irreversible. Head region 2911 can be attached to any suitable portion of polymerase 105 that places reporter region 2914 within aperture 113. As polymerase 105 interacts with nucleotides 121, 122, 123, 124, such interactions may cause polymerase 105 to undergo conformational changes. Such conformational changes may move reporter region 2914 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to polymerases to sequence polynucleotides, see U.S. Pat. No. 9,708,655, the entire contents of which are incorporated by reference herein.

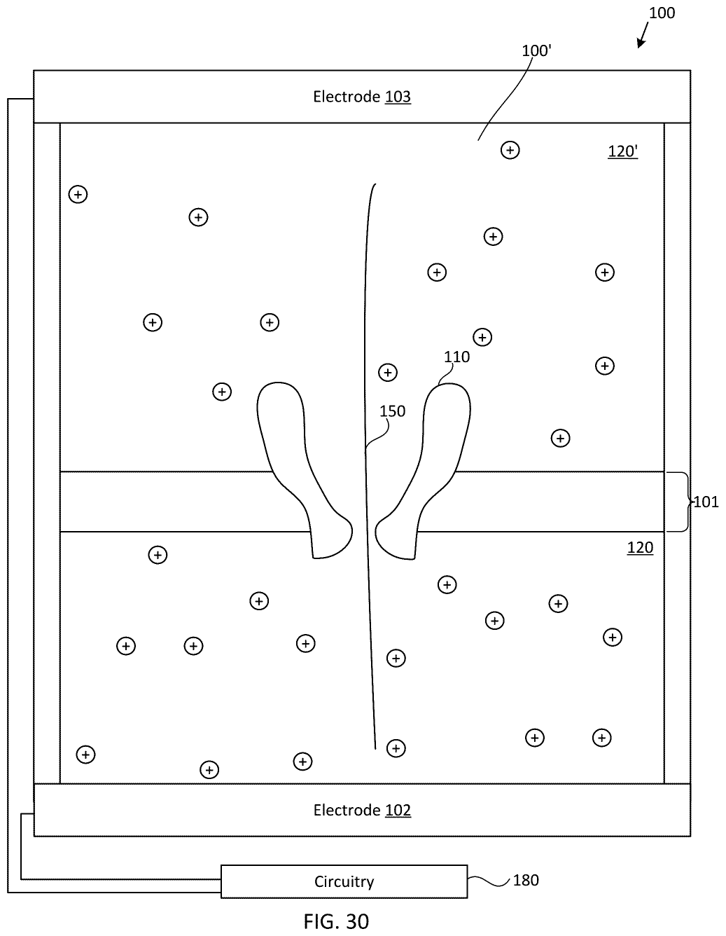

FIG. 30 schematically illustrates a cross-sectional view of another example use of the composition and device of FIG. 1. As illustrated in FIG. 30, device 100 may include fluidic well 100′, barrier 101 which may have a configuration such as described elsewhere herein, first and second fluids 120, 120′, and nanopore 110 all of which may be configured similarly as described with reference to FIG. 28. In the nonlimiting example illustrated in FIG. 30, polynucleotide 150 is translocated through nanopore 110 under an applied force, e.g., a bias voltage that circuitry 180 applies between electrode 102 and electrode 103. As bases in polynucleotide 150 pass through nanopore 110, such bases may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see U.S. Pat. No. 5,795,782, the entire contents of which are incorporated by reference herein. In some examples, a helicase is used to translocate polynucleotide 150 through nanopore 110 in a stepwise manner, so as to facilitate distinguishing the bases in polynucleotide 150 from one another. In other examples, an exonuclease is used to sequentially cleave nucleotides from polynucleotide 150, and as the bases pass through aperture 113 they are detected.

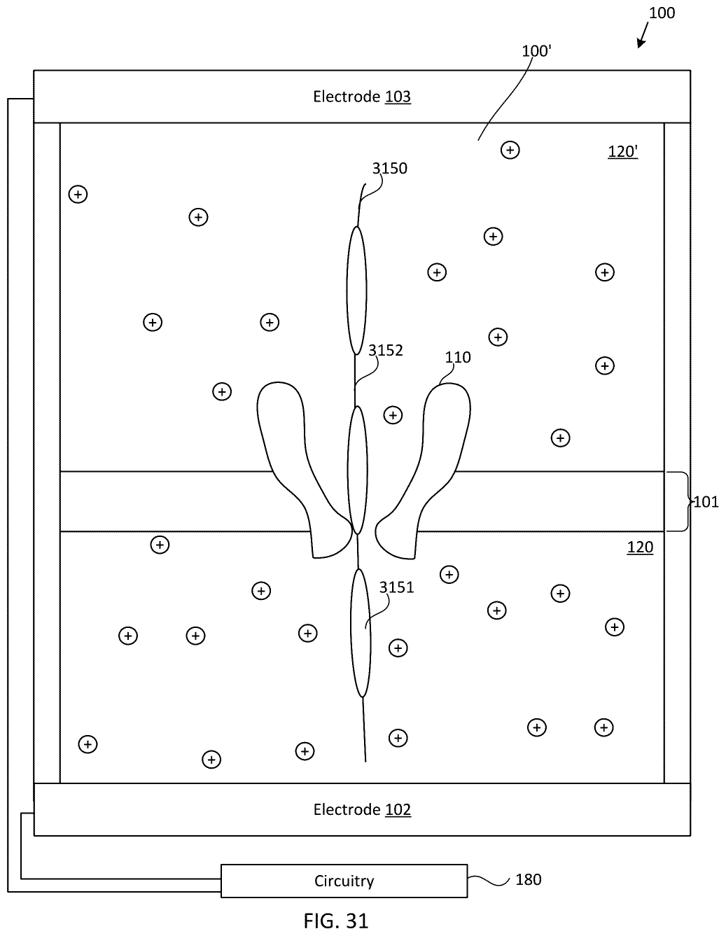

FIG. 31 schematically illustrates a cross-sectional view of another example use of the composition and device of FIG. 1. As illustrated in FIG. 31, device 100 may include fluidic well 100′, barrier 101 which may have a configuration such as described elsewhere herein, first and second fluids 120, 120′, and nanopore 110 all of which may be configured similarly as described with reference to FIG. 28. In the nonlimiting example illustrated in FIG. 31, surrogate polymer 3150 is translocated through nanopore 110 under an applied force, e.g., a bias voltage that circuitry 180 applies between electrode 102 and electrode 103. As used herein, a “surrogate polymer” is intended to mean an elongated chain of labels having a sequence corresponding to a sequence of nucleotides in a polynucleotide. In the example illustrated in FIG. 31, surrogate polymer 3150 includes labels 3151 coupled to one another via linkers 3152. An XPANDOMER™ is a particular type of surrogate polymer developed by Roche Sequencing, Inc. (Pleasanton, CA). XPANDOMERS™ may be prepared using Sequencing By eXpansion™ (SBX™, Roche Sequencing, Pleasanton CA). In Sequencing by eXpansion™, an engineered polymerase polymerizes xNTPs which include nucleobases coupled to labels via linkers, using the sequence of a target polynucleotide. The polymerized nucleotides are then processed to generate an elongated chain of the labels, separated from one another by linkers which are coupled between the labels, and having a sequence that is complementary to that of the target polynucleotide. For example descriptions of XPANDOMERS™, linkers (tethers), labels, engineered polymerases, and methods for SBX™, see the following patents, the entire contents of each of which are incorporated by reference herein: U.S. Pat. Nos. 7,939,249, 8,324,360, 8,349,565, 8,586,301, 8,592,182, 9,670,526, 9,771,614, 9,920,386, 10,301,345, 10,457,979, 10,676,782, 10,745,685, 10,774,105, and 10,851,405.

FIG. 32 schematically illustrates a cross-sectional view of another example use of the composition and device of FIG. 1. As illustrated in FIG. 32, device 100 may include fluidic well 100′, barrier 101 which may have a configuration such as described elsewhere herein, first and second fluids 120, 120′, and nanopore 110 all of which may be configured similarly as described with reference to FIG. 1. In the nonlimiting example illustrated in FIG. 32, a duplex between polynucleotide 140 and polynucleotide 150 is located within nanopore 110 under an applied force, e.g., a bias voltage that circuitry 180 applies between electrode 102 and electrode 103. A combination of bases in the double-stranded portion (here, the base pair GC 121, 124 at the terminal end of the duplex) and bases in the single-stranded portion of polynucleotide 150 (here, bases A and T 123, 122) may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see US Patent Publication No. 2023/0090867 to Mandell et al., the entire contents of which are incorporated by reference herein.

### WORKING EXAMPLES

The following example is intended to be purely illustrative, and not limiting of the present invention.

The following PEO-b-PDMS-b-PEO ABA block copolymer (referred to as PEO-PDMS-PEO for short) was prepared that included a polymerizable maleic group at each of the A-B interfaces, and that included hydroxyl end groups:

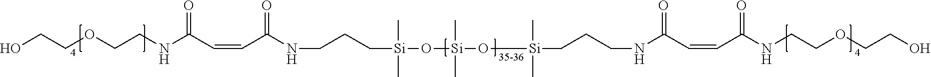

In some examples, the block copolymer was dissolved in an organic solvent consisting essentially of acrylate monomers, specifically laurel methacrylate and 1,4-butanediol dimethacrylate (3:1 v:v). In other examples, the polymer was dissolved in an organic solvent consisting essentially of 95:5 octane:butanol. The mixture of the organic solvent and PEO-PDMS-PEO, and an aqueous buffer that included 0.3 w % Irgacure 2959 as an initiator, were used to form barriers using painting in a manner such as described with reference to FIGS. 3A-3B.

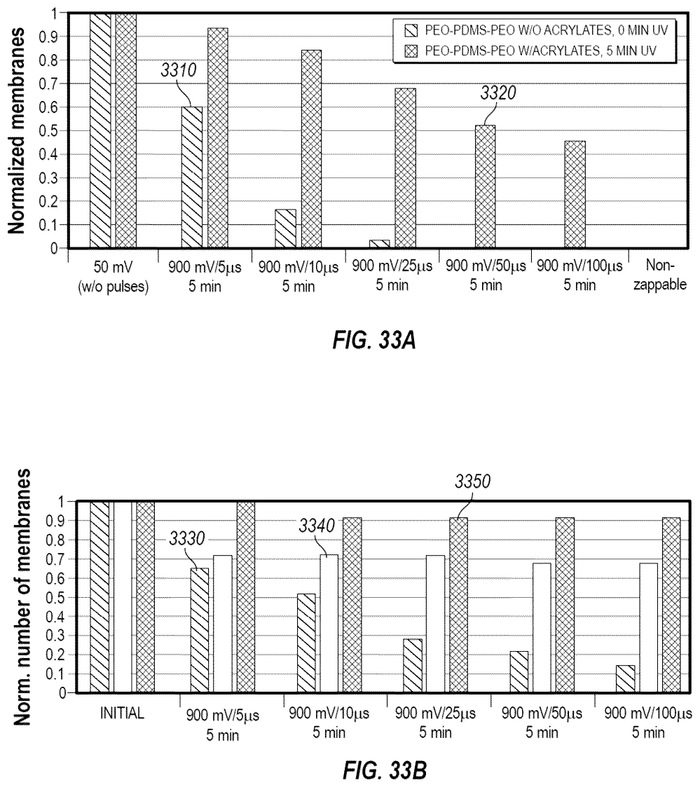

The barriers were partially or fully crosslinked using polymerization under a variety of conditions in a manner such as described with reference to FIG. 3C. FIGS. 33A-33B are plots illustrating the measured stability of barriers formed in the manner described with reference to FIGS. 3A-3C. More specifically, the normalized number of surviving barriers was measured when subjected to a waveform made of a train of positive voltage micro pulses, spaced by ejecting periods at −100 mV for 100 ms. The train of +900 mV voltage pulses has a total of 20 pulses in this set up, with duration of 5 μs. The spacings between them (reading steps) have a set duration value of 30 ms and a voltage held at +50 mV. During a first cycle, the protocol was applied continuously for a period of 5 minutes and the magnitude of the pulses was kept at +900 mV. Further applied cycles (applied again for 5 mins each), are characterized by the increasing of the pulsing duration from 5 us to 10 μs, 25 μs, 50 us and 100 μs, for a total of five cycles.

In FIG. 33A, plot 3310 illustrates the normalized number of surviving barriers (membranes) for a first set of barriers that was prepared without acrylates and without exposure to UV light, and plot 3320 illustrates the normalized number of surviving barriers for a second set of barriers that was prepared using the acrylate solvent and using a 5 minute exposure to UV light. From plot 3310 in FIG. 33A, it may be seen that the barriers which were made without the acrylate solvent and without UV light were stable under the 50 mV voltage, and that the normalized number of surviving barriers decreased to about 0.6 at the 5 μs pulse length, to about 0.17 at the 10 μs pulse length, to about 0.03 at the 25 μs pulse length, and to zero for greater pulse lengths. From plot 3320 in FIG. 33A, it may be seen that the barriers which were made with the acrylate solvent and with 5 minutes of UV light exposure were stable under the 50 mV voltage, and that the normalized number of surviving barriers decreased to about 0.95 at the 5 us pulse length, to about 0.85 at the 10 μs pulse length, to about 0.69 at the 25 μs pulse length, to about 0.53 at the 50 μs pulse length, and to about 0.45 at the 100 μs pulse length. Comparing plot 3310 to plot 3320, it may be understood that the barriers made with the acrylate solvent and with 5 minutes of UV light exposure were significantly more stable under all pulsed segments of the waveform than the barriers made without acrylate solvent and without UV light exposure.

In FIG. 33B, plot 3330 illustrates the normalized number of surviving barriers (membranes) for a first set of barriers that was prepared with acrylates without exposure to UV light, plot 3340 illustrates the normalized number of surviving barriers for a second set of barriers that was prepared using the acrylate solvent and using a 10 minute exposure to UV light, and plot 3350 illustrates the normalized number of surviving barriers for a second set of barriers that was prepared using the acrylate solvent and using a 10 minute exposure to UV light. From plot 3330 in FIG. 33B, it may be seen that the barriers which were made with the acrylate solvent without exposure to UV light were stable under the 50 mV voltage, and that the normalized number of surviving barriers decreased to about 0.65 at the 5 μs pulse length, to about 0.52 at the 10 μs pulse length, to about 0.29 at the 25 μs pulse length, to about 0.22 at the 50 μs pulse length, and to about 0.17 at the 100 μs pulse length. From plot 3340 in FIG. 33B, it may be seen that the barriers which were made with the acrylate solvent and with 10 minutes of UV light exposure were stable under the 50 mV voltage, and that the normalized number of surviving barriers decreased to about 0.72 at the 5 μs pulse length, remained about 0.72 at the 10 μs pulse length, remained about 0.72 at the 25 μs pulse length, decreased to about 0.68 at the 50 μs pulse length, and remained about 0.68 at the 100 μs pulse length. From plot 3350 in FIG. 33B, it may be seen that the barriers which were made with the acrylate solvent and with 20 minutes of UV light exposure were stable under the 50 mV voltage, and that the normalized number of surviving barriers remained about 1.0 at the 5 μs pulse length, decreased to about 0.92 at the 10 μs pulse length, remained about 0.92 at the 25 μs pulse length, remained about 0.92 at the 50 μs pulse length, and remained about 0.92 at the 100 μs pulse length. Comparing plot 3350 to plots 3330 and 3340, it may be understood that the barriers made with the acrylate solvent and with 20 minutes of UV light exposure were significantly more stable under all pulsed segments of the waveform than the barriers made with shorter UV light exposure times.

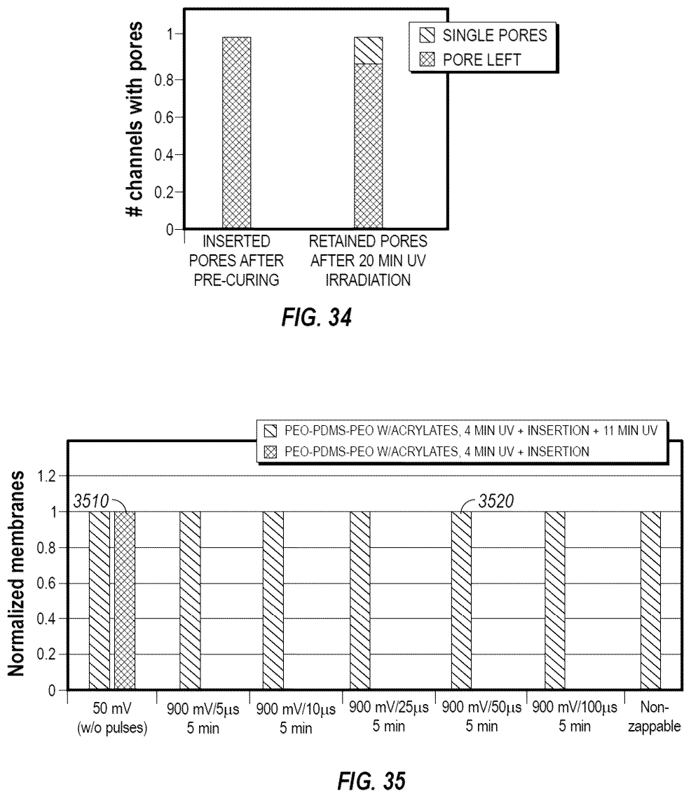

Nanopores were inserted into selected barriers. FIG. 34 is a plot illustrating the normalized number of nanopores remaining in the barrier during operations described with reference to FIGS. 3A-3F. More specifically, barriers were prepared using the acrylate solvent and exposed to UV light for 5 minutes to partially polymerize the barrier, then an MspA nanopore was inserted into the barrier. Then, the buffer solution was refreshed and the barrier exposed to UV light for another 20 minutes. From FIG. 34, it can be seen that the normalized number of channels with nanopores was 1.0 after inserting the nanopores, and was about 0.9 after exposing the barrier to UV light for another 20 minutes. Accordingly, from FIG. 34 it may be understood that a large number of nanopores remained within the barrier both before and after the second UV exposure operation.

FIG. 35 is a plot illustrating normalized number of nanopores remaining in a barrier formed in the manner described with reference to FIGS. 3A-3F under different applied voltages, namely under the waveform described with reference to FIGS. 33A-33B. In FIG. 35, plot 3520 illustrates the normalized number of nanopores for a first set of barriers that was prepared with 4 minutes of UV exposure, followed by nanopore insertion, followed by another 11 minutes of UV exposure; and plot 3510 illustrates the normalized number of surviving nanopores for a second set of barriers that was prepared with 4 minutes of UV exposure, followed by nanopore insertion, and without the second dose of UV exposure. From plot 3510 in FIG. 35, it may be seen that the barriers which were made without the second dose of UV light after nanopore insertion were stable under the 50 mV voltage, and that the normalized number of surviving nanopores decreased to about zero at all pulse lengths. From plot 3520 in FIG. 35, it may be seen that the barriers which were made with the 11 minutes of UV light exposure after nanopore insertion were completely stable (normalized number of MspA=1.0) at all voltages and pulse lengths. Comparing plot 3510 to plot 3520, it may be understood that the barriers made using UV light exposure both before and after nanopore insertion were significantly more stable under all pulsed segments of the waveform than the barriers made without a second dose of UV light exposure after nanopore insertion.

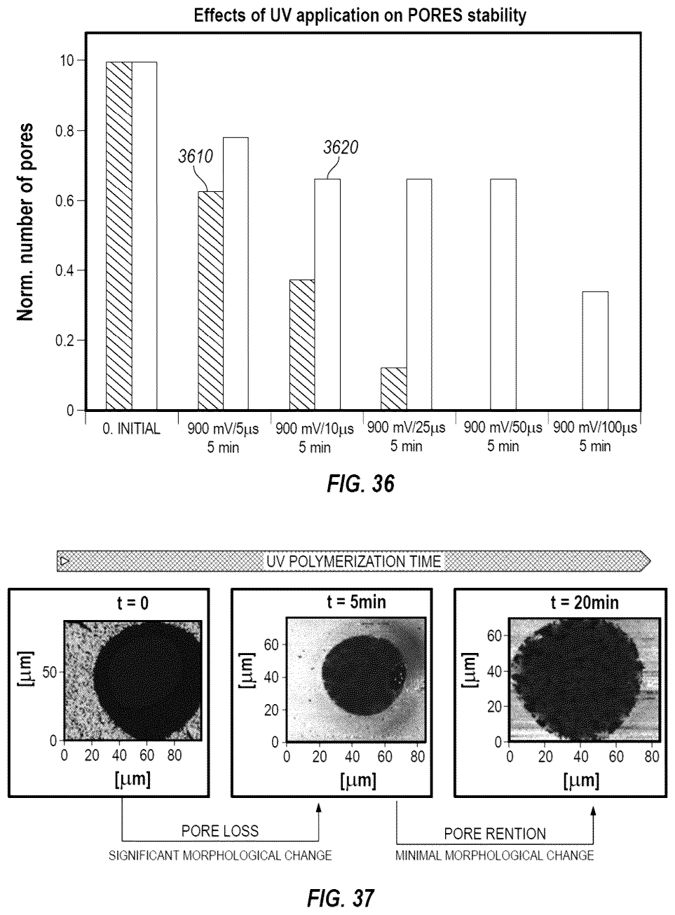

FIG. 36 is a plot illustrating normalized number of nanopores remaining in a barrier formed in the manner described with reference to FIGS. 3A-3F using different processing parameters, under different applied voltages, namely under the waveform described with reference to FIGS. 33A-33B. In FIG. 36, plot 3610 illustrates the normalized number of nanopores for a first set of barriers that was prepared with a 2 minute UV exposure, followed by nanopore insertion, without a second dose of UV exposure; and plot 3620 illustrates the normalized number of surviving barriers for a second set of barriers that was prepared with 2 minutes of UV exposure, followed by nanopore insertion, and then with a 15 minute dose of UV exposure. From plot 3610 in FIG. 36, it may be seen that the barriers which were made without the second dose of UV light after nanopore insertion were stable under the 50 mV voltage, and that the normalized number of surviving nanopores decreased to about 0.65 at the 5 μs pulse length, to about 0.4 at the 10 μs pulse length, to about 0.1 at the 25 μs pulse length, and to about zero at greater pulse lengths. From plot 3620 in FIG. 36, it may be seen that the barriers which were made with the 15 minutes of UV light exposure after nanopore insertion were stable under the 50 mV voltage, and that the normalized number of surviving barriers decreased to about 0.8 at the 5 μs pulse length, decreased to about 0.7 at the 10 μs pulse length, remained about 0.7 at the 25 μs pulse length, remained about 0.7 at the 50 μs pulse length, and decreased to about 0.35 at the 100 μs pulse length. Comparing plot 3610 to plot 3620, it may be understood that the barriers made using UV light exposure both before and after nanopore insertion were significantly more stable under all pulsed segments of the waveform than the barriers made without a second dose of UV light exposure after nanopore insertion.

FIG. 37 illustrates a stiffness profile obtained using atomic force microscopy (AFM) imaging of suspended barriers after different operations described with reference to FIGS. 3A-3F. FIG. 37 shows evidence of polymerization induced morphological changes that were obtained by imaging the suspended barriers using AFM prior to polymerization (t=0), after a five-minute UV exposure (t=5), and after a 20-minute second UV exposure. The images correlate the change in the barrier stiffness profiles with the UV exposure time. It was observed that a significant morphological change took place between t=0 and t=5, where a general stiffening of the annulus region was observed, and some stiffening of the bilayer taking place. On the other hand, in this example, minimal change was observed between t=5 and t=20, where mainly a further stiffening of the bilayer region was observed. This can be indirectly correlated with the observed nanopore behavior when inserted at a specific time on the UV exposure scale. Without wishing to be bound by any theory, the nanopore loss observed when insertion is done prior to the first UV exposure (operation 1920 in FIG. 19) can be attributed to the significant morphological changes that take place between, for example, t=0 and t=5. Doing the nanopore insertion after these morphological changes have been performed, but before the barrier becomes too stiff, is believed to facilitate the nanopore being retained with a higher success rate during the subsequent UV exposure (operation 1940 in FIG. 19), where fewer morphological changes are performed within the barrier.

Accordingly, based on the foregoing, it is believed that the present barriers will have sufficient durability for use in commercial-scale nanopore sequencing.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of forming a barrier between first and second fluids, the method comprising:
forming at least one layer comprising a plurality of amphiphilic molecules, wherein the amphiphilic molecules comprise reactive moieties;
using first crosslinking reactions of the reactive moieties to only partially crosslink amphiphilic molecules of the plurality to one another;
after using the first crosslinking reactions, inserting the nanopore into the at least one layer; and
after inserting the nanopore, using second crosslinking reactions of the reactive moieties to further crosslink amphiphilic molecules of the plurality to one another.

2. The method of claim 1, wherein forming the at least one layer comprises forming a first layer comprising a first plurality of the amphiphilic molecules, and forming a second layer comprising a second plurality of the amphiphilic molecules.

3. The method of claim 1, wherein the crosslinking reaction comprises a polymerization reaction or a coupling reaction.

4-17. (canceled)

18. The method of claim 1, wherein the reactive moieties are located at hydrophilic blocks of the amphiphilic molecules.

19. The method of claim 1, wherein the reactive moieties are located at interfaces between hydrophilic blocks and hydrophobic blocks of the amphiphilic molecules.

20. The method of claim 1, wherein the reactive moieties are located at hydrophilic blocks of the amphiphilic molecules.

21. The method of claim 1, wherein the amphiphilic molecules have an AB architecture.

22. The method of claim 1, wherein the amphiphilic molecules have an ABA architecture.

23. The method of claim 1, wherein the amphiphilic molecules have a BAB architecture.

24. The method of claim 1, wherein the amphiphilic molecules comprise poly(dimethyl siloxane) (PDMS) or poly(isobutylene) (PIB).

25. (canceled)

26. The method of claim 1, wherein the amphiphilic molecules comprise poly(ethylene oxide) (PEO).

27. The method of claim 1, wherein:
the at least one layer is formed using a hydrophobic liquid consisting essentially of hydrophobic, polymerizable monomers; and
the hydrophobic liquid is disposed within the at least one layer.

28. The method of claim 27, wherein the first cross-linking reactions at least partially crosslink the monomers with one another, or at least partially crosslink the monomers with amphiphilic molecules of the plurality.

29. (canceled)

30. The method of claim 27, wherein the second cross-linking reactions at least partially crosslink the monomers with one another, or at least partially crosslink the monomers with amphiphilic molecules of the plurality.

31. (canceled)

32. The method of claim 27, wherein at least some of the monomers include a single reactive moiety via which those monomers polymerize with other monomers or react with the reactive moieties of the amphiphilic molecules, or wherein at least some of the monomers include two or more reactive moieties via which those monomers polymerize with other monomers or react with the reactive moieties of the amphiphilic molecules.

33. (canceled)

34. The method of claim 27, wherein at least a portion of the monomers is intercalated between amphiphilic molecules of the at least one layer, or wherein the at least one layer comprises first and second layers, and wherein at least a portion of the monomers is disposed between the first layer and the second layer.

35. (canceled)

36. The method of claim 27, wherein the monomers comprise acrylate, and wherein the polymer comprises polyacrylate.

37-40. (canceled)

41. The method of claim 27, wherein the nanopore comprises a moiety that initiates the polymerization, or wherein the nanopore comprises a moiety that couples to a reactive moiety of an amphiphilic molecule.

42. (canceled)

43. The method of claim 1, wherein the nanopore comprises α-hemolysin or MspA.

44. (canceled)

45. A barrier between first and second fluids, the barrier comprising:
at least one layer comprising a plurality of amphiphilic molecules and a polymer,
wherein at least some amphiphilic molecules of the plurality of amphiphilic molecules are crosslinked to one another, and wherein at least some amphiphilic molecules of the plurality of amphiphilic molecules are crosslinked to the polymer.

46-68. (canceled)

69. A barrier between first and second fluids, the barrier comprising:
at least one layer comprising a plurality of amphiphilic molecules,
wherein the amphiphilic molecules comprise reactive moieties to perform a crosslinking reaction with one another, and
wherein only a subset of the amphiphilic molecules are crosslinked with one another via a reaction product of the crosslinking reaction; and
a nanopore within the barrier.

70-97. (canceled)
